(19) United States
(12) Patent Application Publication
(10) Pub. No.: US 2017/0233485 A1
(43) Pub. Date: Aug. 17, 2017

(54) ANTI-CD40 ANTIBODIES AND USES THEREOF

(71) Applicant: Biogen MA Inc., Cambridge, MA (US)

(72) Inventors: Linda C. Burkly, Monument Beach, MA (US); Janine Ferrant-Orgettas, Gloucester, MA (US)

(21) Appl. No.: 15/504,444

(22) PCT Filed: Aug. 18, 2015
(86) PCT No.: PCT/US2015/045748

§ 371 Date: Feb. 16, 2017

Related U.S. Application Data
(60) Provisional application No. 62/038,773, filed on Aug. 18, 2014.

(51) Int. Cl. C07K 16/28

(52) U.S. Cl. CPC C07K 16/2878; C07K 2317/92; C07K 2317/76; C07K 2317/565; C07K 2317/75; C07K 2317/24; A61K 2039/505

(57) ABSTRACT

Antibodies and antibody fragments that bind to human CD40 and inhibit interaction between CD40 and its ligand, CD40L are disclosed. Also disclosed are methods of using the antibodies and antibody fragments to inhibit hyperactivation of B or T cells and treat or prevent disorders such as autoimmune diseases.

### CROSS-REFERENCE TO RELATED APPLICATIONS

This application claims the benefit of priority of U.S. Provisional Patent Appl. No. 62/038,773, filed Aug. 18, 2014, the contents of which are incorporated by reference herein in their entirety.

### BACKGROUND

CD40 is a Type 1 transmembrane receptor expressed by B cells, macrophages, dendritic cells, and other cell types, including platelets, epithelial, endothelial, and stromal cells. The engagement of CD40 by its ligand, CD40 ligand (CD40L also known as CD154), constitutes a key axis for the activation of innate and adaptive immune functions. This notably includes B cell functions of clonal expansion, differentiation to antibody forming cells (AFC) and memory cells expressing isotype-switched antibodies, and the germinal center (GC) reaction. Thus, CD40/CD40L is a premier immunological pathway that affects processes thought to be involved in diseases of autoimmunity and humoral immunity (Burkly, Adv. Exp. Med. Biol., 489:135-52 (2001); van Kooten et al., J. Leuk. Biol., 67:2-17 (2000)). Therefore, antibodies that modulate the CD40/CD40L interaction are of interest in treating diseases such as autoimmune and inflammatory diseases.

### SUMMARY

This disclosure relates to anti-CD40 antibodies and their uses. These antibodies bind to human CD40 and inhibit interaction between CD40 and its ligand, CD40L. These antibodies are useful to inhibit hyperactivation of B or T cells and treat or prevent disorders such as autoimmune and inflammatory diseases.

In one aspect, this disclosure provides an isolated antibody or antigen-binding fragment thereof that selectively binds to human CD40 and both (i) binds to the same epitope on human CD40 as an antibody that has a heavy chain comprising amino acids 21-463 of SEQ ID NO:46 and a light chain comprising amino acids 23-236 of SEQ ID NO:38, and (ii) inhibits the interaction between human CD40 and human CD40 ligand. In certain embodiments, the antibody or antigen-binding fragment thereof inhibits the humoral response to tetanus toxoid immunization in a primate without B cell depletion compared to vehicle and/or does not elevate IL-12 serum levels in a primate compared to vehicle and/or binds to a CD40 protein encoded by a DNA molecule containing the CD40 SNP H78Q comparably as to wild type human CD40 (SEQ ID NO:58).

In another aspect, this disclosure provides an isolated antibody or antigen-binding fragment thereof that selectively binds to human CD40. This antibody or antigen-binding fragment thereof cross-blocks an antibody that has a heavy chain comprising amino acids 21-463 of SEQ ID NO:46 and a light chain comprising amino acids 23-236 of SEQ ID NO:38; inhibits the interaction between human CD40 and human CD40 ligand; and inhibits the humoral response to tetanus toxoid immunization in a primate without B cell depletion compared to vehicle. This antibody or antigen-binding fragment thereof also does not elevate IL-12 serum levels in a primate compared to vehicle and/or binds to a CD40 protein encoded by a DNA molecule containing the CD40 SNP H78Q comparably as to wild type human CD40 (SEQ ID NO:58).

In a further aspect, this application discloses an isolated antibody or antigen-binding fragment thereof that selectively binds to a conformational epitope within cysteine-rich domain 2 (CRD2) and cysteine-rich domain 3 (CRD3) of human CD40. This antibody or antigen-binding fragment thereof inhibits the humoral response to tetanus toxoid immunization in a primate without B cell depletion compared to vehicle; and/or does not elevate IL-12 serum levels in a primate compared to vehicle; and/or binds to a CD40 protein encoded by a DNA molecule containing the CD40 SNP C77F about 50% as well as to wild type human CD40 (SEQ ID NO:58); and/or binds to a CD40 protein encoded by a DNA molecule containing the CD40 SNP H78Q comparably as to wild type human CD40 (SEQ ID NO:58). This antibody or antigen-binding fragment thereof optionally has one or more of the following functions/activities: (i) inhibits the interaction between human CD40 and human CD40 ligand; (ii) has a KD≦3 nM for cysteine-rich domains 2-3 of the extracellular domain of human CD40; (iii) has an EC50 value between 20 and 200 ng/mL for binding to B cells in human whole blood; (iv) inhibits primary B cell activation by CD40L on Jurkat cells with an IC50 of between 5 and 100 ng/mL; (v) inhibits primary B cell activation in whole blood by soluble CD40L with an IC50 of between 10 and 200 ng/mL; (vi) does not agonize platelets stimulated by soluble CD40L compared with the anti-CD40 antibody, G28.5 antibody; (vii) has less agonistic activity in a RAMOS B cell line compared to the anti-CD40 antibody, ADH9; (viii) has less agonistic activity in whole blood cultures compared to the anti-CD40 antibody, ADH9; (ix) has reduced binding as compared to a wild type IgG1 to CD16a of about 200 fold, to CD32a and CD32b of about 5 fold, and CD64 of about 150 fold; and/or (x) binds to a CD40 protein encoded by a DNA sequence that contains at least one of the following human CD40 SNPs: A25S; S124L; I134V; F158L; and S166R comparably as to wild type human CD40 (SEQ ID NO:58).

In some embodiments of the above aspects, the isolated antibody or antigen-binding fragment thereof comprises a heavy chain CDR1 comprising/consisting of the amino acid sequence TFPIE (SEQ ID NO: 61); a heavy chain CDR2 comprising/consisting of the amino acid sequence NFHPYNDDTKYNEKFKG (SEQ ID NO:62); and a heavy chain CDR3 comprising/consisting of the amino acid sequence RGKLPFDS (SEQ ID NO:63). In certain instances, the isolated antibody or antigen-binding fragment thereof further comprises at least two of the light chain CDRs comprising/consisting of the amino acid sequences set forth in SEQ ID NOs.: 64, 65, and 66. In certain embodiments, the anti-human CD40 antibody or antigen-binding fragment thereof comprises a VH domain that is at least 90%, 91%, 92%, 93%, 94%, 95%, 96%, 97%, 98%, 99%, or 100% identical to the amino acid sequence of SEQ ID NO:33. In a specific embodiment, such a VH domain comprises the heavy chain CDR1 comprising/consisting of the amino acid sequence TFPIE (SEQ ID NO: 61); the heavy chain CDR2 comprising/consisting of the amino acid sequence NFHPYNDDTKYNEKFKG (SEQ ID NO:62); the heavy chain CDR3 comprising/consisting of the amino acid sequence RGKLPFDS (SEQ ID NO:63). In another embodiment, the anti-human CD40 antibody or antigen-binding fragment thereof further comprises a VL domain that is at least 90%, 91%, 92%, 93%, 94%, 95%, 96%, 97%, 98%, 99%, or 100% identical to the amino acid sequence of SEQ ID NO:34. In a specific embodiment, such a VL domain comprises the light chain CDR1 comprising/consisting of the amino acid sequence RASQDISNYLN (SEQ ID NO:64); the light chain CDR2 comprising/consisting of the amino acid sequence FTSRLRS (SEQ ID NO:65); and the light chain CDR3 comprising/consisting of the amino acid sequence QQDRKLPWT (SEQ ID NO:66). In certain instances, the isolated antibody has reduced afucose content (e.g., 0.1% to 1.5% afucose). In some instances, the isolated antibody or antigen-binding fragment thereof have reduced galactose content and/or reduced high mannose content compared to reference anti-CD40 antibodies.

In certain embodiments of the above aspects, the antibody or antigen-binding fragment thereof also binds to cynomolgus CD40. Such an antibody or antigen-binding fragment thereof binds to rhesus CD40, murine CD40, and rat CD40 with a lower binding affinity than to human or cynomolgus CD40.

In certain embodiments of the above aspects, the antibody or antigen-binding fragment thereof binds to human CD40 at an epitope within amino acids 70 to 130 of SEQ ID NO:58; inhibits the interaction between human CD40 and human CD40 ligand; has a KD of 0.1 nM to 3 nM for CRDs 2-3 of the extracellular domain of human CD40; binds to a CD40 protein encoded by a DNA molecule containing the CD40 SNP C77F about 50% as well as to wild type human CD40 (SEQ ID NO:58); and binds to a CD40 protein encoded by a DNA molecule containing the CD40 SNP H78Q comparably as to wild type human CD40 (SEQ ID NO:58).

In another aspect, this disclosure provides an isolated antibody or antigen-binding fragment thereof that selectively binds to human CD40 and comprises a variable heavy (VH) domain comprising a heavy chain complementarity determining region 1 (CDR1), a heavy chain CDR2, and a heavy chain CDR3. In certain embodiments, the heavy chain CDR1 comprises/consists of the amino acid sequence TFPIE (SEQ ID NO: 61) or the amino acid sequence set forth in SEQ ID NO: 61 with a substitution at one or two amino acid positions; the heavy chain CDR2 comprises/consists of the amino acid sequence NFHPYNDDTKYNEKFKG (SEQ ID NO:62) or the amino acid sequence set forth in SEQ ID NO:62 with a substitution at one, two, three, or four amino acid positions; and the heavy chain CDR3 comprises/consists of the amino acid sequence RGKLPFDS (SEQ ID NO:63) or the amino acid sequence set forth in SEQ ID NO:63 with a substitution at one, two, or three amino acid positions.

In certain embodiments of the above aspect, the isolated antibody or antigen-binding fragment thereof comprises a heavy chain CDR1 comprising/consisting of the amino acid sequence TFPIE (SEQ ID NO: 61) or the amino acid sequence set forth in SEQ ID NO: 61 with a substitution at one amino acid position; a heavy chain CDR2 comprising/consisting of the amino acid sequence NFHPYNDDTKYNEKFKG (SEQ ID NO:62) or the amino acid sequence set forth in SEQ ID NO:62 with a substitution at one amino acid position; and a heavy chain CDR3 comprising/consisting of the amino acid sequence RGKLPFDS (SEQ ID NO:63) or the amino acid sequence set forth in SEQ ID NO:63 with a substitution at one amino acid position. In some embodiments, the isolated antibody or antigen-binding fragment thereof comprises a heavy chain CDR1 comprising/consisting of the amino acid sequence TFPIE (SEQ ID NO: 61); a heavy chain CDR2 comprising/consisting of the amino acid sequence NFHPYNDDTKYNEKFKG (SEQ ID NO:62); and a heavy chain CDR3 comprising/consisting of the amino acid sequence RGKLPFDS (SEQ ID NO:63). In certain embodiments, the isolated antibody or antigen-binding fragment thereof further comprises a variable light (VL) domain comprising a light chain CDR1, a light chain CDR2, and a light chain CDR3. In certain instances, the light chain CDR1 comprises/consists of the amino acid sequence RASQDISNYLN (SEQ ID NO:64) or the amino acid sequence set forth in SEQ ID NO:64 with a substitution at one, two, three, or four amino acid positions; the light chain CDR2 comprises/consists of the amino acid sequence FTSRLRS (SEQ ID NO:65) or the amino acid sequence set forth in SEQ ID NO:65 with a substitution at one or two amino acid positions; and the light chain CDR3 comprises/consists of the amino acid sequence QQDRKLPWT (SEQ ID NO:66) or the amino acid sequence set forth in SEQ ID NO:66 with a substitution at one, two, or three amino acid positions. In another embodiment, the isolated antibody or antigen-binding fragment thereof comprises a light chain CDR1 comprising/consisting of the amino acid sequence RASQDISNYLN (SEQ ID NO:64) or the amino acid sequence set forth in SEQ ID NO:64 with a substitution at one amino acid position; a light chain CDR2 comprising/consisting of the amino acid sequence FTSRLRS (SEQ ID NO:65) or the amino acid sequence set forth in SEQ ID NO:65 with a substitution at one amino acid position; and a light chain CDR3 comprising/consisting of the amino acid sequence QQDRKLPWT (SEQ ID NO:66) or the amino acid sequence set forth in SEQ ID NO:66 with a substitution at one amino acid position. In one embodiment, the isolated antibody or antigen-binding fragment thereof comprises the light chain CDR1 comprising/consisting of the amino acid sequence RASQDISNYLN (SEQ ID NO:64), the light chain CDR2 comprising/consisting of the amino acid sequence FTSRLRS (SEQ ID NO:65); and the light chain CDR3 comprising/consisting of the amino acid sequence QQDRKLPWT (SEQ ID NO:66).

In one aspect, the application discloses an isolated antibody or antigen-binding fragment thereof that selectively binds to human CD40, wherein the heavy chain CDR1 comprises/consists of the amino acid sequence TFPIE (SEQ ID NO: 61); the heavy chain CDR2 comprises/consists of the amino acid sequence NFHPYNDDTKYNEKFKG (SEQ ID NO:62); the heavy chain CDR3 comprises/consists of the amino acid sequence RGKLPFDS (SEQ ID NO:63); the light chain CDR1 comprises/consists of the amino acid sequence RASQDISNYLN (SEQ ID NO:64); the light chain CDR2 comprises/consists of the amino acid sequence FTSRLRS (SEQ ID NO:65); and the light chain CDR3 comprises/consists of the amino acid sequence QQDRKLPWT (SEQ ID NO:66).

In another aspect, the application provides an isolated antibody or antigen-binding fragment thereof that selectively binds to human CD40 and comprises a variable heavy (VH) domain that is at least 80% identical to the amino acid sequence of SEQ ID NO:33.

In certain embodiments of this aspect, the VH domain is at least 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:33. In a specific embodiment, such a VH domain comprises the heavy chain CDR1 comprising/consisting of the amino acid sequence TFPIE (SEQ ID NO: 61); the heavy chain CDR2 comprising/consisting of the amino acid sequence NFHPYNDDTKYNEKFKG (SEQ ID NO:62); and the heavy chain CDR3 comprising/consisting of the amino acid sequence RGKLPFDS (SEQ ID NO:63)). In some embodiments, the antibody comprises a heavy chain comprising amino acids 21-463 of SEQ ID NO:46. In certain embodiments, the antibody or antigen-binding fragment thereof comprises a variable light (VL) domain that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO:34. In a specific embodiment, such a VL domain comprises a light chain CDR1 comprising/consisting of the amino acid sequence RASQDISNYLN (SEQ ID NO:64); a light chain CDR2 comprising/consisting of the amino acid sequence FTSRLRS (SEQ ID NO:65); and a light chain CDR3 comprising/consisting of the amino acid sequence QQDRKLPWT (SEQ ID NO:66)). In some embodiments, the VH domain is at least 90%, 91%, 92%, 93%, 94%, 95%, 96%, 97%, 98%, 99%, or 100% identical to the amino acid sequence of SEQ ID NO:33; and the VL domain is at least 90%, 91%, 92%, 93%, 94%, 95%, 96%, 97%, 98%, 99%, or 100% identical to the amino acid sequence of SEQ ID NO:34. In a specific embodiment, such a VH domain comprises a heavy chain CDR1 comprising/consisting of the amino acid sequence TFPIE (SEQ ID NO: 61); a heavy chain CDR2 comprising/consisting of the amino acid sequence NFHPYNDDTKYNEKFKG (SEQ ID NO:62); a heavy chain CDR3 comprising/consisting of the amino acid sequence RGKLPFDS (SEQ ID NO:63); and such a VL domain comprises a light chain CDR1 comprising/consisting of the amino acid sequence RASQDISNYLN (SEQ ID NO:64); a light chain CDR2 comprising/consisting of the amino acid sequence FTSRLRS (SEQ ID NO:65); and a light chain CDR3 comprising/consisting of the amino acid sequence QQDRKLPWT (SEQ ID NO:66)). In a specific embodiment, the heavy chain comprises amino acids 21-463 of SEQ ID NO:46 and the light chain comprises amino acids 23-236 of SEQ ID NO:38.

These embodiments apply to all of the above aspects. In certain instances, the antibody is a humanized antibody. In some embodiments, the antibody is a monoclonal antibody. In some instances, the antibody is a single chain antibody. In certain instances, the antibody is a polyclonal antibody, a chimeric antibody, an Fab fragment, an F(ab′)2 fragment, an Fab′ fragment, an Fsc fragment, an Fv fragment, an scFv, an sc(Fv)2, or a diabody. In some embodiments, the antibody has an IgG4 heavy chain constant region. In certain embodiments, wherein the antibody has an IgG4 heavy chain constant region, the antibody has a serine to proline mutation at position 228 (Kabat numbering, S228P) in the hinge region of the antibody. In some embodiments, the heavy chain of the antibody is glycosylated. In certain instances, the isolated antibody has reduced afucose content (e.g., 0.1% to 1.5% afucose). In some instances, the isolated antibody has reduced galactose content and/or reduced high mannose content compared to reference anti-CD40 antibodies. In certain instances, the antibody is a monovalent antibody fragment comprising a single target molecule (human CD40) binding arm and an Fc region (i.e., a complex of Fc polypeptides). In some embodiments, the monovalent antibody fragment is more stable in vivo than the monovalent antibody fragment comprising a single human CD40 binding arm without an Fc region. The single target molecule binding am can comprise the VH and VL CDRs, or a VH and VL region, of any of the anti-CD40 antibodies described herein (e.g., Exemplary anti-CD40 Antibody 1). In certain embodiments, the single target molecule binding arm is a scFv. In some embodiments, the single target molecule binding arm comprises two separate polypeptide chains, wherein the first polypeptide chain comprises a VH region of any of the anti-CD40 antibodies described herein (e.g., Exemplary anti-CD40 Antibody 1) and the second polypeptide chain comprises the VL region of any of the anti-CD40 antibodies described herein (e.g., Exemplary anti-CD40 Antibody 1). In specific embodiments, the first polypeptide chain comprises a VII region of any of the anti-CD40 antibodies described herein (e.g., Exemplary anti-CD40 Antibody 1) and a heavy chain (CH1) domain and the second polypeptide chain comprises the VL region of any of the anti-CD40 antibodies described herein (e.g., Exemplary anti-CD40 Antibody 1) and a light chain constant (CL) domain. The Fc region of the monovalent antibody fragment comprises a complex of a first and second Fc polypeptide, wherein one but not both of the Fc polypeptides is an N-terminally truncated heavy chain. In one embodiment, an N-terminally truncated heavy chain consists or consists essentially of a hinge sequence contiguously linked to a heavy chain CH2 domain (or a portion thereof) and/or a heavy chain CH3 domain (or a portion thereof) sufficient to form a complex with the first Fc polypeptide. In one embodiment, the N-terminally truncated heavy chain is of an IgG heavy chain (e.g., IgG1, IgG4). In another embodiment, both the first and second Fc polypeptide are of an IgG heavy chain (e.g., IgG1, IgG4). In certain embodiments, the Fc region has effector function that is the same as or less than that of Exemplary anti-CD40 Antibody 1. In certain embodiments, the monovalent antibody fragment comprises a proline at position 228 (Kabat numbering) in the hinge region of one or both Fc regions. In certain embodiments, the monovalent antibody fragment is linked/conjugated to polyethylene glycol (PEG), human serum albumin (HSA), or XTEN.

In certain embodiments, the antibodies disclosed herein have properties that make them clinically useful for treating a human subject in need of treatment with an anti-CD40 antibody. For example, the antibodies have one, two, three, four, five, or more of the following properties: (i) they are humanized to reduce immune responses against the antibody; (ii) the Fc region of the antibody is mutated from the wild type Fc so that the antibody has improved stability (e.g., S228P mutation); (iii) the Fc region has reduced effector function (e.g., IgG4 as compared to IgG1); (iv) has reduced agonism compared to chADH9 IgG1; (v) agonizes human CD40 at a level that is the same as or less than Exemplary anti-CD40 Antibody 1; (vi) can bind human CD40 on B cells in whole blood with the same or better affinity than Exemplary anti-CD40 Antibody 1; (vii) can fully inhibit CD40L-induced B cell activation; and (viii) can be formulated at high concentrations (e.g., 100 to 250 mg/mL) so that it can be administered subcutaneously. In some instances, the anti-CD40 antibodies disclosed herein are more effective than other anti-CD40 antibodies that are being considered for clinical use in a human subject. In certain instances, the anti-CD40 antibodies disclosed herein bind the CD40 receptor on cells better than the other CD40 antibodies while having a similar or lower agonism profile than other anti-CD40 antibodies that are being considered for clinical use in a human subject.

In another aspect, the anti-CD40 antibody is an IgG4P antibody (i.e., an antibody that has a proline at position 228 in the hinge region of IgG4 instead of a serine) comprising the VH CDR1, VH CDR2, and VH CDR3 of the humanized heavy chain variable region of AKH3. In certain embodiments, this anti-CD40 antibody further comprises the VL CDR1, VL CDR2, and VL CDR3 of the humanized light chain variable region of AKH3. In certain embodiments, the anti-CD40 antibody comprises an amino acid sequence that is 80%, 85%, 86%, 87%, 88%, 89%, 90%, 91%, 92%, 93%, 94%, 95%, 96%, 97%, 98%, 99%, or 100% identical to the sequence of SEQ ID NO:33. In some instances, this anti-CD40 antibody comprises the VH CDR1, VH CDR2, and VH CDR3 of the humanized heavy chain variable region of AKH3. In certain embodiments, the anti-CD40 antibody comprises an amino acid sequence that is 80%, 85%, 86%, 87%, 88%, 89%, 90%, 91%, 92%, 93%, 94%, 95%, 96%, 97%, 98%, 99%, or 100% identical to the sequence of SEQ ID NO:34. In some instances, this anti-CD40 antibody comprises the VL CDR1, VL CDR2, and VL CDR3 of the humanized heavy chain variable region of AKH3. In a specific embodiment, the anti-CD40 antibody comprises an amino acid sequence that is 80%, 85%, 86%, 87%, 88%, 89%, 90%, 91%, 92%, 93%, 94%, 95%, 96%, 97%, 98%, 99%, or 100% identical to the sequence of SEQ ID NO:33 and an amino acid sequence that is 80%, 85%, 86%, 87%, 88%, 89%, 90%, 91%, 92%, 93%, 94%, 95%, 96%, 97%, 98%, 99%, or 100% identical to the sequence of SEQ ID NO:34. In some instances, this anti-CD40 antibody comprises the VH CDR1, VH CDR2, and VH CDR3 and the VL CDR1, VL CDR2, and VL CDR3 of the humanized heavy chain variable region of AKH3. In some embodiments, the anti-CD40 antibody is humanized. In some embodiments, the anti-CD40 antibody is a monovalent antibody binding fragment.

In another aspect, the disclosure provides a nucleic acid encoding any of the antibodies or antigen-binding fragments thereof described herein.

In a further aspect, the application describes an isolated cell that produces any of the antibodies or antigen-binding fragments thereof described herein.

In yet another aspect, the disclosure provides a pharmaceutical composition comprising any of the antibodies or antigen-binding fragments described herein. In some instances, the antibody or antigen-binding fragment thereof are formulated in a composition comprising citrate buffer with arginine and having a pH of 5.5-6.5. In other instances, the antibody or antigen-binding fragment thereof are formulated in a composition comprising histidine buffer with arginine and having a pH of 5.5-6.5. In certain instances, pharmaceutical composition also includes sucrose, methionine, or polysorbate-80.

In another aspect, the application provides a method of inhibiting hyperactivation of B or T cells in a human subject in need thereof, the method comprising administering to the subject in need thereof an effective amount of any of the antibodies or antigen-binding fragments described herein.

In a different aspect, the disclosure provides a method of treating or preventing an autoimmune disease in a human subject in need thereof, the method comprising administering to the subject in need thereof an effective amount of any of the antibodies or antigen-binding fragments described herein. In certain instances, the autoimmune disease is one of Sjogren's syndrome, systemic lupus erythematosus, lupus nephritis, discoid lupus, acquired hemophilia, systemic sclerosis (scleroderma), Crohn's disease, ulcerative colitis, Graves disease, immune thrombocytopenic purpura, rheumatoid arthritis, asthma, vasculitis, pemphigoid, atopic dermatitis, or hemolytic anemia. In one embodiment, the autoimmune disease is Sjogren's syndrome. In another embodiment, the autoimmune disease is systemic lupus erythematosus. In another embodiment, the autoimmune disease is scleroderma. In yet another embodiment, the autoimmune disease is immune thrombocytopenic purpura.

In one aspect, the disclosure provides a method of treating or preventing transplant rejection in a human subject in need thereof, the method comprising administering to the subject in need thereof an effective amount of any of the antibodies or antigen-binding fragments described herein. In certain instances, the transplant rejection is induced after kidney transplantation, heart transplantation, liver transplantation, pancreas transplantation, intestine transplantation, or xenograft.

In yet another aspect, the disclosure provides a method of treating or preventing graft versus host disease in a human subject in need thereof, comprising administering to the subject in need thereof an effective amount of any of the antibodies or antigen-binding fragments thereof described herein.

In another aspect, the application discloses a method of treating or preventing Alzheimer's disease in a human subject in need thereof, the method comprising administering to the subject in need thereof an effective amount of any of the antibodies or antigen-binding fragments described herein.

In another aspect, the disclosure provides a method of treating or preventing neuromyelitis optica in a human subject in need thereof, the method comprising administering to the subject in need thereof an effective amount of any of the antibodies or antigen-binding fragments f described herein.

In a further aspect, the disclosure provides a method of treating or preventing myasthenia gravis in a human subject in need thereof, the method comprising administering to the subject in need thereof an effective amount of any of the antibodies or antigen-binding fragments described herein.

In a yet further aspect, the disclosure provides a method of treating or preventing amyotrophic lateral sclerosis in a human subject in need thereof, the method comprising administering to the subject in need thereof an effective amount of any of the antibodies or antigen-binding fragments thereof described herein.

In another aspect, the disclosure provides a method of treating or preventing hemophilia with inhibitors in a human subject in need thereof, the method comprising administering to the subject in need thereof an effective amount of any of the antibodies or antigen-binding fragments thereof described herein.

### BRIEF DESCRIPTION OF THE DRAWINGS

FIG. 1 is a graphical depiction of the binding of humanized H1L1 agly IgG4P/IgG1 to stably transfected CHO cells expressing human CD40 compared to the binding of chAKH3 IgG1 containing the original murine variable domains.

FIG. 2 is a graphical depiction of the binding and disassociation (determined by Octet) of human monomeric CD40 to humanized H1/L1 agly IgG4P/IgG1 and chimeric AKH3. Humanized H1L1 agly IgG4P/IgG1 and chAKH3 IgG1 containing the original murine variable domains were immobilized onto anti-human Fc Octet sensor tips to evaluate the binding kinetics (association and dissociation) of monomeric soluble CD40.

FIG. 3 is a schematic depiction of the plasmid map of BM098 encoding the humanized AKH3 H1 IgG4P heavy chain.

FIG. 4 is a schematic depiction of the plasmid map of BM099 encoding the humanized AKH3 L1 light chain.

FIG. 5 provides a series of sensograms generated from solid phase affinity measurements for Exemplary anti-CD40 Antibody 1 Fab fragment binding to human, cynomolgus or rhesus Fc-CD40 fusion proteins shown as response versus time over a 0.15 nM-1.5 nM Fab concentration range.

FIG. 6 is a series of graphs depicting AKH3 binding to cell surface CD40. AKH3 binding to CD40 on CHO cells stably expressing human, cynomolgus, or rhesus CD40 as measured by flow cytometry, with mean fluorescence intensity (MFI) normalized to the maximal signal (% max MFI). The agly hAKH3 IgG4P/IgG1 mAb which has a V region identical to that of Exemplary anti-CD40 Antibody 1 was employed (top) as were mAKH3 Fab fragments.

FIG. 7 is a bar graph depicting AKH3 mAb binding to cell surface human and rodent CD40. mAKH3 mAb binding to 293E transiently transfected cells expressing CD40 of mouse, rat or human or untransfected cells (negative control), shown as the Mean Fluorescence Intensity (MFI) value for 1000 or 100 ng/mL mAb.

FIG. 8 provides a series of graphs depicting A647-conjugated AKH3 binding to B cells in whole blood. The agly hAKH3 IgG4P/IgG1 mAb which has a V region identical to that of Exemplary anti-CD40 Antibody 1 was employed to determine the EC₅₀ of binding. Representative results are shown as the geometric mean fluorescence intensity (A647 Geomean) versus mAb concentration for two normal human donors and for the direct comparison of binding in humans and cynomolgus monkeys (right).

FIG. 9 is a scatter graph of the EC₅₀ values obtained for binding of the A647-fluorochrome conjugated agly hAKH3 IgG4P/IgG1 to B cells in human and cynomolgus monkey whole blood. EC₅₀ values were derived from binding curves of the flow cytometry measurement (A647 geometric mean fluorescence intensity) versus mAb concentration for each of 7 individual humans and 9 individual cynomolgus monkeys. The agly hAKH3 IgG4P/IgG1 mAb has a V region identical to that of Exemplary anti-CD40 Antibody 1.

FIG. 10 are graphical representations of mAKH3 mAb inhibition of rsCD40L (1 μg/mL) binding to RAMOS B cells shown as the mean fluorescence intensity of the biotinylated sCD40L detected by APC-conjugated streptavidin (SA APC) over a dose range of mAb. Inhibition curves and EC₅₀ values are shown for two independent determinations.

FIG. 11 are co-crystal structures for (left image) the mAKH3 Fab fragment (ribbon diagram with Heavy chain and Light chain with human CD40 (4 domains-CRD1 through CRD4) and (right image) human rsCD40L (space filled structure) and human CD40.

FIG. 12 provides a series of graphs showing the functional potency of Exemplary anti-CD40 Antibody 1 for inhibition of recombinant soluble human CD40 ligand (rsCD40L)-induced B cell activation in human whole blood. The results are shown as the geometric mean fluorescence of the CD69 activation marker measured by flow cytometry over a range of Exemplary anti-CD40 Antibody 1 concentrations. Representative data are shown for normal healthy donors (BIIB donors, top), SLE patients (middle) and RA patients (bottom). Reference anti-CD40 Antibody in this figure corresponds to Reference Ab 1 (IgG4P).

FIG. 13 is a scatter graph of the IC₅₀ values obtained for the functional potency of Exemplary anti-CD40 Antibody 1 in whole blood cultures from normal, SLE and RA donors, as measured by Exemplary anti-CD40 Antibody 1 inhibition of expression of the CD69 activation marker on B cells by flow cytometry for each of 8 normal, 5 SLE and 6 RA individual donors. Geometric mean values for each cohort are indicated.

FIG. 14 is a graphical depiction of the functional potency of Exemplary anti-CD40 Antibody 1 for inhibition of rsCD40L-induced B cell activation in cynomolgus monkey and human whole blood. The results are shown as the geometric mean fluorescence of the CD95 activation marker measured by flow cytometry over a range of Exemplary anti-CD40 Antibody 1 concentrations. Representative data are shown for cynomolgus monkeys and normal healthy human donors. Reference anti-CD40 Antibody in this figure corresponds to Reference Ab 1 (IgG4P).

FIG. 15 is a scatter graph of the IC₅₀ values obtained for the functional potency of Exemplary anti-CD40 Antibody 1 in whole blood cultures from cynomolgus monkeys and normal human donors, as measured by Exemplary anti-CD40 Antibody 1 inhibition of expression of the CD95 activation marker on B cells by flow cytometry for each of 5 cyno and 3 normal human individuals. Geometric mean values for each cohort are indicated.

FIG. 16 is a bar graph illustrating that Exemplary anti-CD40 Antibody 1 is minimally agonistic in a RAMOS B cell line. Ramos-Blue NF-κB/AP-1 reporter cell line was cultured with varying concentrations of anti-CD40 mAbs or polyclonal human IgG. NF-κB induced alkaline phosphatase secretion was measured by combining conditioned cell culture media with an alkaline phosphatase substrate. Results shown represent the fold increase over baseline (cells only) of the optical density (OD) 620 nm readings. Reference anti-CD40 Antibody in this figure corresponds to Reference Ab 1 (IgG4P).

FIG. 17 provides a series of graphs showing that Exemplary anti-CD40 Antibody 1 is minimally agonistic in human B cell and DC cultures. B cells isolated from peripheral blood of a normal healthy donor were cultured in the presence of polyclonal anti-IgM and various concentrations of anti-CD40 mAbs overnight. B cell activation marker ICAM-1 (CD54) expression was measured by flow cytometry and results shown as the geometric mean fluorescence (left). Monocytes isolated were matured into DC by standard methods and cultured in the presence of IFNγ and various concentrations of anti-CD40 mAbs for 48 hrs. DC activation marker, CD86 expression, was measured by flow cytometry and results shown as geometric mean fluorescence (right). Reference anti-CD40 Antibody in this figure corresponds to Reference Ab 1 (IgG4P).

FIG. 18 is a series of graphs showing that Exemplary anti-CD40 Antibody 1 is minimally agonistic in human whole blood cultures. Whole blood cultures from human normal donors, SLE and RA patients were exposed to anti-CD40 mAbs in the presence of IL-4 and results shown as the geometric mean fluorescence of the CD69 activation marker measured by flow cytometry. Representative data are shown for normal healthy donors (top), SLE patients (middle) and RA patients (bottom). Reference anti-CD40 Antibody in this figure corresponds to Reference Ab 1 (IgG4P).

FIG. 19 provides a summary of agonist activity assessment in human whole blood cultures from normal healthy donors and autoimmune disease patients. Results of agonism assays in whole blood from human normal healthy donors, SLE patients, and RA patients, are shown as the fold change in the geometric mean fluorescence of the CD69 activation marker for anti-CD40 mAb in the presence of IL-4 over that of IL-4 alone. Individual points on the scatter plots indicate the highest fold increase observed for the anti-CD40 mAb titration over baseline in a given assay. Horizontal bars indicate the mean values. ADH9 is consistently agonistic and Exemplary anti-CD40 Antibody 1 only minimally agonistic for B cell activation in human whole blood cultures. Reference anti-CD40 Antibody in this figure corresponds to Reference Ab 1 (IgG4P).

FIG. 20 are correlation plots of RF value for each of 8 individual RA patients versus corresponding result in the agonism assay, reported as fold change in the B cell activation marker CD69 measured by the geometric mean fluorescence intensity in whole blood cultures with Exemplary anti-CD40 Antibody 1 in the presence of IL-4 over that with IL-4 alone. There was no significant correlation for the Exemplary anti-CD40 Antibody 1 or the chADH9 IgG4P positive control mAb. Similar results were obtained for a Reference anti-CD40 antibody 1, IgG4 (data not shown).

FIG. 21 provides a correlation analysis of CD69 and CD95 readouts in human whole blood agonism assays. Representative results are shown for three individual human donors.

FIG. 22 is a series of graphs showing that Exemplary anti-CD40 Antibody 1 is minimally agonistic in cynomolgus monkey whole blood cultures. Whole blood cultures from cynomolgus monkeys and normal human controls were exposed to anti-CD40 mAbs over a dose range of 1-50 ng/mL in the presence of human IL-4 and results shown as the geometric mean fluorescence of the B cell activation marker CD95 expression by flow cytometry with an anti-CD95 PerCp Cy5.5 antibody conjugate. Reference anti-CD40 Antibody in this figure corresponds to Reference Ab 1 (IgG4P).

FIG. 23 provides a summary of agonist activity assessment showing comparability between human and cynomolgus monkey whole blood cultures. Results of the agonism assays in whole blood from cynomolgus monkey donors is shown as the fold change in the geometric mean fluorescence of the CD95 activation marker for anti-CD40 mAb in the presence of IL-4 over that of IL-4 alone. Individual points on the scatter plot indicate the highest fold increase observed for the anti-CD40 mAb titration over baseline in a given assay. ADH9 is consistently agonistic and Exemplary anti-CD40 Antibody 1 only minimally agonistic for B cell activation in cynomolgus monkey whole blood cultures. Reference anti-CD40 Antibody in this figure corresponds to Reference Ab 1 (IgG4P).

FIG. 24 is a bar graph illustrating that AKH3 is minimally agonistic in platelet cultures. Sepharose gel-filtered platelets were exposed to 100 μg/mL of anti-CD40 mAbs in a quiescent or sub-optimally activated state (treatment with 2 μM ADP, 20 μg/mL rsCD40L, or a combination of the two). Platelet activation was assessed by CD62-P (P-selectin) expression using flow cytometry. Reference anti-CD40 Antibody in this figure corresponds to Reference Ab 1 (IgG4P).

FIG. 25 is a bar graph showing that anti-CD40L mAb hu5c8 is agonistic in platelet cultures. Platelet-rich plasma, in either a quiescent or sub-optimally activated state (treatment with 20 μg/mL rsCD40L, 2 μM ADP, or a combination of the two) was exposed to 100 μg/mL of anti-CD40L antibody human 5c8 for 30 minutes at 37° C. Platelet activation was assessed by CD62-P (P-selectin) expression using flow cytometry.

FIG. 26 is a series of graphs showing reduced Binding of Exemplary anti-CD40 Antibody 1 to human FcγR CD16a V158, CD32a R131, CD32b, and CD64 as compared to a reference anti-CD40 antibody, fully Fc-competent WT IgG1, (chAKH3 IgG1) and a negative control, Fc-effectorless agly IgGP/G1(agly chAKH3), by ALPHAscreen technology. Exemplary anti-CD40 Antibody 1 exhibits reduced binding to all of the FcγR as compared to WT IgG1.

FIG. 27 is a graph showing that Exemplary anti-CD40 Antibody 1 is devoid of C1q binding activity. C1q binding of chAKH3 IgG1 but not Exemplary anti-CD40 Antibody 1 or an Fc-effectorless construct, agly chAKH3 determined by ELISA.

FIG. 28 provides the results of agonism assays in human whole blood from nine normal healthy donors and eight SLE donors, shown as the fold change in the geometric mean fluorescence of the CD69 activation marker for anti-CD40 construct in the presence of IL-4 over that of IL-4 alone. Individual points on the scatter plots indicate the highest fold increase observed for the anti-CD40 mAb titration over baseline in a given assay. Horizontal bars indicate the mean values. ADH9, the positive control anti-CD40 antibody is consistently agonistic regardless of whether the Fc region scaffold is huIgG1, IgG4P or agly IgG4P/IgG1. hAKH3 IgG4P (Exemplary anti-CD40 Antibody 1) is minimally agonistic for B cell activation in the human whole blood cultures as compared to the agly hAKH3 IgG4P/IgG1 construct. Reference anti-CD40 Antibody in this figure corresponds to Reference Ab 1 (IgG4P).

FIG. 29 provides a series of graphs depicting Exemplary anti-CD40 Antibody 1 dose-dependent inhibition of the anti-TT antibody response. Kinetics of serum anti-TT IgG antibodies in cynomolgus monkeys dosed IV with vehicle or Exemplary anti-CD40 Antibody 1 at 1, 3, 10 or 30 mg/kg on day 0, followed by TT by IM route at 4 hours post dose. Each line represents the serum titer in an individual cynomolgus monkey, with 5 monkeys per dose group.

FIG. 30 provides bar graphs showing the area under the curve (AUC) and percent inhibition in Exemplary anti-CD40 Antibody 1-dosed cynomolgus monkeys. Serum anti-TT AUC values (A, upper graph) and % inhibition values (B, upper graph) for individual cynomolgus monkeys dosed with vehicle (c1501-1505), Exemplary anti-CD40 Antibody 1 1 mg/kg (c2501-2505), 3 mg/kg (c3501-3505), 10 mg/kg (c4501-4505) and 30 mg/kg (c5501-5505). Lower graphs show the group averages for AUC and % inhibition. The percent inhibition was calculated as compared to the average AUC for the vehicle treated group.

FIG. 31 is a series of graphs showing CD40 receptor occupancy kinetics in whole blood of cynomolgus monkeys dosed with Exemplary anti-CD40 Antibody 1 or vehicle control. Occupancy of CD40 on the surface of cynomolgus monkey peripheral blood CD45⁺CD20⁺ B cells in cynomolgus monkeys dosed with vehicle or Exemplary anti-CD40 Antibody 1 at 1, 3, 10, and 30 mg/kg on day 0. Individual cynomolgus monkeys are represented by a single line in each dosing group. All staining was performed on 100 μl of fresh venous whole blood, in the dark, on ice. The average CD40 expression from two pre-bleeds performed prior to the dosage of Exemplary anti-CD40 Antibody 1 or vehicle control was used to normalize each cynomolgus monkey (“baseline”). CD40 receptor occupancy is demonstrated for each dosing group using Alexa647-Exemplary anti-CD40 Antibody 1 labeled antibody (left y-axis, black lines and symbols). Total CD40 on the B cell surface is demonstrated using Alexa488-PGN labeled antibody (right y-axis, grey lines and symbols).

FIG. 32 is a series of graphs depicting the percentage of circulating B Cells in whole blood of cynomolgus monkeys dosed with Exemplary anti-CD40 Antibody 1 or vehicle control. Circulating B cells (CD20⁺ cells) as a percentage of baseline in cynomolgus monkeys dosed with vehicle or Exemplary anti-CD40 Antibody 1 at 1, 3, 10, and 30 mg/kg on day 0. Individual cynos are represented by a single line in each dosing group. Whole blood from cynomolgus monkeys was stained with an immunofluorescent cocktail to identify CD45⁺CD20⁺ B cells. The average percent CD20±cells from two pre-bleeds performed prior to the dosage of Exemplary anti-CD40 Antibody 1 or vehicle control was used to normalize each cynomolgus monkey (“baseline”).

FIG. 33 is a series of graphs showing absolute lymphocyte count in whole blood of cynomolgus monkeys dosed with Exemplary anti-CD40 Antibody 1 or vehicle control. Absolute lymphocyte counts shown as the % of baseline in cynomolgus monkeys dosed with vehicle or Exemplary anti-CD40 Antibody 1 at 1, 3, 10, and 30 mg/kg on day 0. Individual cynomolgus monkeys are represented by a single line in each dose group. Venous whole blood was collected in EDTA tubes, and hematology analysis was performed on an Advia 120/2120 system. The average lymphocyte count from two pre-bleeds performed prior to the dosage of Exemplary anti-CD40 Antibody 1 or vehicle control was used to normalize each cynomolgus monkey (“baseline”).

FIG. 34 is a series of graphs showing the expression of the CD86 activation marker on the surface of B Cells in cynomolgus monkeys dosed with Exemplary anti-CD40 Antibody 1 or vehicle control. CD86 expression on B cells (geometric mean) from cynomolgus monkeys dosed with vehicle or Exemplary anti-CD40 Antibody 1 at 1, 3, 10, and 30 mg/kg. Whole blood from cynomolgus monkeys was stained with an immunofluorescent cocktail to identify CD86 expression on the surface of circulating CD45⁺CD20⁺ B cells. Individual cynomolgus monkeys are represented by a single line in each dosing group. All values are expressed relative to the median value of all predosing samples available (2/monkey×25 monkeys). Dotted lines indicate the 95% confidence intervals for the median.

FIG. 35 is a series of graphs showing the expression of the CD95 activation marker on the surface of B Cells in cynomolgus monkeys dosed with Exemplary anti-CD40 Antibody 1 or vehicle control. CD95 expression on B cells (geometric mean) from cynomolgus monkeys dosed with vehicle or Exemplary anti-CD40 Antibody 1 at 1, 3, 10, and 30 mg/kg. Whole blood from cynomolgus monkeys was stained with an immunofluorescent cocktail to identify CD95 expression on the surface of circulating CD45⁺CD20⁺ B cells. Each line represents an individual cynomolgus monkeys in each dosing group. All values are expressed relative to the median value of all predosing samples available (2/monkey×25 monkeys). Dotted lines indicate the 95% confidence intervals for the median.

FIG. 36 is a series of graphs showing serum IL-12 levels in cynomolgus monkeys dosed with Exemplary anti-CD40 Antibody 1 or vehicle control. Serum IL-12 levels were assessed using a custom 16-plex magnetic bead kit (Life Technologies) and data acquired using the Luminex 200 platform. Each line represents an individual cynomolgus monkey in each dosing group. All values are expressed relative to the median value of all predosing samples available (2/monkey×25 monkeys). Dotted lines indicate the 95% confidence intervals for the median.

FIG. 37 is a series of graphs showing serum IFNγ levels in cynomolgus monkeys dosed with Exemplary anti-CD40 Antibody 1 or vehicle control. Serum IFNγ levels were assessed in using a custom 16-plex magnetic bead kit (Life Technologies) and data acquired using the Luminex 200 platform. Each line represents an individual cynomolgus monkey in each dosing group. All values are expressed relative to the median value of all predosing samples available (2/monkey×25 monkeys). Dotted lines indicate the 95% confidence intervals for the median.

FIG. 38 is a series of graphs showing serum IL-6 levels in cynomolgus monkeys dosed with Exemplary anti-CD40 Antibody 1 or vehicle control. Serum IFNγ levels were assessed in using a custom 16-plea magnetic bead kit (Life Technologies) and data acquired using the Luminex 200 platform. Each line represents an individual cynomolgus monkey in each dosing group. All values are expressed relative to the median value of all predosing samples available (2/monkey×25 monkeys). Dotted lines indicate the 95% confidence intervals for the median.

FIG. 39 is a series of graphs showing serum TNF-α levels in cynomolgus monkeys dosed with Exemplary anti-CD40 Antibody 1 or vehicle control. Serum IFNγ levels were assessed in using a custom 16-plex magnetic bead kit (Life Technologies) and data acquired using the Luminex 200 platform. Each line represents an individual cynomolgus monkey in each dosing group. All values are expressed relative to the median value of all predosing samples available (2/monkey×25 monkeys). Dotted lines indicate the 95% confidence intervals for the median.

FIG. 40 is a series of graphs showing the PK/PD relationship of serum Exemplary anti-CD40 Antibody 1 levels and CD40 Receptor occupancy in cynomolgus monkeys. CD40 receptor occupancy shown in solid lines, overlaid with serum Exemplary anti-CD40 Antibody 1 drug levels in dashed lines, demonstrating an exposure-efficacy relationship where CD40 occupancy is correlated with serum Exemplary anti-CD40 Antibody 1 levels. Each line represents an individual cynomolgus monkey in each dosing group. Serum samples that were BLQ in the PK ELISA were plotted as 0.01 μg/mL.

FIG. 41 is a bar graph illustrating that mAKH3 selectively binds to human CD40. Human TNF superfamily receptors expressed as human Fc fusion proteins were immobilized and the binding of 5 μg/mL of mAKH3 was detected with anti-mouse IgG HRP (hatched bars). Anti-human IgG HRP was used to evaluate the coating density of the TNF receptor-Fc fusions proteins (filled in bars).

FIG. 42A is a structure depicting the mAKH3 epitope (amino acid residues in black) on the CD40 ECD (left structure). The differences in cynomolgus or rhesus monkey as compared to human CD40 ECD, 6 for cynomolgus vs. human and an additional residue for rhesus vs. human are also shown (right structure). A dotted circle indicates the location of the T112M mutation, which is only found in Rhesus CD40. It is the only site that truly overlaps with the AKH3 epitope. The L121P mutation in rhesus CD40 is not anticipated to clash with the AKH3 paratope because it is at the periphery and because Pro is smaller than Leu. FIG. 42B shows AKH3 binding to human CD40 ECD and that the location in rhesus CD40 ECD of a methionine at position 112 would clash with AKH3 binding to rhesus CD40 ECD.

FIG. 43 is a series of graphs for four individual human donors, each showing the functional potency of Exemplary anti-CD40 Antibody 1 as compared to other anti-CD40 antibodies for inhibition of rsCD40L-induced B cell activation in human whole blood. The results are shown as the geometric mean fluorescence of the CD54 activation marker measured by flow cytometry over a range of anti-CD40 antibody concentrations.

FIG. 44 is a summary of agonist activity assessment in human whole blood cultures from normal healthy donors shown as the fold change in the geometric mean fluorescence of the CD69 activation marker for anti-CD40 antibody in the presence of IL-4 over that of IL-4 alone. Individual points on the scatter plots correspond to individual donors and indicate the highest fold increase observed for the anti-CD40 antibody titration over baseline in a given assay. Horizontal bars indicate the mean values.

FIG. 45 is a graph displaying results of experiments conducted to dissect if the agonistic activity observed with the aglycosyl IgG4P/IgG1 constructs was caused by removal of the N-linked glycans or the addition of the IgG1 CH3 domain.

### DETAILED DESCRIPTION

The antibodies described herein specifically bind to human CD40 and inhibit the interaction between CD40 and its ligand, CD40L. These antibodies exhibit reduced agonistic activity in whole blood cultures compared to other anti-CD40 antibodies while maintaining formation of desired antibody dimers; can inhibit the humoral response to tetanus toxoid immunization in a primate without B cell depletion compared to vehicle; not elevate IL-12 serum levels in a primate compared to an appropriate control; can bind to a CD40 protein encoded by a DNA molecule that contains the human CD40 SNP C77F about 50% as well as to wild type CD40 protein (SEQ ID NO:58); and can bind to a protein encoded by a DNA molecule that contains the human CD40 H78Q comparably as to wild type CD40 protein (SEQ ID NO:58). The antibodies with the properties described herein were identified after a long and dedicated search that involved screening over 141 hybridoma clones as well as multiple rounds of panning phage display libraries.

### CD40

CD40 is a Type I transmembrane receptor that is constitutively expressed by B cells, macrophages, dendritic cells, and other hematopoietic cells as well as non-hematopoietic cell types, including platelets, epithelial, endothelial, and stromal cells. The engagement of CD40 by its ligand, CD40 ligand (CD40L), also known as CD154, constitutes a key axis for the activation of innate and adaptive immune functions. The functional outcomes of CD40 engagement in different cell types are tabulated below.

| Cell Type | Functional Outcomes of CD40 Signaling |
|---|---|
| B cells | Activation: upregulation of antigen presentation and costimulatory molecules |
| | (MHC Class II, CD80, CD86), CD23, CD30, Fas/CD95, CD69, CD54 and |
| | cytokine production (IL-2, IL-6, IL-10, TNF-α, TGF-β) |
| | Clonal expansion and differentiation to antibody forming cells |
| | Generation of memory cells |
| | Primary and secondary antibody responses |
| | Isotype class switch (IgG, IgA, IgE) |
| | Germinal Center formation and maintenance |
| T cells | Activation: upregulation of CD25 expression |
| | Proliferation |
| | Cytokine production |
| | Optimal T helper responses (Th1, Th2, Tfh, Th17) |
| Monocytes/ | Activation: upregulation of antigen presentation and costimulatory molecules |
| macrophages | (MHC Class II, CD80, CD86), and cytokine production (IL-1, IL-12, TNF-α), |
| | and chemokine production |
| | Nitric Oxide (NO) production |
| | Killing of intracellular pathogens |
| | Matrix metalloproteinase (MMP) production |
| | Procoagulant activity (Tissue factor expression) |
| DC | Activation; upregulation of antigen presentation and costimulatory molecules |
| | (MHC Class II, CD80, CD86), and cytokine production (IL-1, IL-12) |
| | Growth and survival |
| | Enhanced cytokine production |
| Follicular DC | Growth |
| | CD54 expression |
| Endothelial cells | Upregulation of adhesion molecules (CD54/ICAM, CD62E/E-selectin, |
| | CD106/VCAM) |
| | Chemokines |
| | Procoagulant activity (Tissue factor expression) |
| Epithelial cells | Cytokine/chemokine production (IL-6, IL-8, MCP-1 RANTES) |
| Stromal cells | Proliferation |
| | Cytokine/chemokine production |
| | Procoagulant activity (Tissue factor expression) |

Blocking CD40 can potentially reduce the above downstream effects of CD40 signaling; dampening the hyperactivation of adaptive and innate immune responses in patients e.g., with autoimmune and inflammatory diseases.

The amino acid sequence of the human CD40 protein (Genbank® Accession No. NP_001241) is shown below (the extracellular domain (P20 to R193) is underlined).

| (SEQ ID NO: 58) | |
|---|---|
| 1 | MVRLPLQCVL WGCLLTAVHP EPPTACREKQ YLINSQCCSL CQPGQKLVSD CTEFTETECL |
| 61 | PCGESEFLDT WNRETHCHQH KYCDPNLGLR VQQKGTSETD TICTCEEGWH CTSEACESCV |
| 121 | LHRSCSPGFG VKQIATGVSD TICEPCPVGF FSNVSSAFEK CHPWTSCETK DLVVQQAGTN |
| 181 | KTDVVCGPQD RLRALVVIPI IFGILFAILL VLVFIKKVAK KPTNKAPHPK QEPQEINFPD |
| 241 | DLPGSNTAAP VQETLHGCQP VTQEDGKESR ISVQERQ |

The amino acid sequence of cynomolgus CD40 protein (Genbank Accession No. XP_005569275) is shown below. Cysteine rich domain 1 (CRD1) is boldened; CRD2 is underlined; CRD3 is italicized; and CRD4 is both boldened and underlined. The cynomolgus CD40 protein is 93% identical to the human CD40 protein.

| (SEQ ID NO: 59) | |
|---|---|
| 1 | MVRLPLQCVL WGCLLTAVYP EPPTACREKQ YLINSQCCSL CQPGQKLVSD CTEFTETECL |
| 61 | PCGESEFLDT WNRETRCHQH KYCDPNLGLR VQQKGTSETD TICTCEEGLH CTSESCESCV |
| 121 | PHRSCLPGFG VKQIATGVSD TICEPCPVGF FSNVSSAFEK CRPWTSCETK DLVVQQAGTN |
| 181 | KTDVVCGPQD RQRALVVIPI CLGILFVILL LVLVFIKKVA KKPNDKVPHP KQEPQEINFP |
| 241 | DDLPGSNPAA PVQETLEGCQ PVTQEDGKES RISVQERQ |

The amino acid sequence of rhesus CD40 protein (Genbank® Accession No. EHH19629) has 92% identity to human CD40 and 99% identity to cynomolgus CD40, whereas the amino acid sequence of rat CD40 protein (Genbank® Accession No. XP_006235573) has 55% identity to human and cynomolgus CD40, and the amino acid sequence of mouse CD40 protein (Genbank® Accession No. AAB08705) has 61% identity to human and cynomolgus CD40.

The human or cynomolgus CD40 proteins can be used as immunogens to prepare anti-CD40 antibodies. To prepare anti-human CD40 antibodies, the human CD40 protein is used as the immunogen. Such anti-human CD40 antibodies can then be screened to identify antibodies having one or more of the features described herein (e.g., selective binding to an epitope within cysteine-rich domain 2 (CRD2) and cysteine-rich domain 3 (CRD3) of the extracellular domain of human and cynomolgus CD40; inhibiting the humoral response to tetanus toxoid immunization in a primate without B cell depletion compared to vehicle; not elevating IL-12 serum levels in a primate compared to vehicle; competing with Exemplary Anti-CD40 Antibody 1 to bind CD40; binding with high affinity (e.g., a KD≦3 nM (e.g., 0.1 nM-3 nM; 0.25 nM-3 nM; 0.5 nM-3 nM; 0.75 nM-3 nM; 1 nM-3 nM; 1.25 nM-3 nM; 1.5-3 nM; 2-3 nM; 2.25 nM-3 nM; 2.5-3 nM; 2.75-3 nM)) to human and/or cynomolgus CD40; having low effector activity; having low agonistic activity in whole blood assays; inhibiting B cell activation in whole blood by soluble CD40L with an IC50 of between 10 and 200 ng/mL; not agonizing platelets stimulated by soluble CD40L; having reduced binding as compared to a wild type IgG1 antibody to CD16a, CD32a, CD32b, and/or CD64; and binding to both the wild type human CD40 protein (SEQ ID NO:58) as well as a protein encoded by a human CD40 DNA sequence containing any of the following human CD40 SNPs: A25S; S124L; I134V; F158L; S166R; S65R; D69E; H78Q; H80R; R90W; I134L; I134T; and V138F (binding comparably as to wild type CD40).

### Anti-CD40 Antibodies

This disclosure provides anti-CD40 antibodies or antigen binding fragments thereof that can block the CD40/CD40L interaction and thus are useful in treating immunological diseases such as autoimmune disorders and inflammatory disorders. These antibodies all bind human CD40. Such anti-CD40 antibodies includes the sequences of an anti-CD40 monoclonal antibody, Exemplary Anti-CD40 Antibody 1, which binds with high affinity (e.g., KD≦3 nM (monovalent affinity) or KD≦10 pM (bivalent affinity)) to both human and cynomolgus CD40, with much lower affinity to rhesus CD40 (monovalent KD could not be measured by Biacore (no binding); bivalent binding is in nM range on cells), and has undetectable binding to mouse or rat CD40.

### Exemplary Anti-CD40 Antibody 1

Exemplary Anti-CD40 Antibody 1 is a humanized IgG4/kappa monoclonal antibody with serine at position 225 (S228 according to Kabat numbering) of the heavy chain hinge region changed to proline to avoid half antibody formation in vivo (IgG4P). It specifically binds human and cynomolgus CD40 with high affinity (KD≦3 nM) and has low effector functionality. Exemplary Anti-CD40 Antibody 1 was constructed from a murine antibody, AKH3. The AKH3 murine hybridoma was derived from an RBF mouse immunized with a complex of CD40/CD40L extracellular domain constructs. Splenocytes from one mouse were fused to FL653 myeloma cells resulting in a hybridoma that produced the AKH3 antibody, which bound to human CD40 and blocked its interaction with CD40L. The AKH3 antibody was humanized and engineered into an IgG4P framework to have low effector function.

The amino acid sequences of the mature Exemplary Anti-CD40 Antibody 1 heavy and light chains are shown below. Complementarity-determining regions (CDRs) 1, 2, and 3, according to Kabat, of the variable light chain (VL) and the variable heavy chain (VH) are shown in that order from the N to the C-terminus of the mature VL and VH sequences and are both underlined and boldened. An antibody consisting of the mature heavy chain (SEQ ID NO:39) and the mature light chain (SEQ ID NO:42) listed below is termed Exemplary Anti-CD40 Antibody 1.

### Mature Exemplary Anti-CD40 Antibody 1 Light Chain (LC)

| (SEQ ID NO: 42) | |
|---|---|
| 1 | DIQMTQSPSS LSASVGDRVT ISCRASQDIS NYLNWYQQKP GKVPKLLIYF |
| 51 | TSRLRSGVPS RFSGSGSGTD YTLTISSLQP EDVATYYCQQ DRKLPWTFGQ |
| 101 | GTKLEIKRTV AAPSVFIFPP SDEQLKSGTA SVVCLLNNFY PREAKVQWKV |
| 151 | DNALQSGNSQ ESVTEQDSKD STYSLSSTLT LSKADYEKHK VYACEVTHQG |
| 201 | LSSPVTKSFN RGEC |

### Mature Exemplary Anti-CD40 Antibody 1 Heavy Chain (HC) (Hinge Region Mutation is Highlighted)

| (SEQ ID NO: 39) | |
|---|---|
| 1 | EVQLVQSGAE VKKPGASVKV SCKASGYTFT TFPIEWVRQA PGQGLEWMGN |
| 51 | FHPYNDDTKY NEKFKGRVTL TADKSTSTAY MELSRLRSED TAVYYCARRG |
| 101 | KLPFDSWGQG TTVTVSSAST KGPSVFPLAP CSRSTSESTA ALGCLVKDYF |
| 151 | PEPVTVSWNS GALTSGVHTF PAVLQSSGLY SLSSVVTVPS SSLGTKTYTC |
| 201 | NVDHKPSNTK VDKRVESKYG PPCPPCPAPE FLGGPSVFLF PPKPKDTLMI |
| 251 | SRTPEVTCVV VDVSQEDPEV QFNWYVDGVE VHNAKTKPRE EQFNSTYRVV |
| 301 | SVLTVLHQDW LNGKEYKCKV SNKGLPSSIE KTISKAKGQP REPQVYTLPP |
| 351 | SQEEMTKNQV SLTCLVKGFY PSDIAVEWES NGQPENNYKT TPPVLDSDGS |
| 401 | FFLYSRLTVD KSRWQEGNVF SCSVMHEALH NHYTQKSLSL SLG |

The variable light chain (VL) of Exemplary Anti-CD40 Antibody 1 has the following amino acid sequence:

| (SEQ ID NO: 34) | |
|---|---|
| 1 | DIQMTQSPSS LSASVGDRVT ISCRASQDIS NYLNWIQQKP GKVPKLLIYF |
| 51 | TSRLRSGVPS RFSGSGSGTD YTLTISSLQP EDVATYYCQQ DRKLPWTFGQ |
| 101 | GTKLEIK |

The variable heavy chain (VH) of Exemplary Anti-CD40 Antibody 1 has the following amino acid sequence:

| (SEQ ID NO: 33) | |
|---|---|
| 1 | EVQLVQSGAE VKKPGASVKV SCKASGYTFT TFPIEWVRQA PGQGLEWMGN |
| 51 | FHPYNDDTKY NEKFKGRVTL TADKSTSTAY MELSRLRSED TAVYYCARRG |
| 101 | KLPFDSWGQG TTVTVSS |

The amino acid sequences of VL CDRs (according to Kabat) of Exemplary Anti-CD40 Antibody 1 comprise/consist of the sequences listed below:

VL CDR1:
(SEQ ID NO: 64)
RASQDISNYLN;
VL CDR2:
(SEQ ID NO: 65)
FTSRLRS;
and
VL CDR3:
(SEQ ID NO: 66)
QQDRKLPWT.

The amino acid sequences of the VH CDRs (according to Kabat) of Exemplary Anti-CD40 Antibody 1 comprise/consist of the sequences listed below:

VH CDR1:
(SEQ ID NO: 61)
TFPIE;
VH CDR2:
(SEQ ID NO: 62)
NFHPYNDDTKYNEKFKG;
and
VH CDR3:
(SEQ ID NO: 63)
RGKLPFDS

The anti-CD40 antibodies or antigen binding fragments thereof of this disclosure can also comprise or consist of “alternate CDRs” of Exemplary Anti-CD40 Antibody 1. By “alternate” CDRs are meant CDRs (CDR1, CDR2, and CDR3) defined according to a definition other than Kabat such as, but not limited to, Chothia (e.g., Chothia from Abysis); enhanced Chothia/AbM CDR; or the contact definitions. These alternate CDRs can be determined, e.g., by using the AbYsis database (www.bioinf.org.uk/abysis/sequence_input/key_annotation/key_annotation.cgi). The amino acid sequences of “alternate” CDRs 1, 2, and 3 of the heavy chain variable region and the light chain variable region of Exemplary Anti-CD40 Antibody 1 are compared with the CDRs defined according to Kabat in the Table below.

| | | Chothia, | Enhanced | |
|---|---|---|---|---|
| Domain | Kabat | from Abysis | Chothia/AbM | Contact |
| VH CDR1 | TFPIE | GYTFTTF | GYTFTTFPIE | TTFPIE |
| | (SEQ ID NO: 61) | (SEQ ID NO: 73) | (SEQ ID NO: 74) | (SEQ ID NO: 75) |
| VH CDR2 | NFHPYNDDTKYNEKFKG | HPYNDD | NETIPYNDDTK | WMGNFHPYNDM |
| | (SEQ ID NO: 62) | (SEQ ID NO: 76) | (SEQ ID NO: 77) | (SEQ ID NO: 78) |
| VH CDR3 | RGKLPFDS | RGKLPFDS | RGKLPFDS | ARRGKLPFD |
| | (SEQ ID NO: 63) | (SEQ ID NO: 63) | (SEQ ID NO: 63) | (SEQ ID NO: 79) |
| VL CDR1 | RASQDISNYLN | RASQDISNYLN | RASQDISNYLN | SNYLNWY |
| | (SEQ ID NO: 64) | (SEQ ID NO: 64) | (SEQ ID NO: 64) | (SEQ ID NO: 80) |
| VL CDR2 | FTSRLRS | FTSRLRS | FTSRLRS | LLIYFTSRLR |
| | (SEQ ID NO: 65) | (SEQ ID NO: 65) | (SEQ ID NO: 65) | (SEQ ID NO: 81) |
| VL CDR3 | QQDRKLPWT | QQDRKLPWT | QQDRKLPWT | Q(JaKLPW |
| | (SEQ ID NO: 66) | (SEQ ID NO: 66) | (SEQ ID NO: 66) | (SEQ ID NO: 82) |

The anti-CD40 antibodies or antigen binding fragments thereof can encompass the heavy chain CDR 1, CDR2, and CDR3 and the light chain CDR 1, CDR2, and CDR3 of Exemplary Anti-CD40 Antibody 1. These antibodies can have, e.g., 1, 2, or 3 substitutions within one or more (i.e., 1, 2, 3, 4, 5, or 6) of the six CDRs of Exemplary Anti-CD40 Antibody 1. These antibodies (i) inhibit the humoral response to tetanus toxoid immunization in a primate without B cell depletion compared to vehicle; and/or (ii) do not elevate IL-12 serum levels compared to vehicle; and/or (iii) bind human or cynomolgus monkey CD40 with high affinity (e.g., K_{D}≦3 nM (monovalent affinity), K_{D}≦10 pM (bivalent affinity)) but do not significantly bind CD40 from rodents; and/or (iv) bind to an epitope within cysteine-rich domain 2 (CRD2) and cysteine-rich domain 3 (CRD3) of the extracellular domain of human and cynomolgus CD40; and/or (v) possess low effector activity compared to anti-CD40 antibodies G28.5 or ADH9; and/or (vi) have low agonistic activity in whole blood assays compared to anti-CD40 antibodies G28.5 or ADH9; and/or (vii) inhibit B cell activation by CD40L; and/or (viii) does not agonize platelets stimulated by soluble CD40L compared with the anti-CD40 antibody, G28.5; and/or (viii) have reduced binding as compared to a wild type IgG1 antibody to CD16a, CD32a, CD32b, and/or CD64; and/or (ix) bind to a protein encoded by a human CD40 DNA sequence containing any of the following human CD40 SNPs: A25S; S124L; I134V; F158L; S166R; S65R; D69E; H78Q; H80R; R90W; I134L; I134T; and V138F comparably as to wild type human CD40 (SEQ ID NO:58).

The anti-CD40 antibodies or antigen binding fragments thereof can comprise the heavy chain CDR 1 (VH-CDR1), CDR2 (VH-CDR2), and CDR3 (VH-CDR3) of Exemplary Anti-CD40 Antibody 1 according to the Kabat definition, or an alternate CDR definition such as, but not limited to, the Chothia from Abysis definition, the enhanced Chothia/AbM CDR definition, or the contact definition. These anti-CD40 antibodies may include zero, one, two, or three substitutions in VH-CDR1 and/or VH-CDR2 and/or VH-CDR3 of Exemplary Anti-CD40 Antibody 1. These antibodies (i) inhibit the humoral response to tetanus toxoid immunization in a primate without B cell depletion compared to vehicle; and/or (ii) do not elevate IL-12 serum levels compared to vehicle; and/or (iii) bind human or cynomolgus monkey CD40 with high affinity (e.g., KD≦3 nM (monovalent affinity), KD≦10 pM (bivalent affinity)) but do not significantly bind CD40 from rodents; and/or (ivi) bind to an epitope within cysteine-rich domain 2 (CRD2) and cysteine-rich domain 3 (CRD3) of the extracellular domain of human and cynomolgus CD40; and/or (viii) possess low effector activity compared to anti-CD40 antibodies G28.5 or ADH9; and/or (ivi) have low agonistic activity in whole blood assays compared to anti-CD40 antibodies G28.5 or ADH9; and/or (v) inhibit humoral response without B cell depletion; and/or (vii) inhibit B cell activation by CD40L; and/or (viii) does not agonize platelets stimulated by soluble CD40L compared with the anti-CD40 antibody, G28.5; and/or (viii) have reduced binding as compared to a wild type IgG1 antibody to CD16a, CD32a, CD32b, and/or CD64; and/or (ix) bind to a protein encoded by a human CD40 DNA sequence containing any of the following human CD40 SNPs: A25S; S124L; I134V; F158L; S166R; S65R; D69E; H78Q; H80R; R90W; I134L; I134T; and V138F comparably as to wild type human CD40. In some embodiments, the anti-CD40 antibodies further comprise the light chain CDR 1 (VL-CDR1), CDR2 (VL-CDR2), and CDR3 (VL-CDR3) of Exemplary Anti-CD40 Antibody 1 according to the Kabat definition, or an alternate CDR definition such as the Chothia from Abysis definition, the enhanced Chothia/AbM CDR definition, or the contact definition. These anti-CD40 antibodies may include zero, one, two, or three substitutions in VL-CDR1 and/or VL-CDR2 and/or VL-CDR3 of Exemplary Anti-CD40 Antibody 1.

In certain embodiments, the anti-CD40 antibodies or antigen binding fragments thereof comprise an amino acid sequence having at least 80%, 85%, 86%, 87%, 88%, 89%, 90%, 91%, 92%, 93%, 94%, 95%, 96%, 97%, 98%, 99%, or 100% identity to the variable heavy chain of Exemplary Anti-CD40 Antibody 1. In some embodiments, the anti-CD40 antibodies comprise an amino acid sequence having at least 80%, 85%, 86%, 87%, 88%, 89%, 90%, 91%, 92%, 93%, 94%, 95%, 96%, 97%, 98%, 99%, or 100% identity to the heavy chain of Exemplary Anti-CD40 Antibody 1. In certain embodiments, the anti-CD40 antibodies comprise an amino acid sequence having at least 80%, 85%, 86%, 87%, 88%, 89%, 90%, 91%, 92%, 93%, 94%, 95%, 96%, 97%, 98%, 99%, or 100% identity to the variable heavy chain and the variable light chain of Exemplary Anti-CD40 Antibody 1. In some embodiments, the anti-CD40 antibodies comprise an amino acid sequence having at least 80%, 85%, 86%, 87%, 88%, 89%, 90%, 91%, 92%, 93%, 94%, 95%, 96%, 97%, 98%, 99%, or 100% identity to the heavy chain and comprise an amino acid sequence having at least 80%, 85%, 86%, 87%, 88%, 89%, 90%, 91%, 92%, 93%, 94%, 95%, 96%, 97%, 98%, 99%, or 100% identity to the light chain of Exemplary Anti-CD40 Antibody 1. These antibodies (i) inhibit the humoral response to tetanus toxoid immunization in a primate without B cell depletion compared to vehicle; and/or (ii) do not elevate IL-12 serum levels compared to vehicle; and/or (iii) bind human or cynomolgus monkey CD40 with high affinity KD≦3 nM (monovalent affinity), KD≦10 pM (bivalent affinity)) but do not significantly bind CD40 from rodents; and/or (iv) bind to an epitope within cysteine-rich domain 2 (CRD2) and cysteine-rich domain 3 (CRD3) of the extracellular domain of human and cynomolgus CD40; and/or (v) possess low effector activity compared to anti-CD40 antibodies G28.5 or ADH9; and/or (vi) have low agonistic activity in whole blood assays compared to anti-CD40 antibodies G28.5 or ADH9; and/or (vii) inhibit B cell activation by CD40L; and/or (viii) does not agonize platelets stimulated by soluble CD40L compared with the anti-CD40 antibody, G28.5; and/or (ix) have reduced binding as compared to a wild type IgG1 antibody to CD16a, CD32a, CD32b, and/or CD64; and/or (x) bind to a protein encoded by a human CD40 DNA sequence containing at least one of the following human CD40 SNPs: A25S; S124L; I134V; F158L; S166R; S65R; D69E; H78Q; H80R; R90W; I134L; I134T; and V138F comparably as to wild type human CD40.

Exemplary Anti-CD40 Antibody 1 contacts amino acid residues 19 (Q), 21-22 (KY), 24-27 (DPNL) of human CD40 CRD2 (SEQ ID NO:51) and amino acid residues 9 (T) and 14-18 (ESCVL) of human CD40 CRD3 (SEQ ID NO:54). This disclosure features antibodies or antigen-binding fragments thereof that bind to the same epitope as Exemplary Anti-CD40 Antibody 1. This disclosure also features antibodies or antigen-binding fragments thereof that competitively inhibit binding of Exemplary Anti-CD40 Antibody 1 to human CD40.

In some embodiments, the variable heavy chain of Exemplary Anti-CD40 Antibody 1 is linked to a heavy chain constant region comprising a CH1 domain and a hinge region. In some embodiments, the variable heavy chain of Exemplary Anti-CD40 Antibody 1 is linked to a heavy chain constant region comprising a CH3 domain. In certain embodiments, the variable heavy chain of Exemplary Anti-CD40 Antibody 1 is linked to a heavy chain constant region comprising a CH1 domain, hinge region, and CH2 domain from IgG4 and a CH3 domain from IgG1. In certain embodiments such a chimeric antibody contains one or more additional mutations in the heavy chain constant region that increase the stability of the chimeric antibody. In certain embodiments, the heavy chain constant region includes substitutions that modify the properties of the antibody (e.g., decrease Fc receptor binding, increase or decrease antibody glycosylation, decrease binding to C1q).

In certain embodiments, the anti-CD40 antibody is an IgG antibody. In one embodiment, the antibody is IgG4. In another embodiment, the antibody is IgG2. In some embodiments, the antibody has a chimeric heavy chain constant region (e.g., having the CH1, hinge, and CH2 regions of IgG4 and CH3 region of IgG1). In certain embodiments, the antibody includes a human Fc region that binds human CD16a, human CD32a, human CD32b, and human CD64 with a reduced binding affinity as compared to a wild type IgG1 antibody (e.g., chimeric AKH3 IgG1). The Table below provides a list of some of the properties of Exemplary Anti-CD40 Antibody 1.

| | |
|---|---|
| Molecular Mass (calculated/actual) | Intact mAb: 144999.0 Da/145007 Da |
| | Heavy Chain: 48835.1Da/48830 Da |
| | Light Chain: 23680.5 Da/23675 Da |
| Molecular Mass (SDS-PAGE) | 150,000 Da |
| Extinction Coefficient (mg/mL) | 1.42 |
| Absorbance Maximum | 275 nm |
| pI (calculated) | 8.69 |
| pI (IEF) | Major component: 9.03 (55.2%) |
| | Acidic components: 8.94(41.4%; |
| | pI range 8.40 to 8.96) |
| | Basic component: 9.08 (3.4%) |
| Dissociation constant by BIAcore: | |
| human CD40 | 3 nM |
| cynomolgus monkey CD40 | 3 nM |
| Tm by DSC: | CH2: 66.2 ± 0.2° C. |
| | Fab: 80.9 ± 0.6° C., 83.5 ± 0.2° C. |
| | CH3: 72.8 ± 0.5° C. |
| Disulfide structure | Major as predicted; 2.8% mis-linkage |
| | Cys131/Cys144 |
| Free SH | 0.41/mole (1.3%) |
| Glycation | 0.25 mole/mole of Exemplary |
| | Anti-CD40 Antibody 1 |
| N-linked glycosylation | G0 (87%) |
| | G1 (4.6%) |
| | G2 (0.4%) |
| | Afucosyl (0.5%) |
| | Aglycosyl (0.5%) |
| Solubility in formulation buffer | >200 mg/mL |
| Aggregation (SEC) | 0.64% |
| T_{1/2} | 6-8 days in cynomolgus monkeys |
| | at 10 and 30 mg/kg |

Exemplary Anti-CD40 Antibody 1 exhibits suitable physicochemical properties for an antibody therapeutic. This antibody shows low levels of aggregation and can be formulated at concentrations that permit subcutaneous administration. The antibody can be formulated, e.g., at 50 mg/mL in a buffer (e.g., citrate or histidine buffer at pH 6.0). This antibody can also be formulated in a buffer (e.g., citrate or histidine buffer at pH 6.0) at much higher concentrations, such as 100-200 mg/mL (e.g., 100 mg/mL, 125 mg/mL, 150 mg/mL, 175 mg/mL, 200 mg/mL) or 150-300 mg/mL (e.g., 150 mg/mL, 175 mg/mL, 200 mg/mL, 225 mg/mL, 250 mg/mL, 275 mg/mL, 300 mg/mL).

Antibodies, such as Exemplary Anti-CD40 Antibody 1 or antigen binding fragments thereof, can be made, for example, by preparing and expressing nucleic acids that encode the amino acid sequences of the antibody. Moreover, this antibody and other anti-CD40 antibodies can be obtained, e.g., using one or more of the following methods.

In one embodiment, variant forms of anti-CD40 antibodies can be made, e.g., that vary in their glycosylation profile from that described above. For example, in certain embodiments it is desirable to produce an anti-CD40 antibody or antibody preparation that comprises reduced afucose content or increased fucose content. Anti-CD40 antibodies of the present invention with reduced afucose content (e.g., 0.1% to 1.5% afucose) have reduced whole blood agonism compared with anti-CD40 antibodies with increased afucose content (e.g., >5% afucose content). Whole blood agonism is measured, for example, by incubating whole blood overnight with anti-CD40 antibody in the presence of IL-4 and measuring upregulation of the activation marker, CD69, on B cells. In certain embodiments, the anti-CD40 antibody has 0.1% to 1.5% afucosyl content (e.g., 0.1%, 0.25%, 0.5%, 0.625%, 1%, 1.25%, 1.5%). In other embodiments, the anti-CD40 antibody has 0.1% to 1.0% afucosyl content. In other embodiments, the anti-CD40 antibody has 0.1% to 0.9% afucosyl content. In other embodiments, the anti-CD40 antibody has 0.1% to 0.8% afucosyl content. In other embodiments, the anti-CD40 antibody has 0.1% to 0.7% afucosyl content. In other embodiments, the anti-CD40 antibody has 0.1% to 0.6% afucosyl content. In other embodiments, the anti-CD40 antibody has 0.1% to 0.5% afucosyl content. In other embodiments, the anti-CD40 antibody has 0.1% to 0.4% afucosyl content. In other embodiments, the anti-CD40 antibody has 0.1% to 0.3% afucosyl content. In other embodiments, the anti-CD40 antibody has 0.1% to 0.2% afucosyl content.

In another embodiment, variant forms of anti-CD40 antibodies can be made that vary in their galactose and/or mannose profile. Antibodies with reduced galactose content and/or reduced high mannose can be made in CHO cells. In certain embodiments the G0 glycan content of the variant anti-CD40 antibody is approximately 1.5, L7, 1.8, 2, 2.2, 2.5, 3, 3.5, or 4-fold higher than the level of G0 glycan present in Exemplary anti-CD40 Antibody 1. In certain embodiments, the anti-CD40 antibodies have reduced galactose and/or reduced high mannose content. Levels of high-mannose glycans in the variant forms of the anti-CD40 antibodies can range from about 1% to about 25%, whereas endogenous human IgG contains only trace levels (<0.1%) of high-mannose glycans. Methods of altering high mannose content are well known in the art (see, e.g., Pacis et al., Biotechnol Bioeng., Volume 108, Issue 10, pages 2348-2358, October 2011; Shanta Raju, BioProcess Technical, April 2003; WO2013114245 A1, all incorporated by reference in their entireties). In certain embodiments, the anti-CD40 antibodies are produced a in culture medium comprising divalent manganese ion or its salts at a pH of about 6.8 to about 7.2.

In certain embodiments, variant forms of anti-CD40 antibodies can be made that vary in their galactose and/or mannose profile (e.g., reduced high mannose and/or reduced galactose content) as well as having reduced afucose content or increased fucose content. Other exemplary modifications to glycosylation and other parameters are set forth in more detail below.

### Methods of Obtaining Anti-CD40 Antibodies

Numerous methods are available for obtaining antibodies, particularly human antibodies. One exemplary method includes screening protein expression libraries, e.g., phage or ribosome display libraries. Phage display is described, for example, in U.S. Pat. No. 5,223,409; Smith, Science 228:1315-1317 (1985); WO 92/18619; WO 91/17271; WO 92/20791; WO 92/15679; WO 93/01288; WO 92/01047; WO 92/09690; and WO 90/02809. The display of Fab's on phage is described, e.g., in U.S. Pat. Nos. 5,658,727; 5,667,988; and 5,885,793.

In addition to the use of display libraries, other methods can be used to obtain a CD40-binding antibody. For example, the human CD40 protein or a peptide thereof can be used as an antigen in a non-human animal, e.g., a rodent, e.g., a mouse, hamster, or rat. In addition, cells transfected with a cDNA encoding human CD40 can be injected into a non-human animal as a means of producing antibodies that effectively bind the cell surface human CD40 protein.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al., Nature Genetics 7:13-21 (1994), U.S. 2003-0070185, WO 96/34096, and WO 96/33735. Such methods allow the preparation of fully human anti-CD40 antibodies.

In another embodiment, a monoclonal antibody is obtained from the non-human animal, and then modified, e.g., humanized or deimmunized. Winter describes an exemplary CDR-grafting method that may be used to prepare humanized antibodies described herein (U.S. Pat. No. 5,225,539). All or some of the CDRs of a particular human antibody may be replaced with at least a portion of a non-human antibody. It may only be necessary to replace the CDRs required for binding or binding determinants of such CDRs to arrive at a useful humanized antibody that binds to human CD40.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

A non-human CD40-binding antibody may also be modified by specific deletion of human T cell epitopes or “deimmunization” by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region, e.g., human IgG1 or kappa constant regions.

In some cases, a potential T cell epitope will include residues known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs can be eliminated by substitutions outside the CDRs. In some cases, an alteration within a CDR is the only option, and thus variants with and without this substitution can be tested. In other cases, the substitution required to remove a potential T cell epitope is at a residue position within the framework that might be critical for antibody binding. In these cases, variants with and without this substitution are tested. Thus, in some cases several variant deimmunized heavy and light chain variable regions are designed and various heavy/light chain combinations are tested to identify the optimal deimmunized antibody. The choice of the final deimmunized antibody can then be made by considering the binding affinity of the different variants in conjunction with the extent of deimmunization, particularly, the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005/0037000.

### Affinity Maturation

In one embodiment, an anti-CD40 antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad USA, 98:75-80 (2001); and in silico approaches (e.g., Lippow et al., Nat. Biotechnol., 25:1171-6 (2005).

In some implementations, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In one embodiment, an antibody has CDR sequences (e.g., a Chothia or Kabat CDR) that differ from those of the Exemplary Anti-CD40 Antibody 1. CDR sequences that differ from those of the Exemplary Anti-CD40 Antibody 1 include amino acid changes, such as substitutions of 1, 2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, praline, phenylalanine, methionine, tryptophan), beta-branched side chains (e.g., threonine, valine, isoleucine), and aromatic side chains (e.g., tyrosine, phenylalanine, tryptophan, histidine). In other embodiments, the amino acid substitution(s) is a non-conservative substitution. Such substitutions are within the ordinary skill of an artisan. The antibody or antibody fragments thereof that contain the substituted CDRs can be screened to identify antibodies having one or more of the features described herein (e.g., competing for binding to the extracellular domain of CD40 with Exemplary Anti-CD40 Antibody 1; binding the same or overlapping epitope as Exemplary anti-CD40 Antibody 1; selectively binding the extracellular domain of human and cynomolgus CD40, but not binding rodent CD40 or binding to rodent CD40 with a lower binding affinity than to human, cynomolgus, or rhesus CD40; exhibiting reduced agonistic activity in whole blood cultures compared to other anti-CD40 antibodies while maintaining formation of desired antibody dimers; inhibiting the humoral response to tetanus toxoid immunization in a primate without B cell depletion compared to vehicle; not elevating IL-12 serum levels in a primate compared to vehicle; binding to a CD40 protein encoded by a DNA molecule that contains the human CD40 SNP C77F about 50% as well as to wild type CD40 protein (SEQ ID NO:58); and/or binding to a protein encoded by a DNA molecule that contains the human CD40 H78Q comparably as to wild type CD40 protein (SEQ ID NO:58)).

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J. Immun., 147:2657-62 (1991); Morgan et al., Immunology, 86:319-24 (1995)), or changing the species from which the constant region is derived.

The anti-CD40 antibodies can be in the form of full length antibodies, or in the form of low molecular weight forms (e.g., biologically active antibody fragments or minibodies) of the anti-CD40 antibodies, e.g., Fab, Fab′, F(ab′)2, Fv, Fd, dAb, scFv, and sc(Fv)2. Other anti-CD40 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

The anti-CD40 antibodies can also be in the form of a monovalent antibody fragment comprising a single target molecule (e.g., human CD40) binding arm and an Fc region (i.e., a complex of Fc polypeptides). Such monovalent antibody fragments are generally more stable in vivo than a counterpart monovalent antibody fragment lacking the Fc region. In certain embodiments, the single human CD40 binding arm is an scFv. In other embodiments, the single human CD40 binding arm comprises two polypeptides. For example, the monovalent antibody fragment comprises: (i) a first polypeptide comprising a light chain variable domain (and in some embodiments further comprising a light chain constant domain, (CL)), (ii) a second polypeptide comprising a heavy chain variable domain, a first Fc polypeptide sequence (and in some embodiments further comprising a non-Fc heavy chain constant domain sequence), and (iii) a third polypeptide comprising a second Fc polypeptide sequence. Generally, the second polypeptide is a single polypeptide comprising a heavy chain variable domain, heavy chain constant domain (e.g., all or part of CH1) and the first Fc polypeptide. For example, the first Fc polypeptide sequence is generally linked to the heavy chain constant domain by a peptide bond (i.e., not a non-peptidyl bond). In one embodiment, the first polypeptide comprises a light chain variable domain described herein fused to a human light chain constant domain. In one embodiment, the second polypeptide comprises a human heavy chain variable domain described herein fused to a human heavy chain constant domain. In one embodiment, the third polypeptide comprises an N-terminally truncated heavy chain which comprises at least a portion of a hinge sequence at its N terminus. In one embodiment, the third polypeptide comprises an N-terminally truncated heavy chain which does not comprise a functional or wild type hinge sequence at its N terminus. In some embodiments, the two Fc polypeptides of an antibody fragment of the invention are covalently linked. For example, the two Fc polypeptides may be linked through intermolecular disulfide bonds, for instance through intermolecular disulfide bonds between cysteine residues of the hinge region. In some embodiments, the two Fc polypeptides of the monovalent antibody fragment are linked through a peptide linker (e.g., (SEQ ID NO:68)ₙ where n is 0, 1, 2, 3, 4, 5, 6, 7, 8, 9, or 10). In certain embodiments, “knobs into holes” mutations (see, e.g., Merchant et al., Nature Biotechnol., 16:677-681 (1998)) are present in the CH3 domains of the Fc polypeptides of the monovalent antibody fragment. For example, the “hole” mutations (T366S, L368A, Y407V) are made in the first Fc polypeptide and the “knob” mutation (T366W) is made in the second Fc polypeptide, or vice versa. In a specific embodiment, the monovalent antibody fragment comprises a single human CD40 binding arm (i.e., a first polypeptide comprising a VL-CL polypeptide, a second polypeptide comprising a VH-CH1-hinge-CH2-CH3 polypeptide), and a third polypeptide that comprises the Fc fragment (and optionally part or all of the hinge) of a heavy chain but does not comprise the VH or CH1 domains. In another specific embodiment, the monovalent antibody fragment comprises a single human CD40 binding arm (i.e., a first polypeptide comprising a scFv comprising a VH and VL region of a CD40 antibody described herein conjugated (directly or via a peptide linker) to a hinge-CH2-CH3 region of an Fc polypeptide), and a second polypeptide that comprises the Fc fragment (and optionally part or all of the hinge) of a heavy chain but does not comprise the VH or CH1 domains. Provided herein are compositions comprising a mixture of an anti-CD40 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-CD40 antibody or antigen-binding fragment thereof comprising at least one deamidation site, wherein the pH of the composition is from about 5.0 to about 6.5, such that, e.g., at least about 90% of the anti-CD40 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-CD40 antibody or antigen-binding fragment thereof, means the presence of both the desired anti-CD40 antibody or antigen-binding fragment thereof and one or more acidic variants thereof. The acidic variants may comprise predominantly deamidated anti-CD40 antibody, with minor amounts of other acidic variant(s).

In certain embodiments, the binding affinity (K_{D}), on-rate (K_{D} on) and/or off-rate (K_{D} off) of the anti-CD40 antibody that was mutated to eliminate deamidation is similar to that of the anti-CD40 wild-type antibody, e.g., having a difference of less than about 5 fold, 4 fold, 3 fold, 2 fold, or 1 fold.

### Antibody Fragments

Antibody fragments (e.g., Fab, Fab′, F(ab′)2, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Minibodies of anti-CD40 antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A., 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-CD40 antibody or antigen-binding fragment thereof described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order. Examples of arrangements are listed below:

[VL] linker [VH] linker [VH] linker [VL]

[VH] linker [VL] linker [VL] linker [VH]

[VH] linker [VH] linker [VL] linker [VL]

[VL] linker [VL] linker [VH] linker [VH]

[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18) can be used as a linker. Examples of such peptide linkers include: Ser; Gly Ser; Gly Gly Ser; Ser Gly Gly; Gly Gly Gly Ser (SEQ ID NO:60); Ser Gly Gly Gly (SEQ ID NO:67); Gly Gly Gly Gly Ser (SEQ ID NO:68); Ser Gly Gly Gly Gly (SEQ ID NO:69); Gly Gly Gly Gly Gly Ser (SEQ ID NO:70); Ser Gly Gly Gly Gly Gly (SEQ ID NO: 71); Gly Gly Gly Gly Gly Gly Ser (SEQ ID NO: 72); Ser Gly Gly Gly Gly Gly Gly (SEQ ID NO:86); (Gly Gly Gly Gly Ser (SEQ ID NO: 68)ₙ, wherein n is an integer of one or more; and (Ser Gly Gly Gly Gly (SEQ ID NO:69)ₙ, wherein n is an integer of one or more.

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the minibodies may include modifications such as substitutions, deletions, additions, and/or insertions. For example, the modification may be in one or more of the CDRs of the anti-CD40 antibody or antigen-binding fragment thereof (e.g., Exemplary Anti-CD40 Antibody 1). In certain embodiments, the modification involves one, two, or three amino acid substitutions in one or more CDRs of the VH and/or VL domain of the anti-CD40 minibody. Such substitutions are made to improve the binding and/or functional activity of the anti-CD40 minibody. In other embodiments, one, two, or three amino acids of the CDRs of the anti-CD40 antibody or antigen-binding fragment thereof may be deleted or added as long as there is CD40 binding and/or functional activity when VH and VL are associated.

### Bispecific Antibodies

Bispecific antibodies are antibodies that have binding specificities for at least two different epitopes. Exemplary bispecific antibodies may bind to two different epitopes of the CD40 protein. Other such antibodies may combine a CD40 binding site with a binding site for another protein (e.g., B7.1 (CD80), B7.2 (CD86), and LT-β receptor). Bispecific antibodies can be prepared as full length antibodies or low molecular weight forms thereof (e.g., RA′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The anti-CD40 antibodies described herein can be multivalent antibodies with three or more antigen binding sites (e.g., tetravalent antibodies), which can be readily produced by recombinant expression of nucleic acid encoding the polypeptide chains of the antibody. The anti-CD40 multivalent antibody can comprise a dimerization domain and three or more antigen binding sites. An exemplary dimerization domain comprises (or consists of) an Fc region or a hinge region. An anti-CD40 multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I) fluorescent substances, luminescent substances, haptens, enzymes, metal chelates, and drugs.

In certain embodiments, an anti-CD40 antibody or antigen-binding fragment thereof are modified with a moiety that improves its stabilization and/or retention in circulation, e.g., in blood, serum, or other tissues, e.g., by at least 1.5, 2, 5, 10, 15, 20, 25, 30, 40, or 50 fold. For example, the anti-CD40 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CD40 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. No. 5,057,313 and U.S. Pat. No. 5,156,840).

Antibodies with Reduced Effector Function

The interaction of antibodies and antibody-antigen complexes with cells of the immune system triggers a variety of responses, referred to herein as effector functions. Immune-mediated effector functions include two major mechanisms: antibody-dependent cell-mediated cytotoxicity (ADCC) and complement-dependent cytotoxicity (CDC). Both of them are mediated by the constant region of the immunoglobulin protein. The antibody Fc domain is, therefore, the portion that defines interactions with immune effector mechanisms.

IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. These responses can provoke unwanted side effects such as inflammation and the elimination of antigen-bearing cells. Accordingly, the present invention further relates to CD40-binding proteins, including antibodies, with reduced effector functions.

Effector function of an anti-CD40 antibody of the present invention may be determined using one of many known assays. The anti-CD40 antibody's effector function may be reduced relative to a second anti-CD40 antibody. In some embodiments, the second anti-CD40 antibody may be any antibody that binds CD40 specifically. In other embodiments, the second CD40-specific antibody may be any of the antibodies of the invention, such as chimeric AKH3-IgG1 (see, Example 2) or Exemplary Anti-CD40 Antibody 1. In other embodiments, where the anti-CD40 antibody of interest has been modified to reduce effector function, the second anti-CD40 antibody may be the unmodified or parental version of the antibody.

Effector functions include ADCC, whereby antibodies bind Fc receptors on cytotoxic T cells, natural killer (NK) cells, or macrophages leading to cell death, and CDC, which is cell death induced via activation of the complement cascade (reviewed in Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative is to use the IgG4 subtype antibody, which binds to FcγRI but which binds poorly to C1q and FcγRII and RIII. However, IgG4 antibodies may form aggregates since they have poor stability at low pH compared with IgG1 antibodies. The stability of an IgG4 antibody can be improved by substituting arginine at position 409 (according to the EU index proposed by Kabat et al., Sequences of proteins of immunological interest, 1991, 5^{th}) with any one of: lysine, methionine, threonine, leucine, valine, glutamic acid, asparagine, phenylalanine, tryptophan, or tyrosine. Alternatively, and or in addition, the stability of an IgG4 antibody can be improved by substituting a CH3 domain of an IgG4 antibody with a CH3 domain of an IgG1 antibody, or by substituting the CH2 and CH3 domains of IgG4 with the CH2 and CH3 domains of IgG1. Accordingly, the anti-CD40 antibodies of the present invention that are of IgG4 isotype can include modifications at position 409 and/or replacement of the CH2 and/or CH3 domains with the IgG1 domains so as to increase stability of the antibody while decreasing effector function. The IgG2 subtype also has reduced binding to Fc receptors, but retains significant binding to the H131 allotype of FcγRIIa and to C1q. Thus, additional changes in the Fc sequence may be required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Both FcγRII and FcγRIII have two types: FcγRIIa (CD32a) and FcγRIIB (CD32b); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues G233-S239 (EU index numbering as in Kabat et al., Sequences of Proteins of Immunological Interest, 5th Ed. Public Health Service, National Institutes of Health, Bethesda, Md. (1991), Woof et al., Molec. Immunol. 23:319-330 (1986); Duncan et al., Nature 332:563 (1988); Canfield and Morrison, J. Exp. Med. 173:1483-1491 (1991); Chappel et al., Proc. Natl. Acad. Sci USA 88:9036-9040 (1991)). Of residues G233-S239, P238 and S239 are among those cited as possibly being involved in binding. Other residues involved in binding to FcγR are: G316-K338 (Woof et al., Mol. Immunol., 23:319-330 (1986)); K274-R301 (Sammy et al., Molec. Immunol. 21:43-51 (1984)); Y407-R416 (Gergely et al., Biochem. Soc. Trans. 12:739-743 (1984) and Shields et al., J Biol Chem 276: 6591-6604 (2001), Lazar G A et al., Proc Natl Acad Sci 103: 4005-4010 (2006)); N297; T299; E318; L234-S239; N265-E269; N297-T299; and A327-I332. These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-CD40 antibodies of the present invention include modifications of one or more of the aforementioned residues to decrease effector function as needed.

Another approach for altering monoclonal antibody effector function include mutating amino acids on the surface of the monoclonal antibody that are involved in effector binding interactions (Lund, J., et al. (1991) J. Immunol. 147(8): 2657-62; Shields, R. L. et al. (2001) J. Biol. Chem. 276(9): 6591-604).

To reduce effector function, one can use combinations of different subtype sequence segments (e.g., IgG2 and IgG4 combinations) to give a greater reduction in binding to Fcγ receptors than either subtype alone (Armour et al., Eur. J. Immunol., 29:2613-1624 (1999); Mol. Immunol., 40:585-593 (2003)). A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260.

Anti-CD40 antibodies of the present invention with reduced effector function include antibodies with reduced binding affinity for one or more Fc receptors (FcRs) relative to a parent or non-variant anti-CD40 antibody. Accordingly, anti-CD40 antibodies with reduced FcR binding affinity includes anti-CD40 antibodies that exhibit a 1.5-fold, 2-fold, 2.5-fold, 3-fold, 4-fold, 5-fold, 10-fold, 20-fold, or 25-fold or higher decrease in binding affinity to one or more Fc receptors compared to a parent or non-variant anti-CD40 antibody. In some embodiments, an anti-CD40 antibody with reduced effector function binds to an FcR with about 10-fold less affinity relative to a parent or non-variant antibody. In other embodiments, an anti-CD40 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-CD40 antibody with reduced effector function exhibits a 1.5-fold, 2-fold, 2.5-fold, 3-fold, 4-fold, or 5-fold or higher decrease in binding affinity to FcγRIIa.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus, the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods, 202:163 (1996), may be performed.

It has been proposed that various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. No. 5,648,260, and U.S. Pat. No. 5,624,821).

Anti-CD40 antibodies with reduced C1q binding can comprise an amino acid substitution at one, two, three, or four of amino acid positions 270, 322, 329 and 331 of the human IgG Fc region, where the numbering of the residues in the IgG Fc region is that of the EU index as in Kabat. As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Accordingly, in certain embodiments, an anti-CD40 antibody of the present invention exhibits reduced binding to a complement protein relative to a second anti-CD40 antibody (e.g., chimeric AKH3-IgG1). In certain embodiments, an anti-CD40 antibody of the invention exhibits reduced binding to C1q by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-CD40 antibody (e.g., chimeric AKH3-IgG1).

Thus, in certain embodiments of the invention, one or more of these residues may be modified, substituted, or removed or one or more amino acid residues may be inserted so as to decrease CDC activity of the anti-CD40 antibodies provided herein.

In certain other embodiments, the present invention provides an anti-CD40 antibody that exhibits reduced binding to one or more FcR receptors but that maintains its ability to bind complement (e.g., to a similar or, in some embodiments, to a lesser extent than a native, non-variant, or parent anti-CD40 antibody). Accordingly, an anti-CD40 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-CD40 antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-CD40 antibody may be modulated by altering properties of the constant region, and the Fc region in particular. In certain embodiments, the anti-CD40 antibody having decreased effector function is compared with a second antibody with effector function and which may be a non-variant, native, or parent antibody comprising a native constant or Fc region that mediates effector function.

A native constant region comprises an amino acid sequence identical to the amino acid sequence of a constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. The variant constant region can have decreased effector function.

Antibodies with decreased effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns, may be achieved by manipulating the host cell and cell culture and expression conditions by which the antibody is produced.

Certain embodiments of the present invention relate to an anti-CD40 antibody comprising or consisting of one or more (1, 2, or 3) heavy chain CDR sequences (Kabat or alternate CDR) from SEQ ID NO:33. In one embodiment, the anti-CD40 antibody heavy chain CDR sequences comprise or consist of the amino acid sequences in SEQ ID NO:61, SEQ ID NO:62, and SEQ ID NO:63. These antibodies may also comprise or consist of one or more (1, 2, or 3) light chain CDR sequences (Kabat or alternate CDR) from SEQ ID NO: 34. For example, the anti-CD40 antibody light chain CDR sequences may comprise or consist of the amino acid sequences in SEQ ID NO:64, SEQ ID NO:65, and SEQ ID NO:66. The antibodies described herein may further comprise a Fc region (e.g., the Fc region of IgG4) that confers reduced effector function compared to a native or parental Fc region. These anti-CD40 antibodies (i) bind an epitope within CRD2 and CRD3 of human and cynomolgus CD40; and/or (ii) bind with high affinity (e.g., a KD≦3 nM) to human and cynomolgus CD40; and/or (iii) have low agonistic activity in whole blood assays; and/or (iv) inhibit humoral response without B cell depletion; and/or (v) inhibiting B cell activation by CD40L; and/or (vi) not agonizing platelets stimulated by soluble CD40L; and/or (vii) have reduced binding as compared to a wild type IgG1 antibody to CD16a, CD32a, CD32b, and/or CD64; and/or (viii) binding to a protein encoded by a CD40 DNA sequence that has any of the following human CD40 SNPs: A25S; S124L; I134V; F158L; S166R; S65R; D69E; H78Q; H80R; R90W; I134L; I134T; and V138F comparably as to wild type human CD40.

In other embodiments, the disclosure provides an anti-CD40 antibody comprising a VL sequence comprising SEQ ID NO:34 and a VH sequence comprising SEQ ID NO:33, the antibody further comprising an Fc region (e.g., IgG4 Fc region) or a variant Fc region that confers reduced effector function compared to a native or parental Fc region.

Methods of generating any of the aforementioned anti-CD40 antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

Anti-CD40 Antibodies with Altered Glycosylation

Different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the micro heterogeneity of human IgG oligosaccharides can affect biological functions such as CDC and ADCC, binding to various Fc receptors, and binding to C1q protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remains unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

The anti-CD40 antibodies of the present invention may be modified or altered to elicit decreased effector function(s) compared to a second CD40-specific antibody. Methods for altering glycosylation sites of antibodies are described, e.g., in U.S. Pat. No. 6,350,861 and U.S. Pat. No. 5,714,350, WO 05/18572 and WO 05/03175; these methods can be used to produce anti-CD40 antibodies of the present invention with altered, reduced, or no glycosylation.

Alternatively, the anti-CD40 antibodies of the present invention may be produced in a cell line which provides a desired glycosylation profile. For example, cells that make little afucosylated antibody, such as CHO cells, may be used for production.

In another embodiment, manufacturing processes and/or media content or conditions may be manipulated to modulate the galactose and/or high mannose content. In one embodiment, the galactose/high mannose content of the anti-CD40 antibody is low or reduced.

### Methods of Producing Antibodies

Antibodies or antigen binding fragments thereof may be produced in bacterial or eukaryotic cells. Some antibodies, e.g., Fab's, can be produced in bacterial cells, e.g., E. coli cells. Antibodies or antigen binding fragments thereof can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. In one embodiment, the anti-CD40 antibodies described herein are produced in the dihydrofolate reductase-deficient Chinese hamster ovary (CHO) cell line, DG44. In another embodiment, the anti-CD40 antibodies described herein are produced in the DG44i cell line. To produce the antibody or antigen binding fragments thereof of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, recombinant expression vectors encoding the antibody heavy chain and the antibody light chain of an anti-CD40 antibody, respectively (e.g., Exemplary Anti-CD40 Antibody 1) are introduced into dhfr⁻ CHO cells by calcium phosphate-mediated transfection. In a specific embodiment, the dhfr− CHO cells are cells of the DG44 cell line, such as DG44i (see, e.g., Derouaz et al., Biochem Biophys Res Commun. 340(4):1069-77 (2006)). Within the recombinant expression vectors, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The CD40-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-CD40 antibody) and a target (e.g., CD40) can be analyzed using the OCTET® systems. In this method, one of several variations of instruments (e.g., OCTET® QK^{e} and QK), made by the FortéBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-CD40 antibody) and a target (e.g., CD40) can be analyzed using Surface Plasmon Resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human CD40 using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

The function and/or activities of the anti-CD40 antibodies described herein (e.g., Exemplary anti-CD40 Antibody 1) can be compared with other reference or comparator antibodies. Non-limiting examples of such antibodies include the anti-CD40 monoclonal antibody, clone G28.5 (AbNova, Catalog Number MAB8023; BioLegend, Catalog No. 303602; Bishop, J. Immunol., 188(9):4127-29 (2012)), and ADH9. The Genbank accession number for the heavy chain variable region of G28.5 is AF013577 and for the light chain variable region is AF013576. The amino acid sequences of the heavy and light chains of the ADH9 antibody (human IgG1) are provided below (the signal peptide is boldened):

Light Chain:

(SEQ ID NO: 83)
MKLPVRLLVL MFWIPASSS DVVNITQTPLSL PVSLGDQASI
SCRSSQSLVH SNGNTYLHWY LQKPGQSPKL LIYKVSNRFS
GVPDRFSGSG SGTDFTLKIS RVEAEDLGVY FCSQSTEVPW
TFGGGTKLEI KRTVAAPSVF IFPPSDEQLK SGTASVVCLL
NNFYPREAKV QWKVDNALQS GNSQESVTEQ DSKDSTYSLS
STLTLSKADY EKHKVYACEV THQGLSSPVT KSFNRCEC

Heavy Chain:

(SEQ ID NO: 84)
MAVLGLLFCL VAFPSCVLS QVQLKESGPGL VAPSQSLSIT
CIVSGFSLTN SSVHWVRQPP GKGLEWLGII WAGGSTNYNS
ALMSRLSISK DNSKSQVFLK MNSLQTDDTA MYYCARVGGD
YWGQGTTLTV SSASTKGPSV FPLAPSSKST SGGIAALGCL
VKDYFPEPVT VSWNSGALTS GVHTFPAVLQ SSGLYSLSSV
VIVPSSSLGT QTYICNVNHK PSNTKVDKKV EPKSCDKTHT
CPPCPAPELL GGPSVFLFPP KPKDTLMISR TPEVTCVVVD
VSHEDPEVKF NWYVDGVEVH NAKTKPREEQ YNSTYRVVSV
LTVLHQDWLN GKEYKCKVSN KALPAPIEKT ISKAKGQPRE
PQVYTLPPSR DELTKNQVSL TCLVKGFYPS DIAVEWESNG
QPENNYKTTP PVLDSDGSFF LYSKLTVDKS RWQQGNVFSC
SVMHEALHNH YTQKSLSLSP G

The ADH9 human IgG4P heavy chain amino acid sequence is provided below (signal peptide boldened):

(SEQ ID NO: 85)
MAVLGLLFCL VAFPSCVLSQ VQLKESGPGL VAPSQSLSIT
CTVSGFSLTN SSVHWVRQPP GKGLEWLGII WAGGSTNYNS
ALMSPLSISK DNSKSQVFLK MNSLQTDDTA MYYCARVGGD
YWGQGTTLTV SSASTKGPSV FPLAPCSRST SESTAALGCL
VKDYFPEPVT VSWNSGALTS GVHTFPAVLQ SSGLYSLSSV
VTVPSSSLGT KTYTCNVDHK PSNTKVDKRV ESKYGPPCPP
CPAPEFLGGP SVFLFPPKPK DTLMISRTPE VTCVVVDVSQ
EDPEVQFNWY VDGVEVHNAK TKPREEQFNS TYPVVSVLTV
LHQDWLNGKE YKCKVSNKGL PSSIEKTISK AKGQPREPQV
YTLPPSQEEM TKNQVSLICL VKGFYPSDIA VEWESNGQPE
NNYKTTPPVL DSDGSFFLYS RLTVDKSRWQ EGNVFSCSVM
HEALHNHYTQ KSLSLSLG

The ADH9 human IgG4P/IgG1 heavy chain amino acid sequence is provided below (signal peptide boldened):

(SEQ ID NO: 57)
MAVLGLLFCL VAFPSCVLSQ VQLKESGPGL VAPSQSLSIT
CTVSGFSLTN SSVHWVRQPP GKGLEWLGII WAGGSTNYNS
ALMSRLSISK DNSKSQVFLK MNSLQTDDTA MYYCARVGGD
YWGQGTTLTV SSASTKGPSV FPLAPCSRST SESTAALGCL
VKDYFPEPVT VSWNSGALTS GVHTFPAVLQ SSGLYSLSSV
VTVPSSSLGT KTYTCNVDFIK PSNTKVDKRV ESKYGPPCPP
CPAPEFLGGP SVFLFPPKPK DTLMISRTPE VTCVVVDVSQ
EDPEVQFNWY VDGVEVHNAK TKPREEQFQS TYRVVSVLTV
LHQDWLNGKE YKCKVSNKGL PSSIEKTISK AKGQPREPQV
YTLPPSRDEL TKNQVSLTCL VKGFYPSDIA VEWESNGQPE
NNYKTTPPVL DSDGSFFLYS KLTVDKSRWQ QGNVFSCSVM
HEALHNHYTQ KSLSLSPG

### Indications

Anti-CD40 antibodies or antigen-binding fragments thereof described herein can be used to treat or prevent a variety of immunological disorders, such as autoimmune disorders, inflammatory diseases, disorders of humoral immunity, and fibrotic disorders. The anti-CD40 antibodies or antigen-binding fragments thereof of this disclosure are useful to treat or prevent such disorders at least because they inhibit or block the interaction of CD40 with its ligand, CD40L (CD154). CD40 signaling constitutes an important component in the activation of innate and adaptive immune functions, notably including B cell clonal expansion, differentiation to antibody forming cells (AFC) and memory cells expressing isotype-switched antibodies, the germinal center (GC) reaction, and optimal T helper effector cell responses.

CD40 signal transduction is induced upon engagement of CD40L which is rapidly but transiently expressed on the surface of CD4⁺ T cells following activation through the T cell receptor (TCR). In addition, platelets contain large amounts of CD40L that is translocated to their surface after activation. CD40L can be cleaved to release soluble ligand (sCD40L) and it is thought that platelets represent the largest source of circulating CD40L. The CD40L released by platelets may be particularly important in the activation of endothelial cells and leukocyte recruitment to sites of damage.

Engagement of CD40 on B cells delivers signals essential for B cell clonal expansion and their differentiation into plasma cells (PC) and memory cells producing class-switched antibodies. The critical role of CD40 in the generation of B cell memory is mediated by its nonredundant function in the generation and maintenance of GC, where antibody affinity is matured by somatic hypermutation (SHM), antigen-driven selection on FDC networks and signals from Tfh cells. Engagement of CD40 on dendritic cells (DC) and macrophages signals their priming, differentiation, and effector functions. CD40-stimulated B cells and other antigen-presenting cells (APC) may subsequently regulate T cells, providing for a positive feedback amplification mechanism for optimal T effector cell responses of the Th1, Th2, Th17 and Tfh types. Engagement of CD40 on these and many other cell types can lead to the production of inflammatory cytokines and chemokines, nitric oxide (NO) and matrix metalloproteinases (MMP). For example, interaction of CD40L with CD40⁺ endothelial cells results in the upregulation of critical adhesion molecules (V CAM-1, ICAM-1, and E-selectin) and leukocyte extravasation.

Hyperactivation of the CD40/CD40L pathway occurs in autoimmune and inflammatory diseases. Elevated levels of membrane or soluble CD40 or CD40L are seen in patients with autoimmune disorders, such as Sjögren's syndrome and systemic lupus erythematosus (SLE). Blockade of CD40L is efficacious in a wide range of models of inflammatory and autoimmune disease and humoral immunity in nonhuman primates and rodents, including its ability to reduce the generation and maintenance of titers of anti-coagulation factor VIII antibodies. Blockade of CD40 is also effective in modulating tissue injury and radiation induced lung injury. Therefore, blocking CD40 can potentially reduce all of these downstream effects of CD40 signaling, dampening the hyperactivation of adaptive and innate immune responses and downmodulating fibrotic disease in patients with autoimmune, inflammatory, and/or fibrotic disease.

This disclosure provides methods of blocking CD40 signaling using the anti-CD40 antibodies or antigen-binding fragments thereof of this disclosure. Such antibodies or antigen-binding fragments thereof are useful to in treating autoimmunity, inflammatory and/or fibrotic disease in patients. In addition, these antibodies are also useful in treating antibody-mediated diseases as well as neurological disorders.

The term “treating” refers to administering a composition comprising an anti-CD40 antibody or antigen-binding fragment thereof described herein in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

Autoimmune diseases that can be treated or prevented with anti-CD40 antibodies or antigen binding fragments thereof that are described herein include, e.g., Sjögren's syndrome (e.g. primary Sjögren's syndrome (pSS)), SLE (e.g., moderate or severe lupus), lupus nephritis, cutaneous lupus, discoid lupus, systemic sclerosis (scleroderma), acquired hemophilia, Crohn's disease, ulcerative colitis, Graves disease, Idiopathic thrombocytopenic purpura (ITP), rheumatoid arthritis (RA), asthma, vasculitis, pemphigoid, atopic dermatitis, and hemolytic anemia.

Antibody-mediated diseases or situations where the anti-CD40 antibodies or antigen binding fragments thereof that are described herein are useful include, e.g., hemophilia with inhibitors, transplant rejection, antibody cross-match pre-transplant, alloantibody in transfusion, and graft vs. host disease.

Neurological diseases that can be treated or prevented with anti-CD40 antibodies or antigen binding fragments thereof that are described herein include, e.g., myasthenia gravis, Alzheimer's disease, neuromyelitis optica (NMO), and Amyotrophic lateral sclerosis (ALS).

pSS is a systemic autoimmune disease, mainly involving the salivary and lacrimal glands with lymphocytic infiltration of these exocrine glands, leading to damage and loss of function. In addition to the salivary and lacrimal glands, other exocrine glands are also involved. It is the second-most prevalent autoimmune disease, with at least 1 million affected in the US. This disorder commonly affects middle-aged women, with a 9:1 female-to-male ratio and a peak incidence in their late 40s. This disorder is characterized by generalized dryness and typically symptoms of sicca. Patients with pSS generally experience sicca symptoms of xerostomia (dry mouth) and keratoconjunctivitis sicca (dry eyes). Patients are classified as having “primary” disease if they have sicca symptoms in the absence of another systemic disease such as SLE or RA, and providing they meet the classification criteria of pSS (Vitali et al., Ann. Rheum. Dis., 61:554-558 (2002)). These patients may also complain of losing their teeth and having difficulty of swallowing from decreased saliva secretion. In addition, the decrease in tear production will result in burning pain and sometimes severe local lesions (e.g., corneal abrasions/ulcers). Other exocrine glands may also be involved, causing recurrent parotiditis, a decrease in secretion of the upper and lower respiratory tract, and vaginal dryness. The histopathologic hallmark of pSS is a chronic mononuclear infiltrate of mostly T cells but also B cells and plasma cells in the exocrine glands. In addition to having sicca, profound fatigue, arthralgia/arthritis as well as Raynaud's phenomenon are relatively common extraglandular manifestations of pSS. Up to 30% of pSS patients will develop severe extraglandular complications involving the lung, kidney and/or neurological systems. Furthermore, 5% of pSS patients will eventually develop lymphoma (i.e., non-Hodgkin's lymphoma). Serologically, pSS subjects have signs of B cell hyperactivity and/or autoimmunity. A positive rheumatoid factor (RF) is present in about 50% of the cases. Also frequently observed is hypergammaglobulinemia or the presence of serum autoantibodies, including ANA (antinuclear autoantibody), anti-Sjögren's syndrome antigen A (SSA)/Ro and anti-Sjögren's syndrome antigen (SSB)/La antibodies. The revised criteria proposed by the American-European Consensus Group for pSS requires either a positive test for serum anti-SSA/SSB antibodies or the presence of focal lymphocytic sialadenitis as key criteria for diagnosis of pSS. In addition to a positive serology or a positive histopathology, subjects need to present with 3 of the following additional criteria: ocular symptoms of inadequate tear production; oral symptoms of decreased saliva production; ocular signs (Schirmer's test or positive rose Bengal score); or salivary gland involvement as evidenced by impaired salivary gland function (e.g., unstimulated whole salivary flow ≦1.5 mL/min or delayed uptake by sialoscintigraphy or sialectasias observed via parotid sialography). In addition to the above, the presence of any 3 of the 4 objective criteria (i.e., ocular signs, histopathology, salivary gland involvement, or anti-SSA/SSB antibodies) also qualifies the patient as having pSS. There are no disease modifying therapies approved for pSS and the management of the patients remains suboptimal. In particular, no biologics have been approved yet for the treatment of pSS. Thus, there is a clear unmet need to develop new therapies. CD40 and CD40L are expressed in the inflammatory foci in the salivary glands of pSS patients. CD40 is strongly expressed by the infiltrating lymphocytes, macrophages and DC, and also more weakly by epithelial cells. In addition, CD40 expression by cultured salivary gland epithelial cells is higher in those derived from pSS as compared to healthy subjects. CD40L is also elevated on the surface of activated CD4⁺ peripheral blood T cells and soluble CD40L levels are elevated in the serum of pSS patients. Given these obvious features of B cell hyperactivity in pSS and the principal role of CD40L/CD40 signaling for B cell activation and humoral immunity, blockade of CD40L binding to CD40 by the antibodies or antigen-binding fragments thereof described herein can reduce adaptive and innate immune cell activation in the following ways. First, the CD40 blockade will decrease B cell activation, clonal expansion, and terminal differentiation to autoantibody-forming cells. This is highly relevant in pSS where autoantibody production appears to derive from short-lived plasma cells rather than long-lived plasma cells. Second, the anti-CD40 antibodies or antigen-binding fragments thereof described herein have the potential to inhibit GC formation and the generation of autoreactive memory B cells in pSS patients. Notably, CD40L/CD40 signaling appears to be required for the maintenance of established GC and the anti-CD40 antibodies or antigen-binding fragments thereof described herein therefore have the potential to reduce the number and size of pre-existing GC-like structures in pSS glands which are associated with more severe clinical manifestations and risk of B cell lymphoma. Thus, the anti-CD40 antibodies or antigen-binding fragments thereof described herein may interfere with a vicious cycle in which the dysregulated production of autoantibodies promotes innate immune cell production of Type I IFN and other cytokines, which in turn further promotes dysregulated adaptive immunity. Finally, the anti-CD40 antibodies or antigen-binding fragments thereof described herein have the potential to reduce pathogenic Th effector responses in pSS by reducing CD40-mediated activation of B cells and other APC types that promote Th effector cell responses, including CD40-induced IL-12 production mediating the polarization of Th1 effector cells. By decreasing hyperactivity in both the innate and adaptive immune cell types in pSS patients, the anti-CD40 antibodies or antigen-binding fragments thereof described herein can reduce the systemic manifestations of the disease and improve the symptom of dryness. By decreasing B cell hyper-reactivity in the GC, the anti-CD40 antibodies or antigen-binding fragments thereof described herein can reduce the risk of B cell lymphoma.

The anti-CD40 antibodies or antigen-binding fragments thereof described herein are also useful in treating or preventing SLE in a patient in need thereof. SLE is a chronic autoimmune disease where multiple organs are damaged by immune complexes and tissue-binding autoantibodies (see, Guidelines for Referral and Management of Systemic Lupus Erythematosus in Adults, Arthritis & Rheumatism, 42(9):1785-1795 (1999)). Autoantibodies are present in SLE and may precede the development of the clinical disease (Arbuckle et al., N. Engl. J Med., 349(16):1526-33 (2003)). Internalization of the autoantibody containing immune complexes through Fc receptors leads to the production of type I interferon which in turn promotes loss of tolerance, perpetuating the vicious cycle of autoimmunity (Means et al., Ann N Y Acad Sci., 1062:242-51 (2005)). SLE is heterogeneous with regard to its clinical presentation, course, prognosis and genetics. African Americans share an increased risk for SLE that is often more severe as compared to white patients. Complement deficiencies were recognized early as risk factors for the development of SLE. More recently, genetic polymorphisms associated with type I interferon pathways have been described to confer susceptibility. For example, anti-double stranded DNA and anti-Ro auto-antibodies were associated with a certain haplotype of the transcription factor interferon regulatory factor 5 (IRF5). The haplotype also predicted high levels of IFN-α in the serum of SLE patients (Niewold et al., Ann. Rheum. Dis., 71(3):463-8 (2012)). Higher IFN-α levels have been correlated with a greater extent of multiple organ involvement in SLE patients (Bengtsson et al., Lupus, 9(9):664-71 (2000)). Furthermore, the so called “interferon signature” seems to be prominent in SLE. Interferon signature represents an mRNA expression pattern of interferon inducible genes. A type-I interferon signature is found in more than half of SLE patients and is associated with greater disease activity (Baechler et al., Proc. Natl. Acad. Sci USA, 100(5):2610-5 (2003)). IFN-α monoclonal antibodies have now entered the clinics and phase 1 results of sifalimumab and rontalizumab have demonstrated a dose-dependent reduction in type I IFN signature in the whole blood of SLE patients (McBride et al., Arthritis Rheum., 64(11):3666-76 (2012); Yao et al., Arthritis Rheum., (6):1785-96 (2009)). Validated indices have been developed for the assessment of disease activity and disease severity (e.g., moderate, severe) (see, e.g., Gladman, Prognosis and treatment of systemic lupus erythematosus, Curr. Opin. Rheumatol., 8:430-437 (1996); Kalunian et al., Definition, classification, activity and damage indices. In: Dubois' lupus erythematosus. 5^{th} ed., Baltimore: Williams and Wilkins; pp. 19-30 (1997)).

The anti-CD40 antibodies or antigen-binding fragments thereof described herein can also be used in treating or preventing scleroderma in a patient in need thereof. Systemic sclerosis or systemic scleroderma is a systemic autoimmune disease or systemic connective tissue disease that is a subtype of scleroderma. It is characterized by deposition of collagen in the skin and, less commonly, in the kidneys, heart, lungs and stomach. The female to male ratio for this disease is 4:1. The peak age of onset of the disease is between 30-50 years.

The anti-CD40 antibodies or antigen-binding fragments thereof described herein can also be used in treating or preventing hemophilia with inhibitors in a patient in need thereof. Approximately 15-20% of people with hemophilia will develop an antibody called an inhibitor to the product used to treat or prevent bleeding episodes. Developing an inhibitor is one of the most serious and costly complications of hemophilia. People with hemophilia use treatment products called factor clotting concentrates. This treatment improves blood clotting and is used to stop or prevent a bleeding episode. Inhibitors develop when the body's immune system stops accepting the factor (factor VIII for hemophilia A and factor IX for hemophilia B) as a normal part of blood. Thinking that the factor is a foreign substance, the body tries to destroy it using inhibitors. The inhibitors stop the factor from working. This makes it more difficult to stop a bleeding episode. People with hemophilia who develop an inhibitor do not respond as well to treatment. Inhibitors most often appear during the first year of treatment but they can appear at any time. A blood test is used to diagnose inhibitors. The blood test measures inhibitor levels (called inhibitor titers) in the blood. The amount of inhibitor titers is measured in Bethesda units (BU). The higher the number of Bethesda units, the more inhibitor is present. “Low titer” inhibitor has a very low measurement, usually less than or equal to 5 BU, whereas “high titer” inhibitor has a very high measurement, usually higher than 5 BU. Inhibitors are also labeled “low responding” or “high responding” based on how strongly a person's immune system reacts or responds to repeated exposure to factor concentrate. When people with high-responding inhibitors receive factor concentrates, the inhibitor titer measurement increases quickly. The increased inhibitor titer prevents the factor clotting concentrates from stopping or preventing a bleeding episode. Repeated exposure to factor clotting concentrates will cause more inhibitors to develop.

The anti-CD40 antibodies or antigen-binding fragments thereof described herein can also be used prophylactically in reducing antibody-mediated transplant rejection in a patient in need thereof. In transplantation, a positive cytotoxic crossmatch between donor cells and recipient serum is associated with early rejection or graft loss. A crossmatch is a test which determines if the recipient has antibodies to the potential donor. The crossmatch is performed by mixing a small amount of the patient's serum with a very small amount of the potential donor's white cells. If the patient has antibody to the donor's HLA, the donor's cells will be injured and this is referred to as a “positive crossmatch”. A positive crossmatch is a strong indication against transplant, since it signifies that the patient has the ability to attack the donor's cells, and would, most likely attack the donor's implanted organ/tissue. In the case of a positive crossmatch, anti-CD40 antibodies may be administered prior to transplantation. In certain embodiments, the transplant is a kidney transplant.

The anti-CD40 antibodies or antigen-binding fragments thereof described herein can also be used in treating or preventing idiopathic thrombocytopenic purpura (ITP) in a patient in need thereof. ITP (also referred to as primary immune thrombocytopenia or primary immune thrombocytopenic purpura or autoimmune thrombocytopenic purpura), is an autoimmune condition with antibodies detectable against several platelet surface antigens. The disease is defined as isolated low platelet count (thrombocytopenia) with normal bone marrow and the absence of other causes of thrombocytopenia. It causes a characteristic purpuric rash and an increased tendency to bleed. Two distinct clinical syndromes manifest as an acute condition in children and a chronic condition in adults. The acute form often follows an infection and has a spontaneous resolution within 2 months. Chronic idiopathic thrombocytopenic purpura persists longer than 6 months without a specific cause.

The antibodies of this disclosure are useful in treating diseases in which autoantibodies, alloantibodies or antibodies against therapeutic proteins are causative of the disease in a patient in need thereof.

The antibodies of this disclosure are also useful in reducing or preventing T cell-dependent antibody responses in a patient in need thereof.

The anti-CD40 antibodies or antigen-binding fragments thereof described herein can also be used in treating or preventing a fibrotic disease in a patient in need thereof. Fibrotic disease results from the excessive deposition of extra cellular matrix (ECM) components such as fibronectin (FN) and type I collagen (Col1α1) by fibroblasts. Organ fibrosis is the final common pathway for many diseases that result in end-stage organ failure. Uncontrollable wound-healing responses, including acute and chronic inflammation, angiogenesis, activation of resident cells, and ECM remodeling, are thought to be involved in the pathogenesis of fibrosis. TGF-β is the prototypic fibrotic cytokine that is increased in fibrosis. It contributes to the development of fibrosis by stimulating the synthesis of ECM molecules, activating fibroblasts to α-smooth muscle actin-expressing myofibroblasts, and downregulating matrix metalloproteinases. The antibodies of this disclosure can be used in treating a patient with a fibrotic disease, such as, but not limited to, scleroderma, lung fibrosis (e.g., idiopathic pulmonary fibrosis, cystic fibrosis, progressive massive fibrosis, or resulting from environmental insults including toxic particles, sarcoidosis, asbestosis, hypersensitivity pneumonitis, bacterial infections including tuberculosis, medicines, etc.), kidney fibrosis (e.g., resulting from chronic inflammation, infections or type II diabetes), liver fibrosis (e.g., cirrhosis, alcoholic, viral, autoimmune, metabolic and hereditary chronic disease), pancreatic fibrosis (e.g., resulting from, for example, alcohol abuse and chronic inflammatory disease of the pancreas), fibrosis of the spleen (from sickle cell anemia, other blood disorders), cardiac fibrosis (e.g., endomyocardial fibrosis, atrial fibrosis, or resulting from infection, inflammation, and hypertrophy), uterine fibrosis, nephrogenic systemic fibrosis, mediastinal fibrosis, myelofibrosis, retroperitoneal fibrosis, progressive massive fibrosis, nephrogenic systemic fibrosis, fibrotic complications of surgery (e.g., especially after surgical implants or scarring after surgery), injection fibrosis, fibrosis as a result of Graft-Versus-Host Disease (GVHD), interstitial fibrosis, subepithelial fibrosis, Crohn's disease, arthrofibrosis, Peyronie's disease, Dupuytren's contracture, Alport's syndrome, morphea, a keloid scar, a hypertrophic scar, aberrant wound healing, glomerulonephritis, and multifocal fibrosclerosis.

A patient (e.g., a human patient) who is at risk for, diagnosed with, or who has one of these disorders can be administered an anti-CD40 antibody or antigen-binding fragment thereof described herein in an amount and for a time to provide an overall therapeutic effect. The anti-CD40 antibody or antigen-binding fragment thereof can be administered alone (monotherapy) or in combination with one or more other agents (combination therapy). Examples of such agents include: an artificial tears supplement, a topical cyclosporine (e.g., cyclosporin A), saliva secretagogue (e.g., pilocarpine), hydroxychloroquine, a systemic corticosteroid, an anti-BAFF antibody (e.g., belimumab), an anti-CD20 antibody (e.g., rituximab), an anti-CD22 antibody (e.g., epratuzumab), an anti-IL6R antibody (e.g., tocilizumab), a lymphotoxin-β receptor fusion protein (e.g., baminercept), an anti-CTLA4 antibody, a CTLA4-Ig protein (e.g., CTLA4-IgGFc fusion molecule (e.g., abatacept)), another anti-CD40 antibody, an anti-CD40L antibody, an anti-B7.1 (CD80) antibody, an anti-B7.2 (CD86) antibody, an agent that targets the B7-CD28 pathway, an anti-lymphotoxin-β receptor antibody, and immunosuppressive agents (e.g., glucocorticoids, cytostatics (alkylating agents (e.g., nitrogen mustards (cyclophosphamide), nitrosoureas, platinum compounds), antimetabolites (e.g., folic acid analogues, purine analogues, pyrimidine analogues, protein synthesis inhibitors)), drugs acting on immunophilins (e.g., cyclosporin, tacrolimus, sirolimus); interferon (e.g., IFN-β, IFN-γ), opioid, TNF binding proteins, mycophenolate (e.g., mycophenolate mofetil, mycophwenolic acid)), mizorubine, deoxyspergualin, brequinar sodium, leflunomide, azaspirane, myriocin, and fingolimod). The amounts and times of administration for combination therapies can be those that provide, e.g., an additive or a synergistic therapeutic effect. Further, the administration of the anti-CD40 antibody (with or without the second agent) can be used as a primary, e.g., first line treatment, or as a secondary treatment, e.g., for subjects who have an inadequate response to a previously administered therapy (i.e., a therapy other than one with an anti-CD40 antibody).

### Pharmaceutical Compositions

An anti-CD40 antibody or antigen-binding fragment thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd}ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, an anti-CD40 antibody or antigen-binding fragment thereof described herein is formulated with excipient materials, such as citrate, arginine, histidine, succinate, methionine, glycine, sorbitol, or polysorbate-80 (Tween-80). It can be provided, for example, in a buffered solution at a suitable concentration and can be stored at 2-8° C. In some other embodiments, the pH of the composition is between about 5.0 and about 6.6 (e.g., 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8).

The pharmaceutical compositions can also include agents that reduce aggregation of the CD40 antibody or antigen-binding fragment thereof when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or a tonicity modifier (e.g., mannitol, or sorbitol) and/or a surfactant (e.g., polysorbate-20 or polysorbate-80).

Such compositions can be administered by a parenteral mode (e.g., intravenous, subcutaneous, intraperitoneal, or intramuscular injection). In one embodiment, the anti-CD40 antibody or antigen-binding fragment thereof composition is administered intravenously. In another embodiment, the anti-CD40 antibody or antigen-binding fragment thereof composition is administered subcutaneously. The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-CD40 antibody or antigen-binding fragment thereof may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In one embodiment, the pharmaceutical formulation comprises an anti-CD40 antibody or antigen-binding fragment thereof (e.g., Exemplary anti-CD40 Antibody 1) at a concentration of about 0.5 mg/mL, to 300 mg/mL (e.g., 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 mg/mL, 250 mg/mL), formulated in a citrate buffer optionally with arginine and/or sucrose. In other embodiments, the anti-CD40 antibody or antigen-binding fragment thereof is formulated in a histidine buffer optionally with arginine and/or sucrose. In a further embodiment, the anti-CD40 antibody or antigen-binding fragment thereof is formulated in a succinate buffer optionally with arginine and/or sucrose. The formulations may also optionally contain methionine and/or Tween-80 (0.01-0.1%, e.g., 0.03%, 0.05%, or 0.7%). The pH of the formulation may be between 5.0 and 7.5 (e.g., 5.0, 5.1, 5.2, 5.3, 5.4, 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4). In certain embodiments, the formulation has a pH of 5-6. In a specific embodiment, the formulation has a pH of 6.0

### Administration

The anti-CD40 antibody or antigen-binding fragment thereof described herein can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment thereof can be administered as a fixed dose, or in a mg/kg dose. The dose can also be chosen to reduce or avoid production of antibodies against the anti-CD40 antibody. Dosage regimens are adjusted to provide the desired response, e.g., a therapeutic response or a combinatorial therapeutic effect. Generally, doses of the anti-CD40 antibody (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In specific embodiments, a subject in need of treatment with an anti-CD40 antibody is administered the antibody at a dose 2 mg/kg, 4 mg/kg, 5 mg/kg, 10 mg/kg, 15 mg/kg, 20 mg/kg, 30 mg/kg, 35 mg/kg, or 40 mg/kg.

A composition may comprise about 1 mg/mL to 100 mg/ml or about 10 mg/mL to 100 mg/mL or about 50 to 250 mg/mL or about 100 to 150 mg/mL or about 100 to 250 mg/mL of anti-CD40 antibody or antigen-binding fragment thereof.

In certain embodiments, the anti-CD40 antibody or antigen-binding fragment thereof in a composition is predominantly in monomeric form, e.g., at least about 90%, 92%, 94%, 96%, 98%, 98.5% or 99% in monomeric form. Certain anti-CD40 antibody or antigen-binding fragment thereof compositions may comprise less than about 5, 4, 3, 2, 1, 0.5, 0.3 or 0.1% aggregates, as detected, e.g., by UV at A280 nm. Certain anti-CD40 antibody or antigen-binding fragment thereof compositions comprise less than about 5, 4, 3, 2, 1, 0.5, 0.3, 0.2 or 0.1% fragments, as detected, e.g., by UV at A280 nm.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-CD40 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. In one embodiment, the anti-CD40 antibody or antigen-binding fragment thereof described herein is administered biweekly. In a specific embodiment, the anti-CD40 antibody or antigen-binding fragment thereof described herein is administered monthly. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

If a subject is at risk for developing an immunological disorder described herein, the antibody can be administered before the full onset of the immunological disorder, e.g., as a preventative measure. The duration of such preventative treatment can be a single dosage of the antibody or the treatment may continue (e.g., multiple dosages). For example, a subject at risk for the disorder or who has a predisposition for the disorder may be treated with the antibody for days, weeks, months, or even years so as to prevent the disorder from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In certain embodiments, the anti-CD40 antibody or antigen-binding fragment thereof is administered subcutaneously at a concentration of about 1 mg/mL to about 300 mg/mL 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 mg/mL, 250 mg/mL). In one embodiment, the anti-CD40 antibody or antigen-binding fragment thereof is administered subcutaneously at a concentration of 50 mg/mL. In a different embodiment, the anti-CD40 antibody or antigen-binding fragment thereof is administered subcutaneously at a concentration of 150 mg/mL. In another embodiment, the anti-CD40 antibody or antigen-binding fragment thereof is administered subcutaneously at a concentration of 200 mg/mL. In another embodiment, the anti-CD40 antibody or antigen-binding fragment thereof is administered intravenously at a concentration of about 1 mg/mL to about 300 mg/mL 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 mg/mL, 250 mg/mL). In a particular embodiment, the anti-CD40 antibody or antigen-binding fragment thereof is administered intravenously at a concentration of 50 mg/mL. In one embodiment, the anti-CD40 antibody or antigen-binding fragment thereof is administered intravenously at a concentration of 75 mg/mL. The administration can be e.g., biweekly or monthly.

### Devices and Kits for Therapy

Pharmaceutical compositions that include the anti-CD40 antibody or antigen-binding fragment thereof can be administered with a medical device. The device can be designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include anti-CD40 antibody or antigen-binding fragment thereof, and can be configured to deliver one or more unit doses of the antibody. The device can be further configured to administer a second agent (e.g., an artificial tears supplement, a topical cyclosporine (e.g., cyclosporin A), saliva secretagogue (e.g., pilocarpine), hydroxychloroquine, a systemic corticosteroid, an anti-BAFF antibody (e.g., belimumab), an anti-CD20 antibody (e.g., rituximab), an anti-CD22 antibody (e.g., epratuzumab), an anti-IL6R antibody (e.g., tocilizumab), a lymphotoxin-β receptor fusion protein (e.g., baminercept), an anti-CTLA4 antibody, a CTLA4-Ig protein (e.g., CTLA4-IgGFc fusion molecule (e.g., abatacept)), another anti-CD40 antibody, an anti-CD40L antibody, an anti-B7.1 (CD80) antibody, an anti-B7.2 (CD86) antibody, an agent that targets the B7-CD28 pathway, an anti-lymphotoxin-β receptor antibody, and immunosuppressive agents (e.g., glucocorticoids, cytostatics (alkylating agents (e.g., nitrogen mustards (cyclophosphamide), nitrosoureas, platinum compounds), antimetabolites (e.g., folic acid analogues, purine analogues, pyrimidine analogues, protein synthesus inhibitors)), drugs acting on immunophilins (e.g., ciclosporin, tacrolimus, sirolimus); interferon (e.g., IFN-β, IFN-γ), opioid, TNF binding proteins, mycophenolate (e.g., mycophenolate mofetil, mycophwenolic acid)), mizorubine, deoxyspergualin, brequinar sodium, leflunomide, azaspirane, myriocin, and fingolimod), either as a single pharmaceutical composition that also includes the anti-CD40 antibody or antigen-binding fragment thereof or as two separate pharmaceutical compositions.

The pharmaceutical composition may be administered with a syringe. The pharmaceutical composition can also be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-CD40 antibody or antigen-binding fragment thereof can be provided in a kit. In one embodiment, the kit includes (a) a container that contains a composition that includes anti-CD40 antibody, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein (e.g., an artificial tears supplement, a topical cyclosporine (e.g., cyclosporin A), saliva secretagogue (e.g., pilocarpine), hydroxychloroquine, asystemic corticosteroid, an anti-BAFF antibody (e.g., belimumab), an anti-CD20 antibody (e.g., rituximab), an anti-CD22 antibody (e.g., epratuzumab), an anti-IL6R antibody (e.g., tocilizumab), a lymphotoxin-β receptor fusion protein (e.g., baminercept), an anti-CTLA4 antibody, a CTLA4-IgGFc fusion molecule (e.g., abatacept), another anti-CD40 antibody, an anti-CD40L antibody). For example, the kit includes a first container that contains a composition that includes the anti-CD40 antibody, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-CD40 antibody or antigen-binding fragment thereof, e.g., in a suitable dose, dosage form, or mode of administration (e.g., a dose, dosage form, or mode of administration described herein), to treat a subject who has had or who is at risk for an immunological disorder described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-CD40 antibody or antigen-binding fragment thereof and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Cloning of the Heavy and Light Chains of Murine Anti-CD40 Antibody

The AKH3 murine hybridoma was derived from an RBF mouse immunized with a complex of CD40/CD40L extracellular domain (ECD) constructs. Splenocytes from one mouse were fused to FL653 myeloma cells resulting in a hybridoma that produced the AKH3 antibody. AKH3 was demonstrated to be specific for binding to human CD40 and capable of blocking the interaction with CD40L.

The AKH3 hybridoma was cultured and frozen cell pellets were prepared for RNA isolation. Total cellular RNA was isolated from the AKH3 cell pellets using the Qiagen RNeasy mini kit. cDNAs encoding the AKH3 heavy and AKH3 light chain variable domains were generated by RT-PCR with random hexamers (GE Healthcare First Strand cDNA Synthesis kit). Specific PCR amplification of the murine immunoglobulin gene family, including the signal sequences, was performed in two separate reactions. The reaction for the heavy chain sequences was accomplished using a cocktail of oligonucleotide primers within the signal peptide sequence and a single oligonucleotide primer within the constant domain. Similarly, the reaction for the light chain sequences was accomplished using a cocktail of oligonucleotide primers within the signal peptide sequence and a single oligonucleotide primer within the kappa domain (Table 1).

TABLE 1
Oligonucleotide Sequences for RT-PCR

| Function | Name | Sequence |
|---|---|---|
| VH 1 MIX | OT3-316 | ACTAGTCGACATGAAATGCAGCTGGGTCATSTTCTTC SEQ ID |
| | | NO: 1) |
| | OT3-317 | ACTAGTCGACATGGGATGGAGCTRTATCATSYTCTT (SEQ ID |
| | | NO: 2) |
| | OT3-318 | ACTAGTCGACATGAAGWTGTGGTTAAACTGGGTTTTT (SEQ ID |
| | | NO: 3) |
| | OT3-319 | ACTAGTCGACATGRACTTTGGGYTCAGCTTGRTTT (SEQ ID |
| | | NO: 4) |
| | OT3-320 | ACTAGTCGACATGGACTCCAGGCTCAATTTAGTTTTCCTT (SEQ |
| | | ID NO: 5) |
| | OT3-321 | ACTAGTCGACATGGCTGTCYTRGSGCTRCTCTTCTGC (SEQ ID |
| | | NO:6) |
| VH 1 reverse | CDL 739 | AGGTCTAGAAYCTCCACACACAGGRRCCAGTGGATAGAC (SEQ |
| | | ID NO: 7) |
| VH 2 MIX | OT3-322 | ACTAGTCGACATGGRATGGAGCKGGRTCTTTMTCTT (SEQ ID |
| | | NO: 8) |
| | OT3-323 | ACTAGTCGACATGAGAGTGCTGATTCTTTTGTG (SEQ ID |
| | | NO: 9) |
| | OT3-324 | ACTAGTCGACATGGMTTGGGTGTGGAMCTTGCTATTCCTG (SEQ |
| | | ID NO: 10) |
| | OT3-325 | ACTAGTCGACATGGGCAGACTTGCATTCTCATTCCTG (SEQ ID |
| | | NO: 11) |
| | OT3-326 | ACTAGTCGACATGGATTTTGGGCTGATTTTTTTTATTG (SEQ |
| | | ID NO: 12) |
| | OT3-327 | ACTAGTCGACATGATGGTGTTAAGTCTTCTGTACCTG (SEQ ID |
| | | NO: 13) |
| VH 2 reverse | CDL-739 | AGGTCTAGAAYCTCCACACACAGGRRCCAGTGGATAGAC (SEQ |
| | | ID NO: 14) |
| V kappa 1 MIX | OT3-174 | ACTAGTCGACATGAAGTTGCCTGTTAGGCTGTTGGTGCTG (SEQ |
| | | ID NO: 15) |
| | OT3-175 | ACTAGTCGACATGGAGWCAGACACACTCCTGYTATGGGT (SEQ |
| | | ID NO: 16) |
| | OT3-176 | ACTAGTCGACATGAGTGTGCTCACTCAGGTCCTGGSGTTG (SEQ |
| | | ID NO: 17) |
| | OT3-177 | ACTAGTCGACATGAGGRCCCCTGCTCAGWTTYTTGGMWTCTTG |
| | | (SEQ ID NO: 18) |
| | OT3-178 | ACTAGTCGACATGGATTTWCAGGTGCAGATTWTCAGCTTC (SEQ |
| | | ID NO: 19) |
| | OT3-179 | ACTAGTCGACATGAGGTKCYYTGYTSAGYTYCTGRGG (SEQ ID |
| | | NO: 20) |
| V kappa 1 | CDL-738 | GCGTCTAGAACTGGATGGTGGGAGATGGA (SEQ ID NO: 21) |
| reverse | | |
| V kappa 2 MIX | OT3-180 | ACTAGTCGACATGGGCWTCAAGATGGAGTCACAKWYYCWGG |
| | | (SEQ ID NO: 22) |
| | OT3-181 | ACTAGTCGACATGTGGGGAYCTKTTTYCMMTTTTTCAATTG |
| | | (SEQ ID NO: 23) |
| | OT3-182 | ACTAGTCGACATGGTRTCCWCASCTCAGTTCCTTG (SEQ ID |
| | | NO: 24) |
| | OT3-183 | ACTAGTCGACATGTATATATGTTTGTTGTCTATTTCT (SEQ ID |
| | | NO: 25) |
| | OT3-184 | ACTAGTCGACATGGAAGCCCCAGCTCAGCTTCTCTTCC (SEQ |
| | | ID NO: 26) |
| V kappa 2 | CDL-738 | GCGTCTAGAACTGGATGGTGGGAGATGGA (SEQ ID NO: 27) |
| reverse | | |

PCR products were gel-purified and cloned into the pCR2.1 TOPO vector (Invitrogen). The plasmids were transformed into E. coli, and the plasmid DNA from multiple colonies was subjected to DNA sequence analysis. Sequences were aligned to establish a consensus sequence. The variation in the sequences among the clones was consistent with the primer degeneracy. The heavy chain isolate consensus sequence is presented below with the Kabat-complementarity-determining regions (CDRs) underlined.

| (SEQ ID NO: 28) | |
|---|---|
| 1 | QVQLQQSGAE LVKPGASVHM SCKAFGYTFT TFPIEWMRQI HGKSLEWIGN |
| 51 | FHPYNDDTKY NEKFKGKAHL TVEKSSSTVY LELSRLTSDD SAVYYCARRG |
| 101 | KLPFDSWGQG TTLTVSS |

The light chain isolate consensus sequence is presented below with the Kabat-complementarity-determining regions (CDRs) underlined.

| (SEQ ID NO: 29) | |
|---|---|
| 1 | DIQMTQTTSS LSASLGDRVT ISCRASQDIS NYLNWYQQKP DGTVKLLIYF |
| 51 | TSRLRSGVPS RFSGSGSGTD YSLTISNLEP EDIATYYCQQ DRKLPWTFGG |
| 101 | GTKLEIK |

### Example 2. Chimerization of the Murine AKH3 Antibody

Chimeric antibody genes were designed and constructed by joining PCR-amplified variable domains (with suitable transcriptional and translational elements) with human immunoglobulin constant domains or human kappa domain sequences into the pV90 and pV100 vectors (U.S. Pat. No. 7,494,805). The pV90 vector encodes a dihydrofolate reductase marker for selection in CHO DG44 (dhfr-deficient) cells. The pV100 vector encodes a neomycin phosphotransferase gene for selection in the presence of G418.

The chimeric heavy chain sequence was cloned as a human IgG1 chimera in plasmid pYL789 to create a fully Fc effector competent form of AKH3 (chAKH3 IgG1), as well as an aglycosyl human IgG4P/IgG1 chimera in plasmid pYL805 to create the most Fc effectorless form of AKH3 (agly chAKH3) for initial testing. The chimeric light chain sequence was cloned as a human kappa in pYL790.

The sequence of the mature chimeric AKH3-human IgG1 protein is shown below.

| (SEQ ID NO: 30) | |
|---|---|
| 1 | QVQLQQSGAE LVKPGASVKM SCKAFGYTFT TFPIEWMRQI HGKSLEWIGN |
| 51 | FHPYNDDTKY NEKFKGKAHL TVEKSSSTVY LELSRLTSDD SAVYYCARRG |
| 101 | KLPFDSWGQG TTLTVSSAST KGPSVFPLAP SSKSTSGGTA ALGCLVKDYF |
| 151 | PEPVTVSWNS GALTSGVHTF PAVLQSSGLY SLSSVVTVPS SSLGTQTYIC |
| 201 | NVNHKPSNTK VDKKVEPKSC DHTHTCPPCP APELLGGPSV FLFPPKPKDT |
| 251 | LMISRTPEVT CVVVDVSHED PEVKFNWYVD GVEVHNAKTH PREEQYNSTY |
| 301 | RVVSVLTVLH QDWLNGKEYK CKVSNHALPA PIEKTISKAK GQPREPQVYT |
| 351 | LPPSRDELTK NQVSLTCLVK GFYPSDIAVE WESNGQPENN YKTTPPVLDS |
| 401 | DGSFFLYSKL TVOKSRWQQG NVFSCSVMHE ALHNHYTQKS LSLSPG |

The sequence of the mature chimeric AKH3-human IgG4P/IgG1 protein is shown below.

| (SEQ ID NO: 31) | |
|---|---|
| 1 | QVQLQQSGAE LVKPGASVKM SCKAFGYTFT TFPIEWMRQI HGKSLEWIGN |
| 51 | FHPINDDTKY NEKFKGKAKL TVEKSSSTVY LELSRLTSDD SAVYYCARRG |
| 101 | KLPFDSWGQG TTLTVSSAST KGPSVFPLAP CSRSTSESTA ALGCLVKDYF |
| 151 | PEPVTVSWNS GALTSGVHTF PAVLQSSGLY SLSSVVTVPS SSLGTKTYTC |
| 201 | NVDHKPSNTK VDKRVESKYG PPCPPCPAPE FLGGPSVFLF PPHPKDTLMI |
| 251 | SRTPEVTCVV VDVSQEDPEV QFNWYVDGVE VHNAKTKPRE EQFQSTYRVV |
| 301 | SVLTVLHQDW LNGKEYKCKV SNKGLPSSIE KTISKAKGQP REPQVYTLPP |
| 351 | SRDELTKNQV SLTCLVKGFY PSDIAVEWES NGQPENNYKT TPPVLDSDGS |
| 401 | FFLYSKLTVD KSRWQQGNVF SCSVMHEALH NHYTQKSLSL SPG |

The sequence of the mature chimeric AKH3-human Kappa protein is shown below.

| (SEQ ID NO: 32) | |
|---|---|
| 1 | DIQMTQTTSS LSASLGDRVT ISCRASQDIS NYLNWYQQKP DGTVKLLIYF |
| 51 | TSRLRSGVFS RFSGSGSGTD YSLTISNLEF EDIATYYCQQ DRKLFWTFGG |
| 101 | GTKLEIKRTV AAPSVFIFPF SDEQLKSGTA SVVCLLNNFY FREAKVQWKV |
| 151 | DNALQSGNSQ ESVTEQDSKD STYSLSSTLT LSKADYEKHK VYACEVTHQG |
| 201 | LSSPVTKSFN RGEC |

For screening purposes, the chimeric versions of AKH3 were produced by transfection of heavy chain (HC) and light chain (LC) pairs of plasmids into HEK293-EBNA (293E) cells. Conditioned media were tested for antibody secretion and specificity. Western blot analysis confirmed that chimeric AKH3-transfected cells efficiently synthesized heavy and light chain proteins and that they assembled into the HC2LC2 tetrameric unit characteristic of antibodies. Direct FACS binding to human CD40 expressed on the surface of 293E cells confirmed that the constructs were functional. The EC₅₀ values for the chimeric AKH3 were similar to the EC₅₀ for the murine hybridoma AKH3.

For production purposes, stable pools of CHO cells expressing chimeric AKH3 huIgG1 (chAKH3 IgG1) and chimeric aglycosyl AKH3 huIgG4P/IgG1 (agly chAKH3) were generated by transfection of plasmids encoding these chimeric antibodies.

### Example 3. Humanization of the Heavy and Light Chains of AKH3

In preparation for humanization, the AKH3 variable domains were examined for the boundaries of the CDRs, most-similar murine germ-line sequences, most-similar human germ-line sequences, and other potentially undesirable protein motifs (e.g. N-linked glycosylation, acid-sensitive, cleavage sites, etc.). The variable domains of the heavy chain and light chain sequences of the AKH3 antibody are presented in Example 1. The heavy chain sequence in AKH3 is most likely a murine subgroup Heavy II (A) derived from germline J558.52. The closest match with human germ line sequence is IGHV1-3*01. The light chain sequence in AKH3 is most likely a murine subgroup Kappa V originating from germline IGKV10-94. The closest match with human germ line sequence is IGKV1-27.

Modeling was performed using a variety of analytical tools and the output was a series of heavy chain and light chain designs. For the heavy chain, the CDR graft, designated H0 was not recommended for testing because five back mutations made this framework more like a human VH1 germline than a straight CDR graft would be. The five back mutations, E6Q, S16A, N84S, S85R, K109Q, were incorporated into design H1. A total of five designs with an increasing number of back-mutations named H1, H2, H3, H4, and H5 were generated and tested in combination with each of the four light chain designs and design H1 was selected. The variable domain of the humanized AKH3 variant H1 is shown below with underlined CDRs and the changes from a CDR graft are highlighted.

| (SEQ ID NO: 33) | |
|---|---|
| 1 | 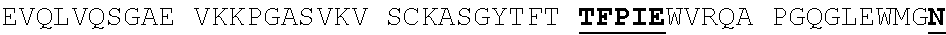 |
| 51 | 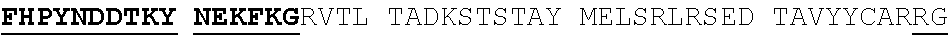 |
| 101 | 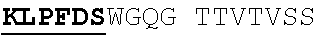 |

For the light chain, the CDR graft (L0) was recommended for testing. Two back mutations were made to L0 at T22S and F71Y to generate design L1. A total of three light chain designs named L0, L1, and L2 were generated and tested in combination with each of the five heavy chain designs and L1 was selected. The variable domain of the humanized AKH3 variant L1 is shown below with underlined CDRs and the changes from a CDR graft are highlighted.

| (SEQ ID NO: 34) | |
|---|---|
| 1 | 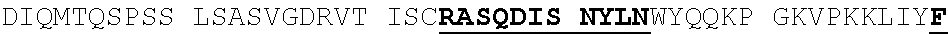 |
| 51 | 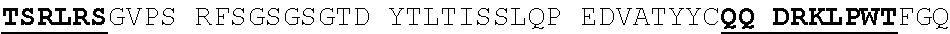 |
| 101 | GTKLEIK |

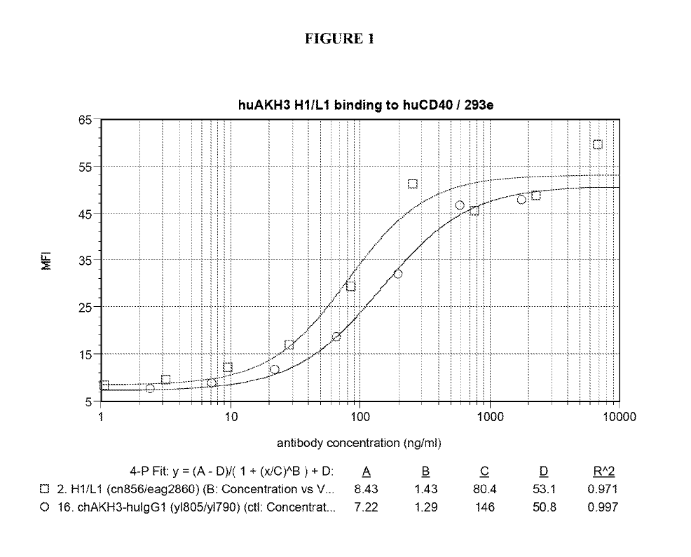

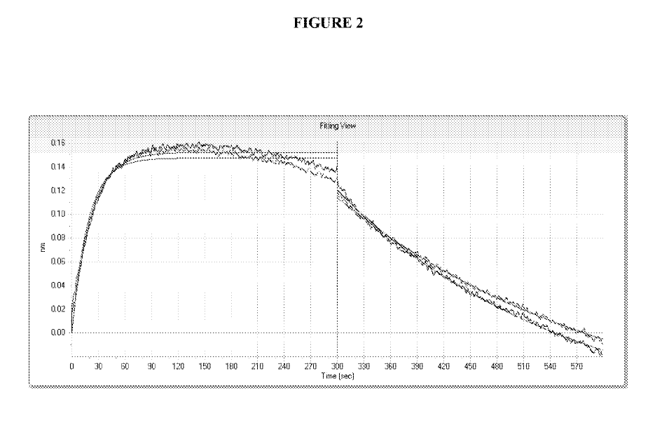

Full length antibodies were engineered using the humanized variable domain designs. The heavy chains were cloned as effectorless, human aglycosyl IgG4 S225P N294Q/IgG1 and the light chains were cloned as human kappa. Five heavy chain variants (H1-H5) and three light chain variants (L0-L2) were paired and transfected into 293 EBNA (293E) cells as an array of 15 transfections. The conditioned media from all of the combinations of heavy and light chain variants were screened for bivalent binding to cell surface CD40 by FACS and for monomeric binding to soluble CD40 by Octet resulting in selection of the H1L1 humanized variant. As shown in FIGS. 1 and 2, there was no loss of CD40 binding with the selected H1L1, humanized antibody as compared to the original murine AKH3 hybridoma variable domains.

The aglycosyl AKH3 H1-IgG4 S225P N294Q/IgG1 heavy chain was expressed in combination with the AKH3 L1 light chain in stably transfected CHO cells for further characterization of this effectorless version. The DNA sequence and translated amino acid sequence of H1-aglycosyl IgG4 S225P N294Q/IgG1 are shown below.

| | |
|---|---|
| 1 | atg ggt tgg agc ctc atc ttg ctc ttc ctt gtc gct gtt gct acg cgt gtc ctg tcc |
| 1> | M G W S L I L L F L V A V A T R V L S |
| 58 | GAG GTT CAG CTG GTG CAG TCT GGG GCT GAG GTC AAG AAG CCT GGG GCC TCA GTG AAG |
| 20> | E V Q L V Q S G A E V K K P G A S V K |
| 115 | GTG TCC TGC AAG GCT AGC GGT TAC ACC TTC ACT ACC TTT CCA ATC GAG TGG GTT AGG |
| 39> | V S C K A S G Y T F T T F P I E W V R |
| 172 | CAG GCT CCA GGA CAG GGT CTA GAG TGG ATG GGA AAT TTT CAT CCT TAC AAT GAT GAT |
| 58> | Q A P G Q G L E W M G N F H P Y N D D |
| 229 | ACT AAG TAC AAT GAA AAA TTC AAG GGC AGG GTC ACA TTG ACT GCC GAT AAG TCC ACC |
| 77> | T K Y N E K F K G R V T L T A D K S T |
| 286 | AGC ACA GCT TAC ATG GAG CTC AGC CGA TTA AGG TCT GAA GAC ACA GCT GTT TAT TAC |
| 96> | S T A Y M E L S R L R S E D T A V Y Y |
| 343 | TGT GCA AGG CGG GGT AAA CTA CCC TTT GAC TCC TGG GGC CAA GGC ACC ACT GTG ACA |
| 115> | C A R R G K L P F D S W G Q G T T V T |
| 400 | GTC TCC TCA GCT TCC ACC AAG GGC CCA TCC GTC TTC CCC CTG GCG CCC TGC TCC AGA |
| 134> | V S S A S T K G P S V F P L A P C S R |
| 457 | TCT ACC TCC GAG AGC ACA GCC GCC CTG GGC TGC CTG GTC AAG GAC TAC TTC CCC GAA |
| 153> | S T S E S T A A L G C L V K D Y F P E |
| 514 | CCG GTG ACG GTG TCG TGG AAC TCA GGC GCC CTG ACC AGC GGC GTG CAC ACC TTC CCG |
| 172> | P V T V S W N S G A L T S G V H T F P |
| 571 | GCT GTC CTA CAG TCC TCA GGA CTC TAC TCC CTC AGC AGC GTG GTG ACC GTG CCC TCC |
| 191> | A V L Q S S G L Y S L S S V V T V P S |
| 528 | AGC AGC TTG GGC ACG AAG ACC TAC ACC TGC AAC GTA GAT CAC AAG CCC AGC AAC ACC |
| 210> | S S L G T K T Y T C N V D H K P S N T |
| 685 | AAG GTG GAC AAG AGA GTT GAG TCC AAA TAT GGT CCC CCA TGC CCA CCG TGC CCA GCA |
| 229> | K V D K R V E S K Y G P P C P P C P A |
| 742 | CCT GAG TTC CTG GGG GGA CCA TCA GTC TTC CTG TTC CCC CCA AAA CCC AAG GAC ACT |
| 248> | P E F L G G P S V F L F P P K P K D T |
| 799 | CTC ATG ATC TCC CGG ACC CCT GAG GTC ACG TGC GTG GTG GTG GAC GTG AGC CAG GAA |
| 267> | L M I S R T P E V T C V V V D V S Q E |
| 856 | GAC CCC GAG GTC CAG TTC AAC TGG TAC GTG GAT GGC GTG GAG GTG CAT AAT GCC AAG |
| 286> | D P E V Q F N W Y V D G V E V H N A K |
| 913 | ACA AAG CCG CGG GAA GAG CAG TTC CAG AGC ACG TAC CGT GTG GTC AGC GTC CTC ACC |
| 305> | T K P R E E Q F Q S T Y R V V S V L T |
| 970 | GTC CTG CAC CAG GAC TGG CTG AAC GGC AAG GAG TAC AAG TGC AAG GTC TCC AAC AAA |
| 324> | V L H Q D W L N G K E Y K C K V S N K |
| 1027 | GGC CTC CCG TCC TCC ATC GAG AAA ACC ATC TCC AAA GCC AAA GGG CAG CCC CGA GAG |
| 343> | G L P S S I E K T I S K A K G Q P R E |
| 1084 | CCA CAA GTG TAC ACC CTG CCC CCA TCC CGG GAT GAG CTG ACC AAG AAC CAG GTC AGC |
| 362> | P Q V Y T L P P S R D E L T K N Q V S |
| 1141 | CTG ACC TGC CTG GTC AAA GGC TTC TAT CCC AGC GAC ATC GCC GTG GAG TGG GAG AGC |
| 381> | L T C L V K G F Y P S D I A V E W E S |
| 1198 | AAT GGG CAG CCG GAG AAC AAC TAC AAG ACC ACG CCT CCC GTG TTG GAC TCC GAC GGC |
| 400> | N G Q P E N N Y K T T P P V L D S D G |
| 1255 | TCC TTC TTC CTC TAC AGC AAG CTC ACC GTG GAC AAG AGC AGG TGG CAG CAG GGG AAC |
| 419> | S F F L Y S K L T V D K S R W Q Q G N |
| 1312 | GTC TTC TCA TGC TCC GTG ATG CAT GAG GCT CTG CAC AAC CAC TAC ACG CAG AAG AGC |
| 438> | V F S C S V M H E A L H N H Y T Q K S |
| 1369 | CTC TCC CTG TCT CCC GGT TGA (SEQ ID NO: 35) |
| 457> | L S L S P G * (SEQ ID NO: 36) |

Amino acids 1-462 contain the heavy chain sequence. Amino acids 1-19 (nucleotides in lower case) contain the synthetic heavy chain signal peptide. The mature N-terminus begins with amino acid 20 (E).

The DNA sequence and translated amino acid sequence of anti-CD40 L1 Kappa are shown below. Amino acids 1-236 contain the light chain sequence. Amino acids 1-22 (nucleotides in lower case) contain the native human kappa light chain signal peptide. The mature N-terminus begins with amino acid 23 (D).

| | |
|---|---|
| 1 | atg gac atg agg gtc ccc gct cag ctc ctg ggg ctc ctt ctg ctc tgg ctc cct gga |
| 1> | M D M R V P A Q L L G L L L L W L P G |
| 58 | gca cga tgt GAT ATC CAG ATG ACA CAG AGC CCT TCC TCC CTG TCT GCC TCT GTC GGA |
| 20> | A R C D I Q M T Q S P S S L S A S V G |
| 115 | GAC AGG GTC ACC ATT TCC TGC CGC GCA AGT CAG GAC ATT AGC AAT TAT TTA AAC TGG |
| 39> | D R V T I S C R A S Q D I S N Y L N W |
| 172 | TAT CAA CAG AAA CCA GGC AAG GTC CCT AAA CTC CTG ATC TAC TTC ACA TCA AGA TTA |
| 58> | Y Q Q K P G K V P K L L I Y F T S R L |
| 229 | CGC TCA GGA GTC CCA TCA AGG TTC AGT GGC AGT GGG TCT GGG ACA GAT TAT ACC CTC |
| 77> | R S G V P S R F S G S G S G T D Y T L |
| 286 | ACC ATT AGC TCT CTG CAA CCG GAA GAC GTG GCC ACT TAC TAT TGC CAA CAG GAT CGG |
| 96> | T I S S L Q P E D V A T Y Y C Q Q D R |
| 343 | AAA CTT CCG TGG ACG TTC GGT CAG GGC ACC AAG CTG GAA ATC AAG CGT ACG GTG GCT |
| 115> | K L P W T F G Q G T K L E I K R T V A |
| 400 | GCA CCA TCT GTC TTC ATC TTC CCG CCA TCT GAT GAG CAG TTG AAA TCT GGA ACT GCC |
| 134> | A P S V F I F P P S D E Q L K S G T A |
| 457 | TCT GTT GTG TGC CTG CTG AAT AAC TTC TAT CCC AGA GAG GCC AAA GTA CAG TGG AAG |
| 153> | S V V C L L N N F Y P R E A K V Q W K |
| 514 | GTG GAT AAC GCC CTC CAA TCG GGT AAC TCC CAG GAG AGT GTC ACA GAG CAG GAC AGC |
| 172> | V D N A L Q S G N S Q E S V T E Q D S |
| 571 | AAG GAC AGC ACC TAC AGC CTC AGC AGC ACC CTG ACG CTG AGC AAA GCA GAC TAC GAG |
| 191> | K D S T Y S L S S T L T L S K A D Y E |
| 628 | AAA CAC AAA GTC TAC GCC TGC GAA GTC ACC CTG ACG CTG AGC AAA GCA GAC TAC GAG |
| 210> | K H K V Y A C E V T H Q G L S S P V T |
| 685 | AAG AGC TTC AAC AGG GGA GAG TGT TAG (SEQ ID NO: 37) |
| 229> | K S F N R G E C * (SEQ ID NO: 38) |

After extensive characterization, the agly AKH3 IgG4P/IgG1 was not further pursued due to agonism that was observed with this effectorless version in whole blood assays and a glycosylated human IgG4P version was pursued. The lower agonism exhibited by the IgG4P version of the antibody when compared to the agly IgG4P/IgG1 version of the antibody was surprising. The AKH3 H1 heavy chain gene was recloned as a human IgG4 S225P (S228P Kabat numbering) molecule. The mature AKH3 H1-IgG4 S225P (AKH3 IgG4P) amino acid sequence is provided below (the Kabat CDRs are underlined and the S225P change at position 225 in the sequence below is highlighted, italicized, and underlined).

| (SEQ ID NO: 39) | |
|---|---|
| 1 | EVQLVQSGAE VKKPGASVKV SCKASGYTFT TFPIEWVRQA PGQGLEWMGN |
| 51 | FHPYNDDTKY NEKFKGRVTL TADKSTSTAY MELSRLRSED TAVYYCARRG |
| 101 | KLPFDSWGQG TTVTVSSAST KGPSVFPLAP CSRSTSESTA ALGCLVHDYF |
| 151 | PEPVTVSWNS GALTSGVHTF PAVLQSSGLY SLSSVVTVPS SSLGTKTYTC |
| 201 | 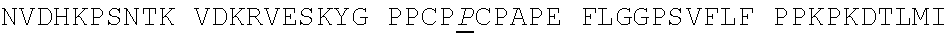 |
| 251 | SRTPEVTCVV VDVSQEDPEV QFNWYVDGVE VHNAKTKPRE EQFNSTYRVV |
| 301 | SVLTVLHQDW LNGKEYKCKV SNKGLPSSIE KTISKAKGQP REPQVYTLPP |
| 351 | SQEEMTKNQV SLTCLVKGFY PSDIAVEWES NGQPENNYKT TPPVLDSDGS |
| 401 | FFLYSRLTVD KSRWQEGNVF SCSVMHEALH NHYTQKSLSL SLG |

The heavy chain coding sequence including the synthetic signal peptide is provided below. Amino acids 1-462 contain the heavy chain sequence. Amino acids 1-19 (nucleotides in lower case) contain the synthetic heavy chain signal peptide. The mature N-terminus begins with amino acid 20 (E).

| | |
|---|---|
| 1 | atg ggt tgg agc ctc atc ttg ctc ttc ctt gtc gct gtt gct acg cgt gtc ctg tcc |
| 1> | M G W S L I L L F L V A V A T R V L S |
| 58 | GAG GTT CAG CTG GTG CAG TCT GGG GCT GAG GTC AAG AAG CCT GGG GCC TCA GTG AAG |
| 20> | E V Q L V Q S G A E V K K P G A S K V |
| 115 | GTG TCC TGC AAG GCT AGC GGT TAC ACC TTC ACT ACC TTT CCA ATC GAG TGG GTT AGG |
| 39> | V S C K A S G Y T F T T F P I E W V R |
| 172 | CAG GCT CCA GGA CAG GGT CTA GAG TGG ATG GGA AAT TTT CAT CCT TAC AAT GAT GAT |
| 58> | Q A P G Q G L E W M G N F H P Y N D D |
| 229 | ACT AAG TAC AAT GAA AAA TTC AAG GGC AGG GTC ACA TTG ACT GCC GAT AAG TCC ACC |
| 77> | T K Y N E K F K G R V T L T A D K S T |
| 286 | AGC ACA GCT TAC ATG GAG CTC AGC CGA TTA AGG TCT GAA GAC ACA GCT GTT TAT TAC |
| 96> | S T A Y M E L S R L R S E D T A V Y Y |
| 343 | TGT GCA AGG CGG GGT AAA CTA CCC TTT GAC TCC TGG GGC CAA GGC ACC ACT GTG ACA |
| 115> | C A R R G K L P F D S W G Q G T T V T |
| 400 | GTC TCC TCA GCT TCC ACC AAG GGC CCA TCC GTC TTC CCC CTG GCG CCC TGC TCC AGA |
| 134> | V S S A S T K G P S V F P L A P C S R |
| 457 | TCT ACC TCC GAG AGC ACA GCC GCC CTG GGC TGC CTG GTC AAG GAC TAC TTC CCC GAA |
| 153> | S T S E S T A A L G C L V K D Y F P E |
| 514 | CCG GTG ACG GTG TCG TGG AAC TCA GGC GCC CTG ACC AGC GGC GTG CAC ACC TTC CCG |
| 172> | P V T V S W N S G A L T S G V H T F P |
| 571 | GCT GTC CTA CAG TCC TCA GGA CTC TAC TCC CTC AGC AGC GTG GTG ACC GTG CCC TCC |
| 191> | A V L Q S S G L Y S L S S V V T V P S |
| 628 | AGC AGC TTG GGC ACG AAG ACC TAC ACC TGC AAC GTA GAT CAC AAG CCC AGC AAC ACC |
| 210> | S S L G T K T Y T C N V D H K P S N T |
| 685 | AAG GTG GAC AAG AGA GTT GAG TCC AAA TAT GGT CCC CCA TGC CCA CCA TGC CCA GCA |
| 229> | K V D K R V E S K Y G P P C P P C P A |
| 742 | CCT GAG TTC CTG GGG GGA CCA TCA GTC TTC CTG TTC CCC CCA AAA CCC AAG GAC ACT |
| 248> | P E F L G G P S V F L F P P K P K D T |
| 799 | CTC ATG ATC TCC CGG ACC CCT GAG GTC ACG TGC GTG GTG GTG GAC GTG AGC CAG GAA |
| 267> | L M I S R T P E V T C V V V D V S Q E |
| 856 | GAC CCC GAG GTC CAG TTC AAC TGG TAC GTG GAT GGC GTG GAG GTG CAT AAT GCC AAG |
| 286> | D P E V Q F N W Y V D G V E V H N A K |
| 913 | ACA AAG CCG CGG GAG GAG CAG TTC AAC AGC ACG TAC CGT GTG GTC AGC GTC CTC ACC |
| 305> | T K P R E E Q F N S T Y R V V S V L T |
| 907 | GTC CTG CAC CAG GAC TGG CTG AAC GGC AAG GAG TAC AAG TGC AAG GTC TCC AAC AAA |
| 324> | V L H Q D W L N G K E Y K C K V S N K |
| 1027 | GGC CTC CCG TCC TCC ATC GAG AAA AAC ATC TCC AAA GCC AAA GGG CAG CCC CGA GAG |
| 343> | G L P S S I E K T I S K A K G Q P R E |
| 1084 | CCA CAA GTG TAC ACC CTG CCC CCA TCC CAG GAG GAG ATG ACC AAG AAC CAG GTC AGC |
| 362> | P Q V Y T L P P S Q E E M T K N Q V S |
| 1141 | CTG ACC TGC CTG GTC AAA GGC TTC TAC CCC AGC GAC ATC GCC GTG GAG TGG GAG AGC |
| 381> | L T C L V K G F Y P S D I A V E W E S |
| 1198 | AAT GGG CAG CCG GAG AAC AAC TAC AAG ACC ACG CCT CCC GTC CTC GAT TCC GAC GGC |
| 400> | N G Q P E N N Y K T T P P V L D S D G |
| 1255 | TCC TTC TTC CTC TAC AGC AGG CTA ACC GTG GAC AAG AGC AGG TGG CAG GAG GGG AAT |
| 419> | S F F L Y S R L T V D K S R W Q E G N |
| 1312 | GTC TTC TCA TGC TCC GTG ATG CAT GAG GCT CTG CAC AAC CAC TAC ACA CAG AAG AGC |
| 438> | V F S C S V M H E A L H N H Y T Q K S |
| 1369 | CTC TCC CTG TCT CTG GGT tga (SEQ ID NO: 40) |
| 457> | L S L S L G * (SEQ ID NO: 41) |

The mature light chain amino acid is provided below (Kabat CDRs are underlined).

| (SEQ ID NO: 42) | |
|---|---|
| 1 | DIQMTQSPSS LSASVGDRVT ISCRASQDIS NYLNWYQQKP GKVPKLLIYF |
| 51 | TSRLRSGVPS RFSGSGSGTD YTLTISSLQP EDVATYYCQQ DRKLPWTFGQ |
| 101 | GTHLEIKRTV AAPSVFIFPP SDEQLKSGTA SVVCLLNNFY PREAKVQWKV |
| 151 | DNALQSGNSQ ESVTEQDSKD STYSLSSTLT LSKADYEKHK VYACEVTHQG |
| 201 | LSSPVTKSFN RGEC |

The light chain coding sequence including the signal peptide of native human kappa origin (GenBank CAA77299) is shown above (SEQ ID NOS: 37 and 38).

### Example 4. Expression Cassettes and Vector Maps

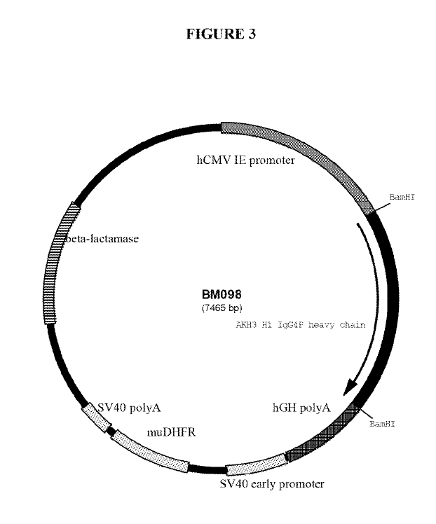

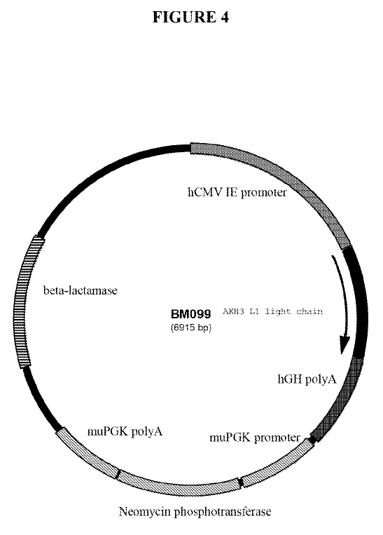

The sequences of humanized AKH3 IgG4P H1L1 were used to construct the production cell line for this antibody. The heavy and light chain expression cassettes are carried on separate plasmids. To facilitate efficient secretion and high fidelity cleavage of the signal sequence, the secretion sequence associated with the heavy chain was examined. Secretion signals were evaluated for efficiency and specificity using the SignalP prediction software. As a result of this analysis, the heavy chain signal peptide sequence, MGWSLILLFLVAVATRVLS (SEQ ID NO:43), was replaced with MRVPAQLLGLLLLWLPGARC (SEQ ID NO:44) during the vector design process. The common signal peptide for both chains is of native human kappa origin. To potentially improve expression the nucleotide sequence of the light and heavy chain including the signal sequence was recoded without changing the amino acid sequence. The new light and heavy chain DNA sequence encoding the signal peptide, variable and constant domains were synthesized de novo by DNA2.0. The heavy and light chain genes were excised from the cloning vectors and ligated into separate expression vectors, both under the control of the hCMV IE promoter. The plasmid expressing the heavy chain, BM098, contains an expression cassette for the dhfr gene which was used as a selectable and methotrexate-amplifiable marker (FIG. 3). The plasmid expressing the light chain, BM099, contains an expression cassette for the neomycin phosphotransferase gene (neo) containing the murine phosphoglycerate kinase (muPGK) early promoter and the muPGK polyadenylation sequence (FIG. 4). Plasmids BM098 and BM099 were sequenced in their entirety and found to be consistent with the electronically assembled hypothetical sequences. The key feature of plasmids BM098 and BM099 are summarized below in Table 2.

TABLE 2
Summary of BM098 and BM099 Expression Plasmids

| | | | Mature | | |
|---|---|---|---|---|---|
| | | Signal | Polypeptide | Poly- | Selectable |
| Plasmid Name | Promoters | Peptides | chain | Adenylation | Markers |
| BM098 | hCMV IE | Synthetic | Heavy Chain | hGH polyA | dhfr |
| | SV40E | signal | 463 aa | SV40 polyA | β-lactamase |
| | | peptide | | | (ampicillin) |
| | | sequence | | | |
| BM099 | hCMV IE | Synthetic | Light Chain | hGH | Neomycin |
| | muPGK | signal | 236 aa | muPGK | phosphotransferase |
| | | peptide | | | (G418) |
| | | sequence | | | β-lactamase |
| | | | | | (ampicillin) |

Abbreviations:
human cytomegalovirus immediate early (hCMV IE),
early simian virus 40 (SV40E),
murine phosphoglycerate kinase (muPGK),
human growth hormone (hGH),
neomycin phosphotransferase gene (G418 resistance),
dihydrofolate reductase gene (dhfr),
bacterial gene for resistance to ampicillin (beta-lactamase).

### Example 5. Exemplary Anti-CD40 Antibody 1

The nucleic acid (SEQ ID NO:45) and amino acid sequence 1-463 (SEQ ID NO:46) of an exemplary anti-CD40 antibody (i.e., Exemplary Anti-CD40 Antibody 1) heavy chain is provided below Amino acids 1-20 (DNA sequence shown in lower case) contain the recoded synthetic signal peptide. The mature N-terminus begins with amino acid 21 (E). The Exemplary anti-CD40 antibody 1 is an IgG4 antibody with the mutation S225P (S228P according to Kabat numbering). The mature heavy chain of Exemplary Anti-CD40 Antibody 1 consists of amino acids 21-463 of SEQ ID NO:46. The heavy chain variable region of Exemplary Anti-CD40 Antibody 1 is underlined. The S225P mutation is underlined and boldened.

| | |
|---|---|
| 1 | atg cgc gtg cct gcc caa ctt ctc gga ctt ctc ctc ctt tgg ctg cct gga ggc cga |
| 1> | M R V P A Q L L G L L L L W L P G A R |
| 58 | tgt GAA GTC CAG CTG GTG CAA AGC GGA GCC GAA GTC AAG AAG CCA GGA GCA TCG GTC |
| 20> | C E V Q L V Q S G A E V K K P G A S V |
| 115 | AAA GTG AGC TGC AAG GCT TCG GGC TAC ACT TTT ACC ACC TTC CCG ATT GAA TGG GTG |
| 39> | K V S C K A S G Y T F T T F P I E W V |
| 172 | CGC CAG GCT CCT GGT CAA GGA CTG GAG TGG ATG GGA AAC TTC CAT CCG TAC AAC GAT |
| 58> | R Q A P G Q G L E W M G N F H P Y N D |
| 229 | GAC ACC AAG TAC AAC GAG AAG TTC AAG GGC AGA GTC ACC CTC ACT GCC GAT AAG TCA |
| 77> | D T K Y N E K F K G R V T L T A D K S |
| 286 | ACC TCG ACC GCG TAC ATG GAA CTC TCA AGA CTC CGG AGC GAG GAC ACC GCC GTG TAC |
| 96> | T S T A Y M E L S R L R S E D T A V Y |
| 343 | TAT TGC GCG CGG CGG GGA AAA CTG CCG TTC GAC TCA TGG GGA CAG GGA ACT ACC GTC |
| 115> | Y C A R R G K L P F D S W G Q G T T V |
| 400 | ACC GTG TCA AGC GCG TCG ACT AAG GGC CCA TCC GTG TTT CCT CTG GCA CCC TGC TCA |
| 134> | T V S S A S T K G P S V F P L A P C S |
| 457 | CGC TCC ACC TCA GAG TCC ACT GCT GCG CTC GGG TGT CTG GTC AAA GAC TAC TTC CCT |
| 153> | R S T S E S T A A L G C L V K D Y F P |
| 514 | GAG CCA GTG ACC GTT AGC TGG AAT TCG GGC GCC CTG ACT TCT GGC GTC CAT ACT TTC |
| 172> | E P V T V S W N S G A L T S G V H T F |
| 571 | CCG GCA GTG CTC CAG TCG TCC GGC CTG TAC TCC TTG TCG TCA GTG GTG ACG GTG CCT |
| 191> | P A V L Q S S G L Y S L S S V V T V P |
| 628 | TCA AGC TCG CTG GGA ACT AAG ACC TAC ACT TGC AAC GTG GAC CAC AAG CCG TCC AAC |
| 210> | S S S L G T K T Y T C N V D H K P S N |
| 685 | ACG AAG GTC GAC AAG AGG GTC GAA TCG AAA TAC GGA CCG CCA TGC CCG CCG TGT CCA |
| 229> | T K V D K R V E S K Y G P P C P P C P |
| 742 | GCC CCC GAA TTC TTG GGA GGT CCT TCG GTT TTT CTT TTC CCG CCA AAG CCA AAG GAT |
| 248> | A P E F L G G P S V F L F P P K P K D |
| 799 | ACT CTG ATG ATC TCC CGG ACC CCC GAA GTG ACT TGC GTG GTG GTC GAT GTG AGC CAG |
| 267> | T L M I S R T P E V T C V V V D V S Q |
| 856 | GAA GAT CCA GAA GTT CAG TTT AAT TGG TAT GTG GAC GGA GTC GAG GTG CAC AAC GCC |
| 286> | E D P E V Q F N W Y V D G V E V H N A |
| 913 | AAA ACG AAG CCG AGG GAA GAA CAG TTT AAC AGC ACT TAC CGC GTG GTG TCG GTC CTC |
| 305> | K T K P R E E Q F N S T Y R V V S V L |
| 970 | ACC GTC CTG CAC CAA GAT TGG CTG AAT GGG AAA GAG TAC AAG TGC AAA GTG AGC AAC |
| 324> | T V L H Q D W L N G K E Y K C K V S N |
| 1027 | AAA GGA CTG CCG TCC TCC ATC GAA AAG ACT ATC TCG AAA GCC AAG GGG CAG CCT CGC |
| 343> | K G L P S S I E K T I S K A K G Q P R |
| 1084 | GAG CCG CAA GTG TAC ACC TTG CCA CCG TCG CAA GAA GAG ATG ACC AAG AAC CAA GTG |
| 362> | E P Q V Y T L P P S Q E E M T K N Q V |
| 1141 | TCA TTG ACT TGC CTC GTG AAG GGC TTC TAC CCG AGC GAC ATC GCG GTG GAG TGG GAG |
| 381> | S L T C L V K G F Y P S D I A V E W E |
| 1198 | TCG AAT GGA CAG CCC GAA AAT AAC TAC AAA ACC ACG CCC CCA GTG CTG GAC TCC GAT |
| 400> | S N G Q P E N N Y K T T P P V L D S D |
| 1255 | GGA TCA TTC TTC CTC TAC TCC CGC CTG ACT GTC GAC AAA TCA AGA TGG CAG GAG GGG |
| 419> | G S F F L Y S R L T V D K S R W Q E G |
| 1312 | AAC GTG TTC TCT TGC TCC GTG ATG CAT GAA GCA CTG CAC AAT CAC TAC ACC CAG AAG |
| 438> | N V F S C S V M H E A L H N H Y T Q K |
| 1369 | TCC CTC AGC CTG TCC CTG GGT TGA (SEQ ID NO: 45) |
| 457> | S L S L S L G * (SEQ ID NO: 46) |

The nucleic acid (SEQ ID NO:47) and amino acid sequence 1-236 (SEQ ID NO:38) of Exemplary Anti-CD40 Antibody 1 light chain is provided below. Amino acids 1-22 (DNA sequence shown in lower case) contain the recoded synthetic signal peptide. The mature N-terminus begins with amino acid 23 (D). The Exemplary anti-CD40 antibody 1 has a kappa chain. The mature light chain of Exemplary Anti-CD40 Antibody 1 consists of amino acids 23-236 of SEQ ID NO:38. The light chain variable region of Exemplary Anti-CD40 Antibody 1 is underlined.

| | |
|---|---|
| 1 | atg gac atg cgc gtg cct gct caa ctt ctc gga ctt ttg ctt ctc tgg ctc cct ggc |
| 1> | M D M R V P A Q L L G L L L L W L P G |
| 58 | gca aga tgt GAT ATT CAG ATG ACT CAA TCA CCA TCC TCC CTG AGC GCC AGC GTC GGA |
| 20> | A R C D I Q M T Q S P S S L S A S V G |
| 115 | GAT CGC GTG ACC ATC TCG TGC CGG GCG TCA CAA GAC ATC TCA AAC TAC CTC AAT TGG |
| 39> | D R V T I S C R A S Q D I S N Y L N W |
| 172 | TAC CAG CAG AAA CCG GGA AAA GTG CCG AAG CTG CTG ATC TAC TTC ACC TCT CGG CTG |
| 58> | Y Q Q K P G K V P K L L I Y F T S R L |
| 229 | AGA AGC GGT GTG CCG AGC CGC TTC TCC GGA TCA GGG TCA GGC ACC GAT TAC ACT CTG |
| 77> | R S G V P S R F S G S G S G T D Y T L |
| 286 | ACT ATT TCG TCC TTG CAG CCA GAG GAC GTG GCG ACC TAC TAC TGC CAAC AG GAC CGA |
| 96> | T I S S L Q P E D V A T Y Y C Q Q D R |
| 343 | AAA CTG CCA TGG ACC TTC GGA CAA GGA ACG AAG CTC GAA ATC AAG CGG ACT GTT GCC |
| 115> | K L P W T F G Q G T K L E I K R T V A |
| 400 | GCC CCC AGC GTC TTT ATC TTC CCG CCA TCC GAC GAA CAG CTG AAG TCC GGC ACG GCA |
| 134> | A P S V F I F P P S D E Q L K S G T A |
| 457 | TCG GTC GTC TGC CTG CTG AAT AAC TTC TAC CCG CGC GAA GCG AAG GTG CAA TGG AAA |
| 153> | S V V C L L N N F Y P R E A K V Q W K |
| 514 | GTC GAC AAC GCC CTC CAG AGC GGG AAT AGC CAG GAG TCG GTG ACT GAA CAG GAT TCC |
| 172> | V D N A L Q S G N S Q E S V T E Q D S |
| 571 | AAG GAC TCC ACC TAT TCG TTG TCG TCG ACC CTC ACT CTG TCA AAG GCT GAC TAC GAG |
| 191> | K D S T Y S L S S T L T L S K A D Y E |
| 628 | AAG CAC AAG GTG TAC GCC TGC GAA GTG ACT CAT CAG GGT CTG TCA TCG CCC GTG ACC |
| 210> | K H K V Y A C E V T H Q G L S S P V T |
| 685 | AAG TCG TTT AAC AGG GGC GAG TGC TGA (SEQ ID NO: 47) |
| 229> | K S F N R G E C * (SEQ ID NO: 38) |

### Example 6. Construction of Production Cell Line to Express Exemplary Anti-CD40 Antibody 1

The heavy chain (HC) and light chain (LC) coding sequences of Exemplary Anti-CD40 Antibody 1 were synthesized by DNA 2.0 to optimize the nucleotide sequence for CHO expression. These were cloned into the pv90/100 vectors as well as into uni-vectors in which either the GS or DHFR selectable marker was linked to the HC expression cassette via an IRES element. These three types of expression vectors were transfected into six different CHO hosts: DG44i, CHOS-GS host 44, CHOS-DHFR host B3 and three CHOK1-GS hosts. Transfected pools were selected as host appropriate through either glutamine or nucleoside withdrawal. Where appropriate, methotrexate (MTX) amplification strategies were also employed. Select cultures were further enriched through FACS and ClonePix to isolate high expressing cells. The most promising were subjected to limited dilution cloning coupled with imaging to insure clonality, expanded and screened in 24 deep well plate-fed batch analyses in CHOM48 medium. Based on titer and product quality analysis including aggregation levels, charge variants, fucose and mannose content, 12 DG44i and 12 CHOK1 GS Host 5 derived clones were identified as candidates for final analysis in ambr mini-bioreactors. Based on these same factors, DG44i CHO cells were selected as the production cell line to express Exemplary Anti-CD40 Antibody 1.

### Example 7. Characterization of N-Linked Carbohydrates and Modifications of Exemplary Anti-CD40 Antibody 1

The N-linked glycans of Exemplary Anti-CD40 Antibody 1 were released by treatment with peptide-N-glycosidase F (PNGase-F) and the N-linked carbohydrate distribution was determined after derivatization using anthranilamide (2AB). The modified glycans were resolved on an ACQUITY UPLC system equipped with a 1.7-μm particle, 2.1 mm×150 mm UPLC ACQUITY HILIC column (Waters) in-line with a fluorescence detector and an Orbitrap Elite-MS mass spectrometer. Oligosaccharide structure elucidation was based on the accurate mass measurements of glycans from the Obitrap, MS/MS fragment pattern, characteristic LC elution profile, and the knowledge of common mammalian N-linked glycan motifs. Simglycan software was also used for glycan identification. The distribution of N-linked glycoforms of Exemplary Anti-CD40 Antibody 1 is summarized below.

### Distribution of Glycoforms of BIIB063 Research Standard

| Glycoforms | % |
|---|---|
| Afucosylated glycan | 0.47 |
| G0 (A2F) | 87.02 |
| G1 (G1A2F) | 4.61 |
| G2 (G2A2F) | 0.41 |

| | | | |
|---|---|---|---|
| High Mannose | Man3 | 2.83 | 0.07 |
| | Man4 | | 0.57 |
| | Man5 | | 1.44 |
| | Man6 | | 0.34 |
| | Man7 | | 0.21 |
| | Man8 | | 0.20 |

| | |
|---|---|
| α-Galactosyl epitope | 0.01 |
| Acidic glycan: NGNA & NANA | 0.30 |
| Ratio of NGNA/NANA^{¥} | 0.03 |
| Galactosylation* | 2.90 |
| Sialylation^{a} | 5.25 |

*Galactosylation (%) = {Sum[Area(Gal) × Branch No. with endGal]}/{Sum[Area(Glycan^{¥}) × Branch No.]} × 100
^{¥}high mannose glycans are not included for the calculation
^{a}Sialylation (%) = {Sum[Area(Sia) × Branch No. with Sia]}/{Sum[Area(end Gal) × Branch No. with end Gal]} × 100

The detected glycoforms are mainly the asialo-, beta-galactosylated biantennary, core-fucosylated structures, G0 (87.2%), G1 (4.6%) and G2 (0.4%), afucosylated glycans (0.5%), acidic glycans (0.3%), with a relatively low percentage of high mannose glycoforms (2.6%). The amount of terminal alpha galactosylated Gal (α1-3) glycoforms is 0.01%. The ratio of N-glycolylneuraminic acid (NGNA) to N-acetylneuraminic acid (NANA) is 0.03. The Galactosylation is 2.9% and the sialylation is 5.3%.

### Modifications:

### a) Oxidation

Tryptic peptide mapping of Exemplary Anti-CD40 Antibody 1 revealed that Met-249 (5%) and Trp-158 (6%) in the heavy chain were most susceptible to oxidation. Most of the oxidation was probably generated during sample preparation.

### b) Deamidation

Analysis of the tryptic peptide map of Exemplary Anti-CD40 Antibody 1 showed that 2-2.5% each Asn-312 and Asn-381 in the heavy chain was deamidated (combined deamidation and succinimide formation). The extent of these modifications may be related to sample preparation.

### c) Glycation

Glycation is a non-enzymatic modification caused by the reaction of amino groups on proteins with glucose, a component of the culture medium. Glycation is routinely detected in proteins and levels vary widely depending on cell culture conditions. In Exemplary Anti-CD40 Antibody 1, the level of glycation, as measured by intact mass analysis of the non-reduced protein, was ˜25%. Peptide mapping analysis revealed 0.8-1.4% of the glycation on each of the residues Lys-93 and Lys-169/Lys183 of the light chain and Lys-147 and Lys-243/Lys-245 of the heavy chain.

### d) O-Linked Glycosylation

Peptide mapping analysis and MS/MS (CID and ETD) revealed that ˜0.8% of Thr-155 in the CH2 domain of the heavy chain has an O-linked HexNAc. Low-levels of this modification were observed in all cell lines and pools of clones for Exemplary Anti-CD40 Antibody 1 analyzed so far.

### e) Hydroxylysine

Peptide mapping analysis showed the Lys121 in the heavy chain can be hydroxylated (Hyl121) in Exemplary Anti-CD40 Antibody 1. The level of Hyl121 in the heavy chain was 9% in Exemplary Anti-CD40 Antibody 1. The level of hydroxylysine is clone and cell culture dependent.

### f) Other Modifications

All detected components at the ≧1%-level in tryptic map of Exemplary Anti-CD40 Antibody 1 were identified (˜1200 total). The analysis showed that ˜1.3% of the heavy chain contained a Glu10Lys mutation in the sample. The mutation is cell line dependent. The Glu10Lys mutation is most likely due to a single DNA base mutation in the Glu10 codon (GAA to AAA). No other mutations or unknown modifications at a level of ≧1% were observed in the sample.

### Example 8. FACS Direct Binding Assay

Exemplary Anti-CD40 Antibody 1 binds to CD40 on the surface of primary human B lymphocytes in whole blood with an average EC50 of 62.6 ng/mL or 0.4 nM (n=7 normal healthy donors).

### Example 9. Anti-CD40 mAbs Employed in Functional Assessments

Table 3 below lists the antibodies used in the experiments.

TABLE 3

| mAb | Isotype | Description |
|---|---|---|
| mAKH3 | Murine IgG1 | Murine anti-human CD40 |
| | | (parent of Exemplary anti- |
| | | CD40 Antibody 1) |
| chAKH3 IgG1 | Human IgG1 | Chimeric (ch) anti-human |
| | | CD40, mAKH3 V region and |
| | | human IgG1 C region |
| Aglycosyl (agly) chAKH3 | Agly Human IgG4P/IgG1 | Effectorless, chimeric anti- |
| | | human CD40, mAKH3 V |
| | | region and hybrid human C |
| | | region consisting of IgG4 |
| | | CH1-CH2 and IgG1 CH3 |
| | | domains with S228P* and |
| | | N297Q** mutations |
| Exemplary Anti-CD40 Antibody | Human IgG4P | Humanized mAKH3 V |
| 1 (hAKH3 IgG4P) | | regions and human IgG4 C |
| | | region containing S228P* |
| | | mutation |
| Agly hAKH3 IgG4P/IgG1 | Agly Human IgG4P/IgG1 | Effectorless, humanized |
| | | version of mAKH3 with V |
| | | region equivalent to |
| | | Exemplary anti-CD40 |
| | | Antibody 1, and a hybrid |
| | | human C region consisting of |
| | | IgG4 CH1-CH2 and IgG1 |
| | | CH3 domains containing |
| | | S228P* and N297Q** |
| | | mutations |
| Agly hAKH3 IgG4P | Agly Human IgG4P | Effectorless, humanized |
| | | version of mAKH3 with V |
| | | region equivalent to |
| | | Exemplary anti-CD40 |
| | | Antibody 1, and human IgG4 |
| | | C region containing S228P* |
| | | mutation |
| hAKH3 IgG4P/IgG1 | Human IgG4P/IgG1 | Humanized version of |
| | | mAKH3 with V region |
| | | equivalent to Exemplary anti- |
| | | CD40 Antibody 1, and a |
| | | hybrid human C region |
| | | consisting of IgG4 CH1-CH2 |
| | | and IgG1 CH3 domains |
| | | containing S228P* and |
| | | N297Q** mutations |
| mADH9 | Murine IgG1 | Murine anti-human CD40; |
| | | agonistic mAb |
| chADH9 IgG1 | Human IgG1 | Chimeric version of mADH9 |
| | | with mouse ADH9 V region |
| | | and human IgG1 C region; |
| | | agonistic mAb |
| chADH9 IgG4P | Human IgG4P | Chimeric version of mADH9 |
| | | with mouse ADH9 V region |
| | | and human IgG4P C region; |
| | | agonistic mAb |
| Agly chADH9 | Agly Human IgG4P/IgG1 | Effectorless, chimeric version |
| | | of mADH9 with mouse ADH9 |
| | | V region and a hybrid human |
| | | C region consisting of IgG4 |
| | | CH1-CH2 and IgG1 CH3 |
| | | domains with S228P* and |
| | | N297Q** mutations; agonistic |
| | | mAb |
| Reference anti-CD40 antibody 1 | Human IgG4P | Human anti-human CD40 V |
| IgG4P | | region and human IgG4 C |
| (“Reference Ab 1 (IgG4P)”) | | region containing S228P* |
| | | mutation |
| Reference anti-CD40 antibody 1 | Human IgG4PE | Human anti-human CD40 V |
| IgG4PE | | region and human IgG4 C |
| (“Reference Ab 1 (IgG4PE)”) | | region containing S228P* and |
| | | L325E****mutations |
| Reference anti-CD40 antibody 1 | Agly Human IgG4P/IgG1 | Human anti-human CD40 V |
| agly IgG4P/IgG1 | | region and C region consisting |
| (“Reference Ab 1 (agly | | of IgG4 CH1-CH2 and IgG1 |
| IgG4P/IgG1)”) | | CH3 domains with S228P* |
| | | and N297Q** mutations |
| Reference anti-CD40 antibody 1 | Agly Human IgG4P | Effectorless, human anti- |
| agly IgG4P | | human CD40 V region and |
| (“Reference Ab 1 (agly IgG4P)”) | | IgG4 C region with S228P* |
| | | and N297Q** mutations |
| Reference anti-CD40 antibody 1 | Human IgG4P/IgG1 | Human anti-human CD40 V |
| IgG4P/IgG1 | | region and C region consisting |
| (“Reference Ab 1 | | of IgG4 CH1-CH2 and IgG1 |
| (IgG4P/IgG1)”) | | CH3 domains |
| Reference anti-CD40 antibody 2 | Human IgG1 ala ala | Humanized, murine anti- |
| IgG1 ala ala | | human CD40 V region and |
| (“Reference Ab 2 (IgG1 ala | | human IgG1 C region |
| ala)”) | | containing L234A, L235A*** |
| | | mutations |
| Reference anti-CD40 antibody 3 | Human IgG4 | Humanized, murine anti- |
| IgG4 | | human CD40 V region and |
| (“Reference Ab 3 (IgG4)”) | | human IgG4 C region |
| Reference anti-CD40 antibody 4 | Human IgG1 | Human anti-human CD40 V |
| IgG1 | | region and human IgG1 C |
| (“Reference Ab 4 (IgG1)”) | | region |
| Reference anti-CD40 antibody 4 | Agly Human IgG1 | Human anti-human CD40 V |
| aglycosyl IgG1 | | region and human IgG1 C |
| (“Reference Ab 4 (agly IgG1)”) | | region containing N297A** |
| | | mutation |
| G28.5 | Murine IgG1 | Murine anti-human CD40; |
| | | agonistic mAb; commercially |
| | | available |

*Kabat S228P mutation stabilizes the IgG4 hinge;
**Kabat N297Q or N297A point mutation eliminates Fc glycosylation;
***Kabat IgG1 L234A, L235A point mutations reduce effector function;
****Kabat IgG4 L235E mutation reduces effector function.

### Example 10. Exemplary Anti-CD40 Antibody 1 Binding Affinity for CD40

Solution-phase affinity measurements were performed on a BIAcore 3000 instrument (BIAcore AB, Uppsala, Sweden). These studies utilized Fc-CD40 fusion proteins comprised of the human IgG1 Fc region and truncated CD40 extracellular region consisting of the first three cysteine rich domains (CRD 1-3b) from human, cynomolgus monkey, or rhesus monkey. Fc-human CD40 CRD1-3b (construct CH1261), Fc-cynoCD40 CRD 1-3b (construct pEAG3023), and Fc-rhesusCD40 CRD1-3b (construct pEAG3022), were immobilized on CM5 chips respectively, using amine-coupling chemistry in BIAcore buffer (10 mM HEPES, pH 7.2, 150 mM NaCl, 3.4 mM EDTA, 0.005% surfactant P20). Binding of Exemplary Anti-CD40 Antibody 1 Fab fragment was tested in ten cycles over a concentration range of 0 to 1.5 nM in BIAcore buffer containing 0.05% bovine serum albumin. The chips containing the immobilized Fc-CD40 constructs were regenerated with 10 mM Glycine pH 1.7 twice between each cycle. Data were analyzed with BIAevaluation 3.0 Software and were fit with 1:1 binding model. This approach allowed a true affinity to be measured without introducing an avidity component.

Exemplary Anti-CD40 Antibody 1 and mAKH3 Fab fragments bound to human CD40 with comparably high affinities (Table 4).

TABLE 4
Dissociation Constants for Anti-CD40 Fabs Binding to CD40

| | Exemplary Anti-CD40 | |
|---|---|---|
| K_{D} (M) | Antibody 1 | mAKH3 |
| Human CD40 | K_{D} ≦ 3 nM | K_{D} ≦ 3 nM |
| Cynomolgus CD40 | K_{D} ≦ 3 nM | not done |
| Rhesus CD40 | BLQ | not done |

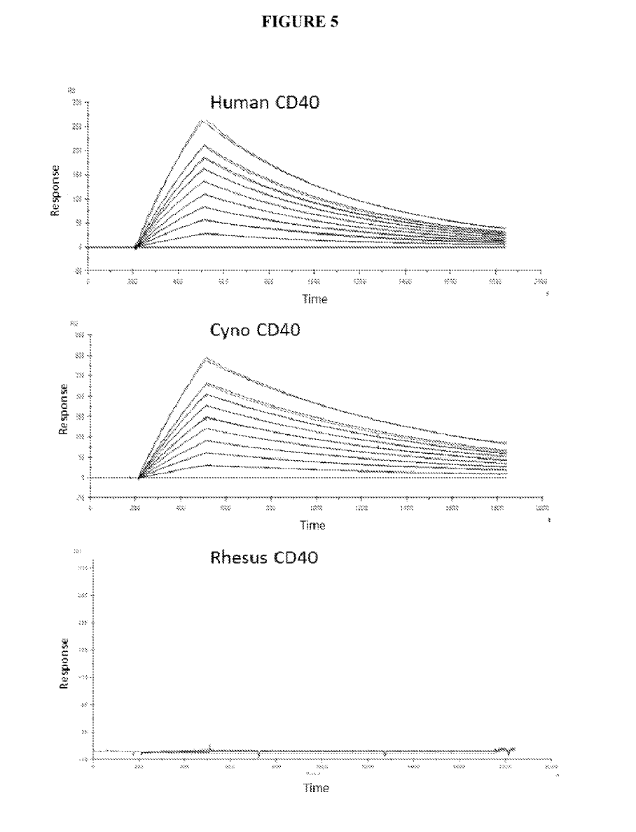

Exemplary Anti-CD40 Antibody 1 Fab fragments bound to cynomolgus monkey CD40 with the same affinity as to human CD40 (FIG. 5). In contrast, binding to rhesus CD40 was weaker and a reliable Kd value could not be determined (FIG. 5 and Table 4).

### Example 11. AKH3 Binding to Cell Surface CD40

AKH3 binding to cell surface CD40 was determined by flow cytometry on CHO cells stably transfected with full-length human, cyno, or rhesus monkey CD40, and on 293E cells transiently transfected with full-length human, rat, or mouse CD40. The mAKH3 and agly hAKH3 IgG4P/IgG1 constructs were employed, as intact mAbs or Fab fragments, and their binding detected indirectly by a secondary reagent.

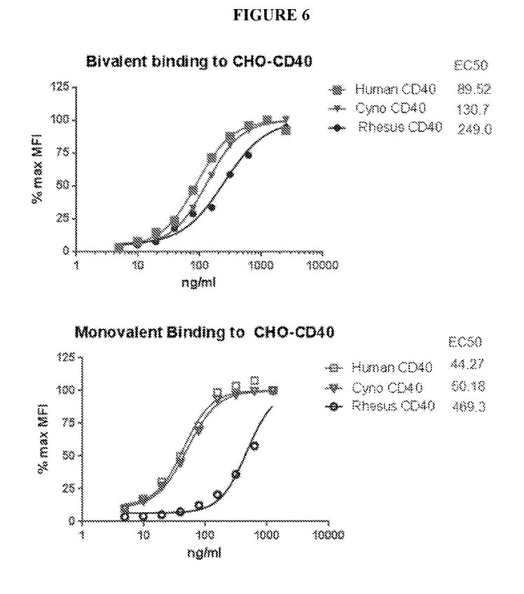

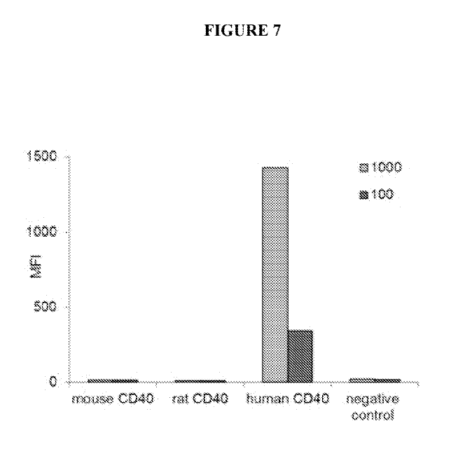

Comparable binding of AKH3 to human and cynomolgus monkey CD40 are shown in FIG. 6 for both the intact mAb (top) and Fab fragments (bottom). In contrast, weaker AKH3 binding to rhesus CD40 is indicated by the higher EC50 value of intact mAb on rhesus as compared to human and cynomolgus monkey CD40 and even more pronounced ˜10-fold increase in EC₅₀ value for the Fab fragments. In addition, mAKH3 as an intact mAb exhibited no detectable binding to rat or mouse CD40 (FIG. 7).

### Example 12. Binding to Primary Peripheral B Cells in Whole Blood

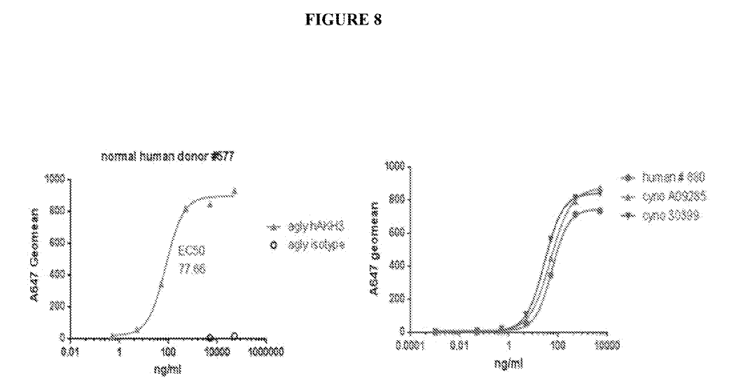

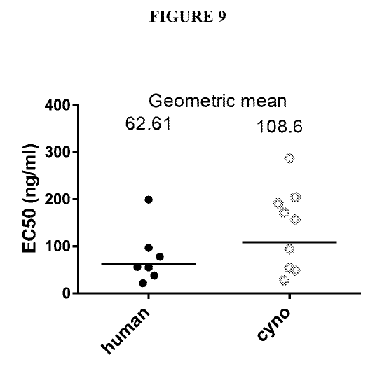

Binding to cell surface CD40 on primary B cells was measured by immunofluorescent staining of human whole blood with various concentrations of fluorochrome A647-conjugated agly hAKH3 IgG4P/IgG1, and fluorescence activated cell sorter (FACS) analysis. The staining cocktail included FITC-conjugated-anti-CD20 which was used to gate on the B lymphocytes, a key CD40-expressing cell type. A total of 7 individuals were tested. FIG. 8 (left) shows a representative agly hAKH3 IgG4P/IgG1 binding curve with an EC50 value of 0.5 nM. Likewise, agly hAKH3 IgG4P/IgG1 binding to primary B cells in whole blood was determined for 9 individual cynomolgus monkeys. Representative binding curves (FIG. 8, right) show comparable binding to human and cynomolgus monkey primary peripheral blood B cells. A summary of the binding on B cells in human and cynomolgus monkey whole blood is shown in FIG. 9.

### Example 13. Inhibition of CD40L Binding to CD40

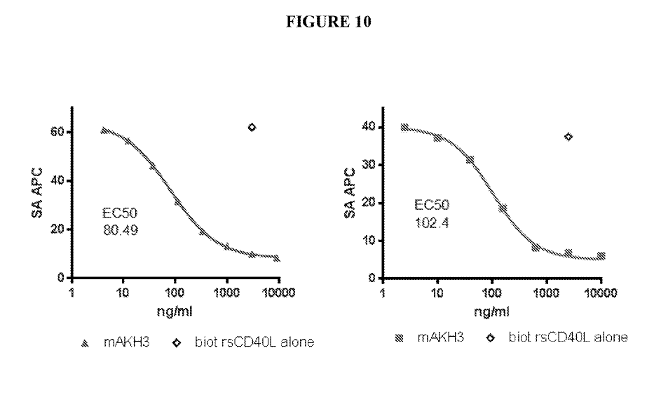

The ability of mAKH3 to inhibit CD40L binding to CD40 was measured by blocking the binding of biotinylated recombinant soluble human CD40L (rsCD40L), comprised of the CD40L ECD residues 114-261, to the RAMOS B cell line. As shown in FIG. 10, mAKH3 inhibits CD40L binding to cell surface CD40. These results demonstrate the mechanism of action whereby Exemplary Anti-CD40 Antibody 1 inhibits CD40L-induced signaling through CD40 and thereby precludes downstream functions.

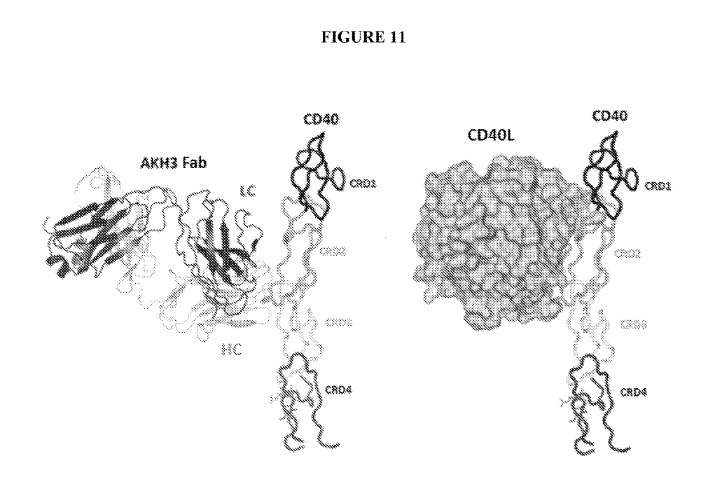

This mechanism of action is supported by structural studies. The co-crystal structure of the mAKH3 Fab with recombinant human CD40 (residues 1-170) was solved and the binding mode is shown in FIG. 11. Comparison of the mAKH3 Fab/CD40 co-crystal structure with that of the rsCD40L/CD40 co-crystal structure (An et al., J. Biol. Chem., 286:11226-35 (2011)) shows that the mAKH3 binding site on human CD40 clearly overlaps with that of CD40L.

### Example 14. Agly hAKH3 IgG4P/IgG1 Inhibition of CD40L⁺ Jurkat T Cell Stimulation of Primary B Cells

The activation of B cells by CD40L expressed on the surface of T helper cells was evaluated by co-culturing primary human B cells with the D1.1 Jurkat T cell line which constitutively expresses CD40L (CD40L⁺ Jurkat), quantifying B cell activation by the up-regulated expression of ICAM-1 (CD54) by flow cytometry. The agly hAKH3 IgG4P/IgG1 mAb has a V region identical to that of Exemplary Anti-CD40 Antibody 1. The functional potency of agly hAKH3 IgG4P/IgG1 in blocking cognate T-dependent activation was demonstrated in five normal human donors with a geometric mean IC50 value of 11 ng/mL or 0.07 nM (data not shown).

### Example 15. Exemplary Anti-CD40 Antibody 1 Inhibition of Soluble CD40L-Stimulated B Cell Activation in Whole Blood

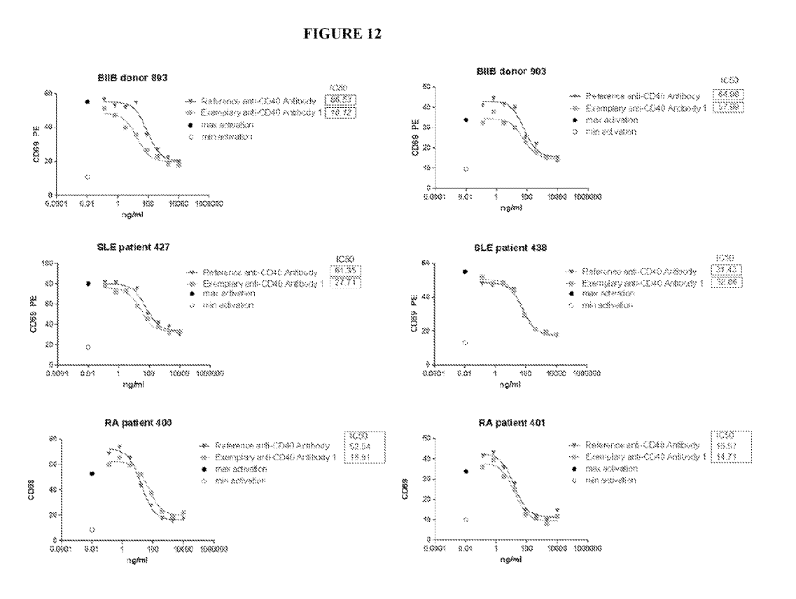

Evaluation of CD40L-dependent B cell activation in whole blood was performed using rsCD40L to stimulate CD40 signaling, since the addition of D1.1 cells to whole blood was not feasible. T helper cell activation of B cells by CD40 signaling is enhanced by co-engagement of antigen (B cell receptor signaling) or T cell-derived cytokines, notably IL-4. Thus the functional potency of Exemplary Anti-CD40 Antibody 1 in blocking CD40L-induced B cell activation was evaluated in assays of human whole blood stimulated with rsCD40L and IL-4, with B cell activation measured by FACS analysis. A concentration of rsCD40L was used that stimulated nearly maximal induction of the B cell activation markers CD69 and CD54 with similar results obtained. FIG. 12 (top) shows representative Exemplary Anti-CD40 Antibody 1 inhibition curves for rsCD40L-induced expression of the activation marker CD69 on B cells in whole blood from normal healthy donors. A total of 8 normal individuals were tested, with a geometric mean IC50 value of 51.99 ng/mL or 0.35 nM. FIG. 12 also shows that Exemplary Anti-CD40 Antibody 1 exhibits comparable functional potency to that of the Reference anti-CD40 Ab 1 (IgG4P).

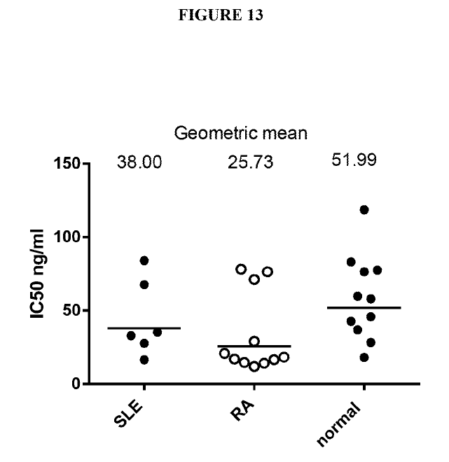

Similar experiments were conducted with whole blood from patients with SLE (n=5) and RA (n=6) and representative inhibition curves shown in FIG. 12 (middle and bottom panels). RA patients with circulating Rheumatoid factor (RF) were selected in order to investigate the functional potency of Exemplary Anti-CD40 Antibody 1 in the presence of RF which could theoretically bind to and crosslink Exemplary Anti-CD40 Antibody 1, thereby promoting agonistic activity and impeding functional potency. Representative inhibition curves (FIG. 12) show the functional potency of Exemplary Anti-CD40 Antibody 1 and its comparability with that of the Reference anti-CD40 Ab 1 (IgG4P). A summary of the functional potency of Exemplary Anti-CD40 Antibody 1 for the inhibition of rsCD40L-induced CD69 expression on B cells in human whole blood is shown in FIG. 13 indicating comparable potency between the normal, SLE and RA subjects.

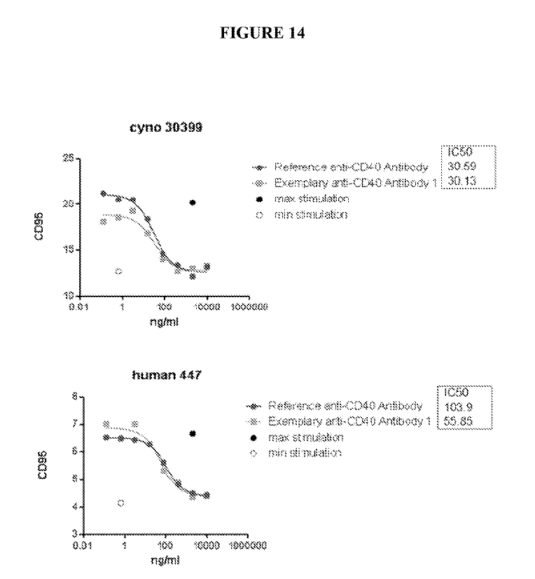

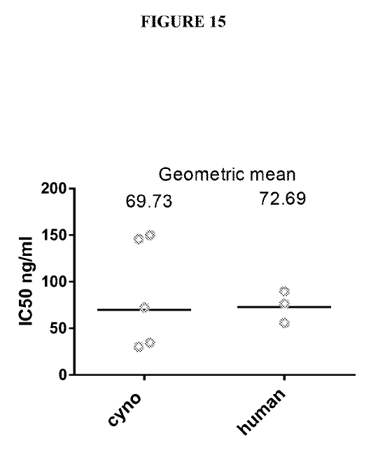

Similar experiments were conducted in assays of cynomolgus monkey whole blood stimulated with rsCD40L. The ECD of human and cynomolgus monkey CD40L are identical, thus human rsCD40L was also used for these assays. B cell activation was measured by the upregulation of the CD95 marker since a CD95-specific detection mAb was available that was highly cross-reactive with cynomolgus monkeys. FIG. 14 shows representative Exemplary Anti-CD40 Antibody 1 inhibition curves for the rsCD40L-induced B cell activation marker CD95 in whole blood cultures from a normal cynomolgus monkey as compared to a healthy human donor. FIG. 14 also shows that Exemplary Anti-CD40 Antibody 1 functional potency is comparable with that of the Reference anti-CD40 Ab 1 (IgG4P) in both cynomolgus monkey and human whole blood. A summary of the Exemplary Anti-CD40 Antibody 1 functional potency for 5 individual cynomolgus monkeys and 3 normal human donors is shown in FIG. 15. These data demonstrate the comparability of Exemplary Anti-CD40 Antibody 1 functional potency in human and cynomolgus monkey whole blood assays.

### Example 16. Assessment of Agonistic Activity in a RAMOS B Cell Line

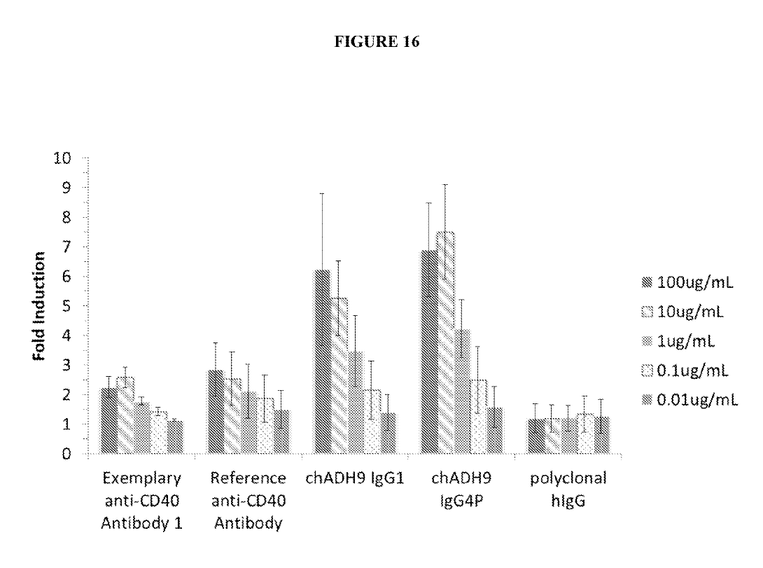

Agonistic activity was evaluated by Exemplary Anti-CD40 Antibody 1 stimulation of a human RAMOS B cell line, RAMOS Blue, harboring a stable NF-κB/AP-1-inducible SEAP (secreted embryonic alkaline phosphatase) reporter gene (Invivogen catalog# rms-sp). Soluble anti-CD40 mAbs were added at various concentrations to RAMOS Blue cell cultures and the induction of NF-κB after overnight incubation measured by alkaline phosphatase (AP) secretion in the cultured cell supernatant. The mADH9 mAb was used as a positive control for agonistic activity. FIG. 16 shows representative results for the induction of NF-κB by mADH9 but only minimal induction by Exemplary Anti-CD40 Antibody 1, and its comparability to the Reference anti-CD40 Ab 1 (IgG4P) mAb.

### Example 17. Assessment of Agonistic Activity with Primary Cells from Blood

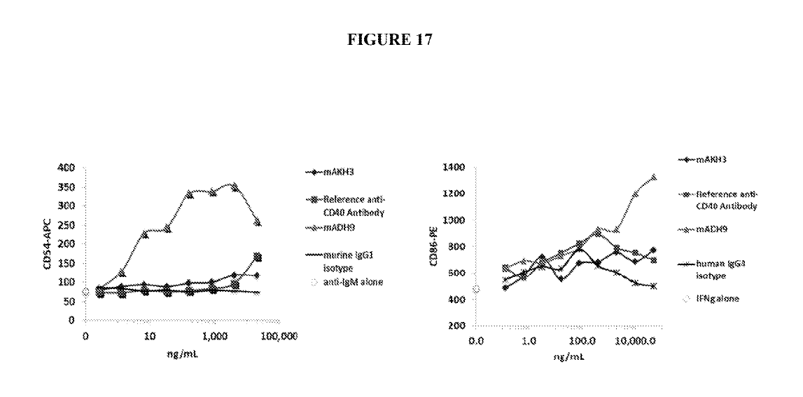

Agonistic activity was also evaluated by mAKH3 stimulation of human purified B cells and DC. Since T helper cells activate B cells by signaling through CD40 and this is enhanced by co-engagement of antigen (B cell receptor signaling) or T cell-derived cytokines, notably IL-4, anti-IgM was employed to increase the degree of B cell stimulation by an anti-CD40 agonistic positive control and thereby develop an assay sensitive to the agonistic potential of anti-CD40 mAbs. B cells and monocytes were purified from human whole blood from normal healthy donors and DC generated from the monocytes by standard methods. Soluble mAKH3 and mADH9 were added to the cultures at various concentrations and B cell and DC activation evaluated by flow cytometric measurement of the induction of activation markers, CD54 and CD86, respectively. FIG. 17 shows representative results, indicating stimulation of B cell and DC activation by mADH9 but minimal agonism by mAKH3 and its comparability to the Reference anti-CD40 Ab 1 (IgG4P).

### Example 18. Assessment of Agonistic Activity in Whole Blood

Agonistic activity was evaluated in whole blood to better model the physiologically relevant conditions in vivo in normal human donors and subjects with autoimmune disease. T helper cells activate B cells by signaling through CD40 and this is enhanced by co-engagement of antigen (B cell receptor signaling) or T cell-derived cytokines, notably IL-4. Since whole blood cultures precluded the use of anti-IgM, IL-4 was used in combination with anti-CD40 mAb to increase the degree of B cell stimulation in blood, and thereby assess potential agonistic activity.

Agonism was evaluated after overnight culturing of whole blood in the presence of IL-4 and various concentrations of soluble anti-CD40 mAbs as measured by immunofluorescent staining for the induction of B cell activation markers, CD69 and CD95. A fluorochrome-conjugated-anti-CD19 mAb was included in the staining cocktail to enable gating on the B cell population.

A total of 11 individual normal healthy human donors were assayed. In addition, 8 SLE and 10 RA patients were assayed, including 8 RA patients with circulating Rheumatoid factor (RF). Thus the agonistic activity of Exemplary Anti-CD40 Antibody 1 was investigated in the presence of RF, which could theoretically bind to and crosslink Exemplary Anti-CD40 Antibody 1, thereby promoting agonistic activity.

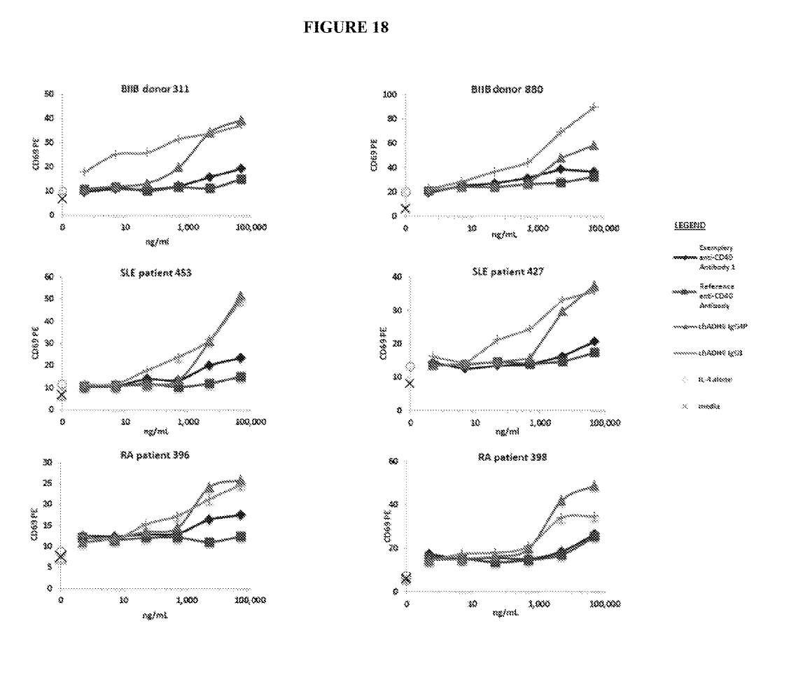

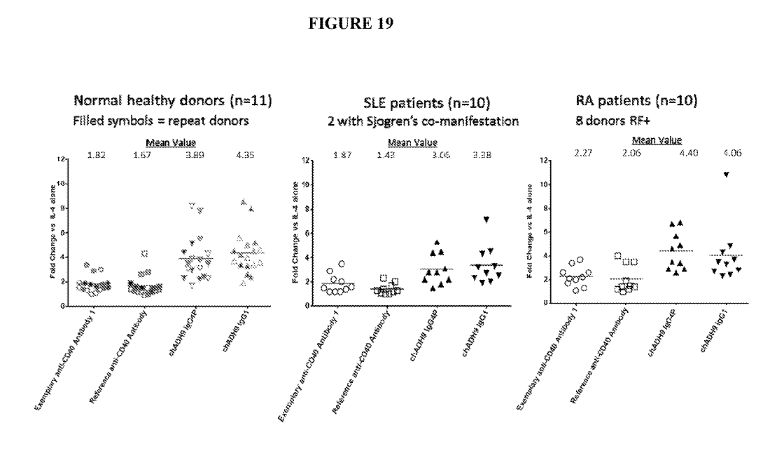

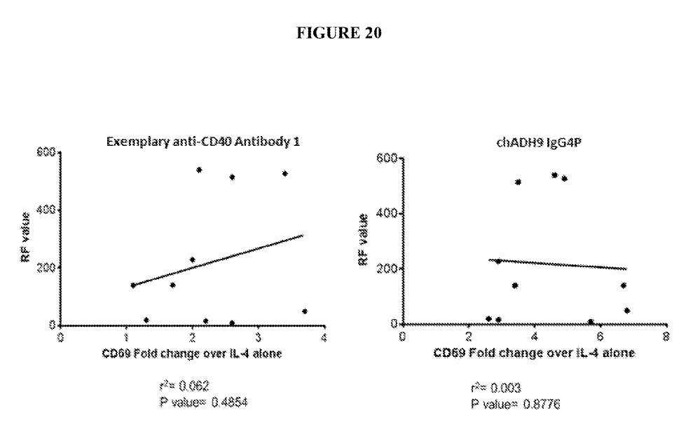

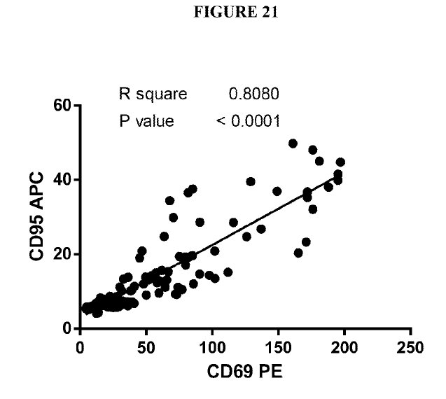

Representative results for induction of the CD69 activation marker are shown in FIG. 18, demonstrating only minimal agonism of Exemplary Anti-CD40 Antibody 1. The ADH9 mAb was used as a positive control for agonistic activity. The results for all of the donors and mAbs tested is summarized in FIG. 19, shown as the fold increase in CD69 shown for cultures with mAb and IL-4 over that of IL-4 alone. These results demonstrate that the positive control, ADH9, was consistently agonistic and Exemplary Anti-CD40 Antibody 1 minimally agonistic in whole blood cultures of normal healthy donors, SLE and RA patients. This minimally agonistic profile of Exemplary Anti-CD40 Antibody 1 is comparable to the Reference anti-CD40 Ab 1 (IgG4P). Correlation analysis further supports that the presence of RF did not increase the agonistic activity of anti-CD40 mAbs, as there is no correlation between the RF values and the corresponding results for the RA donors in the agonism assay (FIG. 20). Similar results were obtained for the CD95 marker (data not shown), which directly correlated with the CD69 activation marker results (FIG. 21).

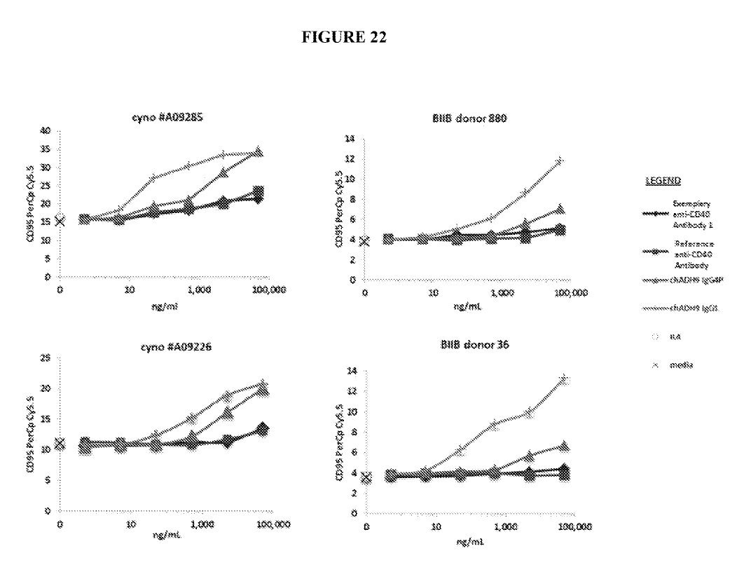

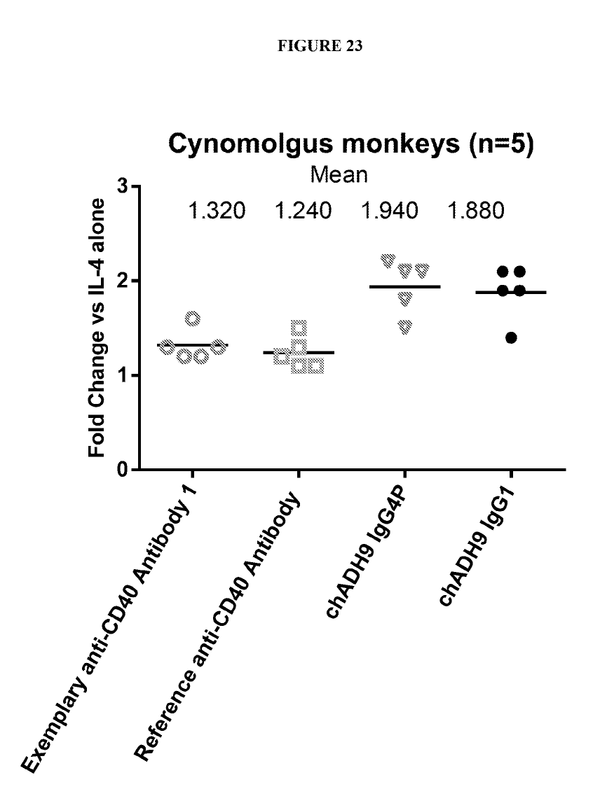

Similar experiments were conducted with cynomolgus monkey whole blood, cultured overnight with IL-4 and soluble mAbs at various concentrations followed by immunofluorescent staining for the induction of the B cell activation marker CD95, given the availability of a highly cynomolgus monkey reactive CD95-specific detection mAb. Use of CD95 as an activation marker in the cynomolgus monkey assays is also supported by the positive correlation between the results for CD69 and CD95 in the human whole blood agonism studies. A total of 5 individual monkeys were assayed in parallel with 3 individual human donors. Representative curves (FIG. 22) demonstrate that Exemplary Anti-CD40 Antibody 1 is only minimally agonistic in cynomolgus monkey whole blood cultures, in comparison to the ADH9 mAb, the positive control for agonistic activity. The summary of results (FIG. 23) further supports that Exemplary Anti-CD40 Antibody 1 is minimally agonistic in cynomolgus monkey whole blood cultures.

### Example 19. Assessment of Platelet Activation

Platelets also express the CD40 receptor. The potential for mAKH3 and a chimeric AKH3 construct with V region equivalent to that of mAKH3 to stimulate platelets was assessed by measuring the induction of the platelet activation marker P-selectin (CD62P) on platelets either in platelet-rich plasma or enriched by Sepharose gel filtration. Platelet preparations were incubated at 37° C. for 30 minutes in the presence or absence of rsCD40L, and then incubated with or without 2 μM ADP for 10 minutes at room temperature to achieve a range of sub-optimally activated states. These sub-optimal activation states predispose the platelets to respond to further agonistic signals and were included in the assay to account for donor variation in platelet activation states and sensitivities. Quiescent and sub-optimally activated platelets were then incubated with 100 μg/mL of soluble anti-CD40 mAbs for 30 minutes at 37° C.

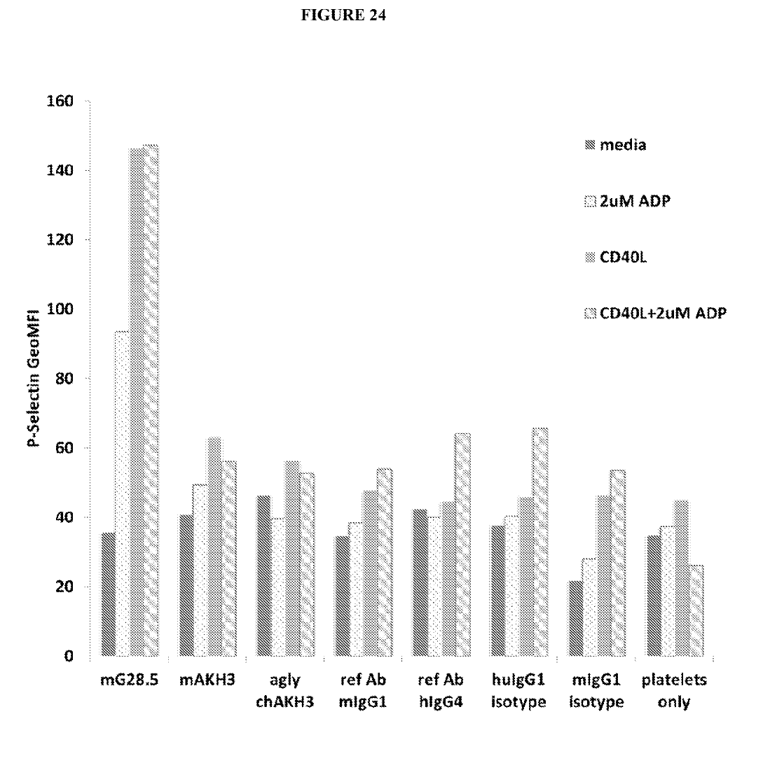

The G28.5 anti-CD40 mAb showed agonistic activity, serving as a positive control in the assay. In contrast to the G28.5 mAb, the AKH3 mAbs were not agonistic (FIG. 24). A maximal platelet activation control, defined as maximum P-selectin expression, was generated by exposing quiescent platelets to 100 μM Thrombin Receptor Activator Peptide (TRAP) for 10 minutes at room temperature.

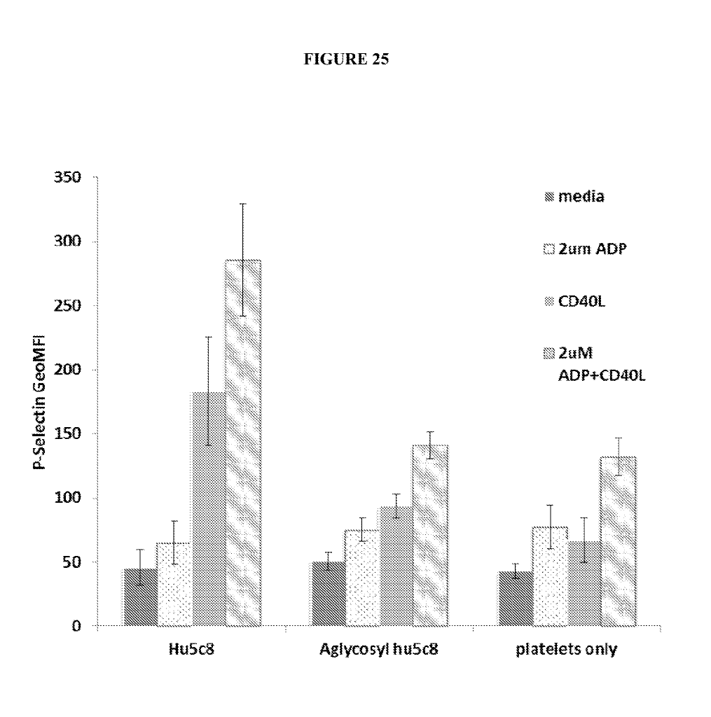

In contrast with the lack of agonism exhibited by the AKH3 mAb, antibodies to CD40L are clearly agonistic in this system (Langer, Thromb. Haemost., 93:1137-46 (2005)) and FIG. 25.

The effect of Exemplary Anti-CD40 Antibody 1 on blood clotting was also assessed in whole blood using rotational thromboelastography (ROTEM), which measures the global hemostasis in whole blood. ROTEM has demonstrated excellent correlation with efficacy of coagulation factors in bleeding models of hemophilia mice (Pan, Blood, 114:2802-2811 (2009)) and has also been reported to reflect the clinical efficacy of rFVIIa in hemophilia patients with inhibitors and acquired hemophilic patients (Kenet, Thromb. Haemostat., 103:351-359 (2010); Brophy, Haemophilia, 17:e949-957 (2011)). Increasingly ROTEM is being utilized to diagnose and treat bleeding in patients undergoing cardiac surgery or suffering from blunt trauma (Hvas, Blood Coagul. Fibrinolysis, 24:587-592 (2013); Han, Shock, 39:45-49 (2013)).

To determine whether the interaction of anti-CD40 mAb with platelets inhibits the platelet activation and result in prolonged clotting time in normal human blood, increasing doses of Exemplary Anti-CD40 Antibody 1 (0.01-100 μg/mL) were incubated with human whole blood for 1 hour at room temperature. The clotting reaction was then initiated with the addition of Ca++, and global clotting parameters including the clot initiation time (CT), clot formation time (CFT), alpha-angle and maximum clot firmness (MCF) were recorded. In comparison to untreated normal human blood that had CT of 726 sec and 682 sec in duplicate samples, Exemplary Anti-CD40 Antibody 1 showed comparable average CT in the range of 675.5 sec-760 sec irrespective of dose, in contrast to the significantly prolonged CT of 2822 sec in normal human blood treated with 3 μg/mL of anti-FVIII Ab.

In order to determine whether anti-CD40 Ab has any pro-coagulant activity, whole blood from Hemophilia A patients with prolonged baseline CT of 3740 sec to 3910 sec was utilized. The average CT of hemophilia blood pretreated with 0.01-100 μg/mL of Exemplary Anti-CD40 Antibody 1 ranged from 3095-3722 sec, indicating no significant pro-coagulant effect as compared to the CT of 1171 sec in hemophilia blood spiked in 10% of normal FVIII.

### Example 20. FcγR Binding Assay of Exemplary Anti-CD40 Antibody 1 Exemplary Anti-CD40 Antibody 1

Antibody effector function is mediated by binding of the antibody Fc region to cellular Fc gamma receptors (FcγR) and the Complement protein C1q. The Fc domain of Exemplary Anti-CD40 Antibody 1 is a fully glycosylated human IgG4, a subclass known to have reduced binding to FcγR as compared to IgG1 and devoid of interaction with Complement due to its unique CH2 sequence. These Fc functions for Exemplary Anti-CD40 Antibody 1 were evaluated.

In order to confirm the expected reduced binding profile of Exemplary Anti-CD40 Antibody 1 to human FcγR, relative binding affinities were measured using Amplified Luminescent Proximity Homogeneous Assay (ALPHAscreen) technology from Perkin Elmer. With this technology, binding pairs are immobilized onto “donor” and “acceptor” beads. Upon laser excitation, donor beads release singlet oxygen that reacts with acceptor beads in close proximity (≦200 nm) generating a cascade of events that ultimately results in fluorescence emission at 520-620 nm.

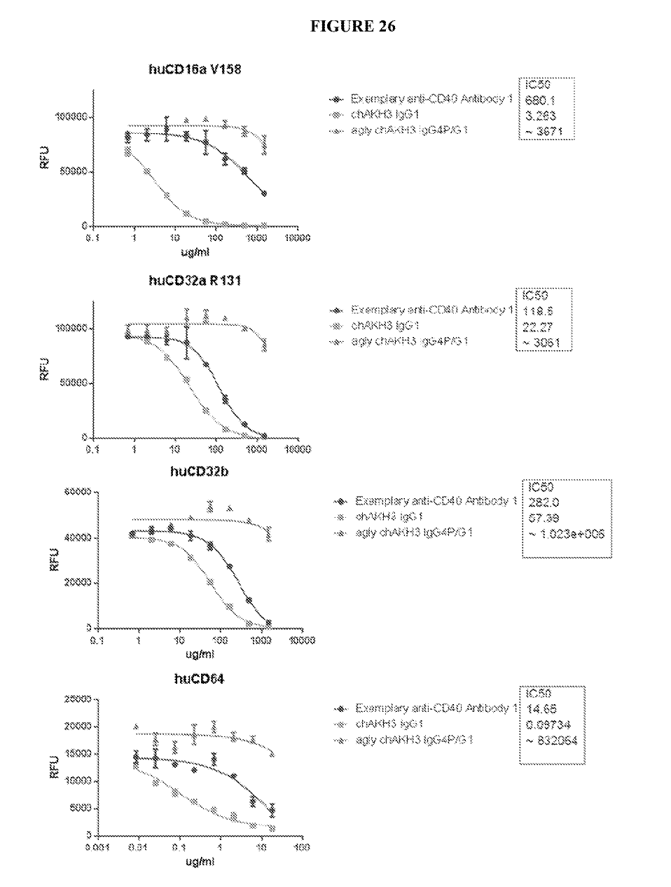

The chimeric AKH3 antibody constructs with the human IgG1 and aglycosyl IgG4P/G1 Fc regions were included in the FcγR and C1q assays as Fc competent and Fc-effectorless comparators, respectively. The assay was performed in a competitive format in which binding of test antibodies to FcγR disrupts the interaction of biotinylated IgG1 and FcγR-GST fusion protein immobilized on Streptavidin donor beads and anti-GST acceptor beads respectively. The plates were read using an Envision plate reader (Perkin Elmer) and the resulting relative fluorescence units (RFU) were plotted versus the concentration of test IgG as shown in FIG. 26. Exemplary Anti-CD40 Antibody 1 exhibits reduced binding as compared to a WT IgG1, ˜200-fold for CD16a, ˜5-fold for CD32a and CD32b, and ˜150-fold for CD64.

### Example 21. C1q Binding Assay of Exemplary Anti-CD40 Antibody 1

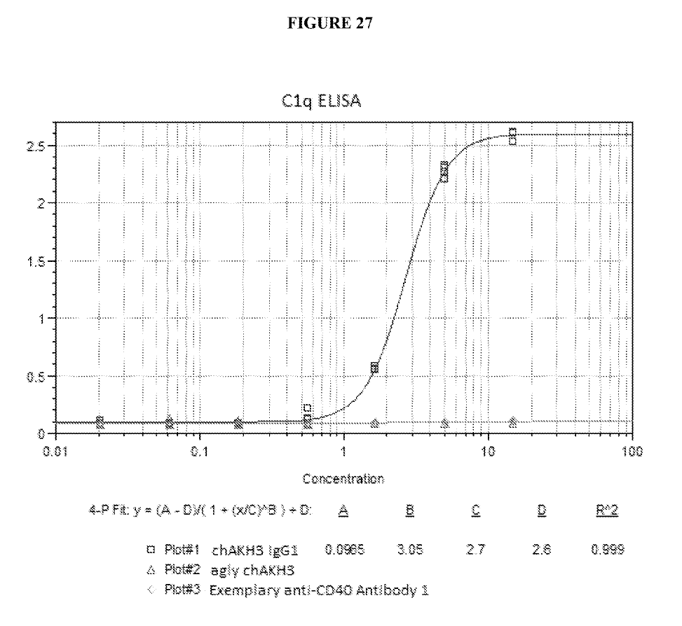

It was also determined that Exemplary Anti-CD40 Antibody 1 is not capable of activating complement by testing its binding to C1q. The assay (adapted from Idusogie et al., J. Immunol., 164:4178-84 (2000)) was conducted in an ELISA format where titrations of the test antibodies are coated in the wells and binding of human C1q is detected with chicken IgY anti-human C1q (custom reagent from Ayes Labs) followed by a donkey F(ab′) 2 anti-chicken IgY HRP conjugate. FIG. 27 shows that while chAKH3 IgG1 is capable of binding C1q, Exemplary Anti-CD40 Antibody 1, and aglycosyl hAKH3 are essentially devoid of C1q binding.

### Example 22. Antibody-Dependent Cell-Mediated Cytotoxicity

The ability of Exemplary Anti-CD40 Antibody 1 to mediate depletion was assessed in vivo in cynomolgus monkeys. There was no evidence of cell depletion, as evidenced by no change in absolute B cell numbers in the circulation, and no significant changes in total lymphocyte or white blood cell counts.

### Example 23. Effect of Removal of N-Linked Glycosylation Site

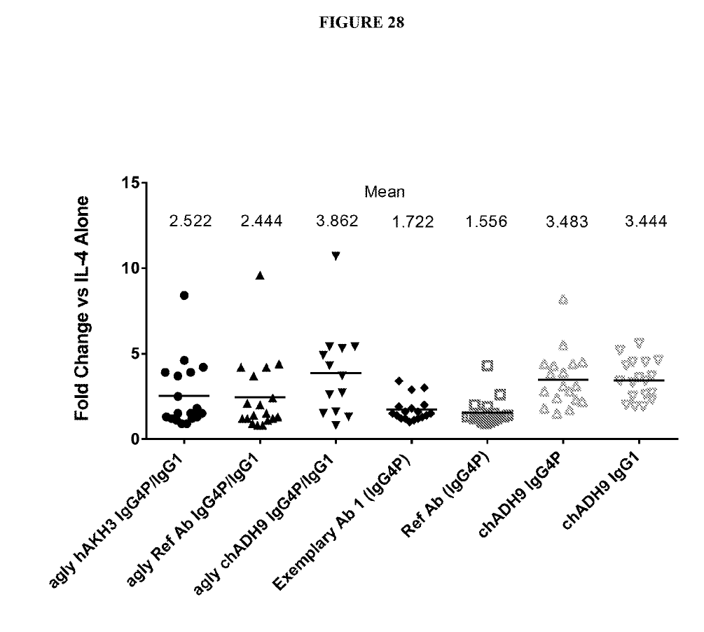

Exemplary Anti-CD40 Antibody 1 (hAKH3 IgG4P) and the Fc-effectorless construct aglycosyl hAKH3 IgG4P/IgG1 (agly hAKH3 IgG4P/IgG1) were employed to investigate the effect of glycosylation on activity. Exemplary Anti-CD40 Antibody 1 and agly hAKH3 IgG4P/IgG1 exhibited identical binding properties and potency profiles, however they differed in their agonistic profile, with agly hAKH3 IgG4P/IgG1 being more agonistic. Matched sets of antibodies constructs were produced to evaluate the agonistic potential of hAKH3, Reference anti-CD40 antibody 1, and ADH9 on IgG4P versus agly IgG4P/IgG1 scaffolds. A fully Fc-competent form of the agonistic antibody, ADH9 (chADH9 IgG1) was included as a positive control. While ADH9 retained its agonistic profile regardless of the scaffold (IgG4P, IgG1, agly IgG4P/G1), the agly IgG4P/IgG1 forms of hAKH3 and the Reference anti-CD40 antibody 1 were consistently more agonistic than IgG4P forms in whole blood assays using nine normal human donors and eight SLE donors as shown in FIG. 28.

### Example 24. Inhibition of the Humoral Immune Response to Tetanus Toxoid (TT)

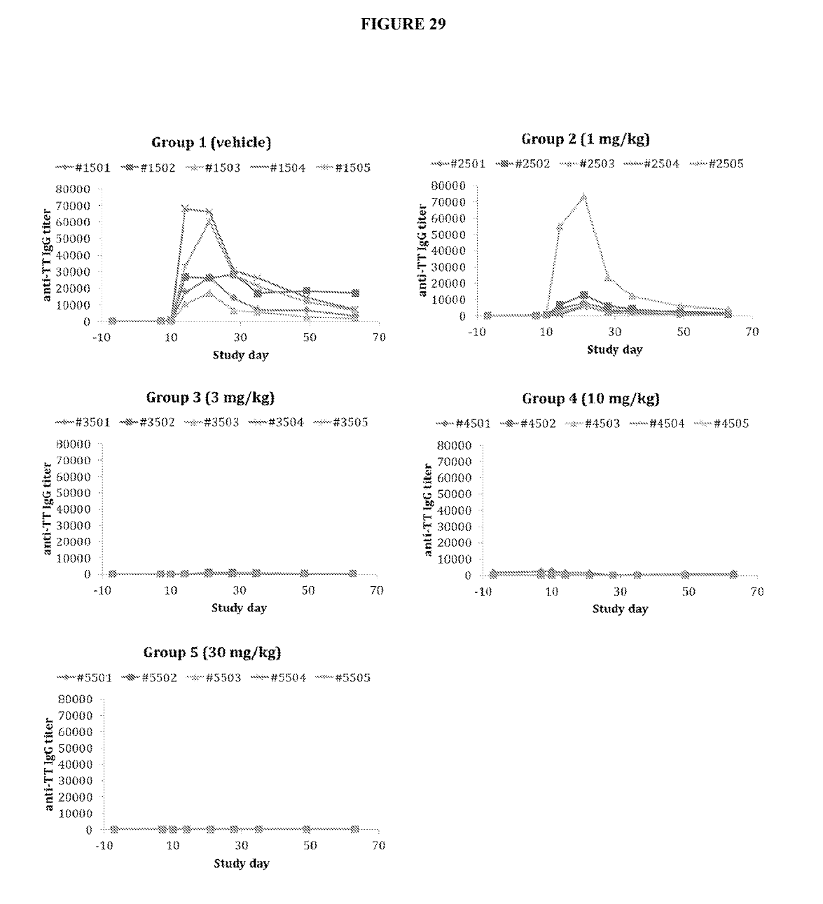

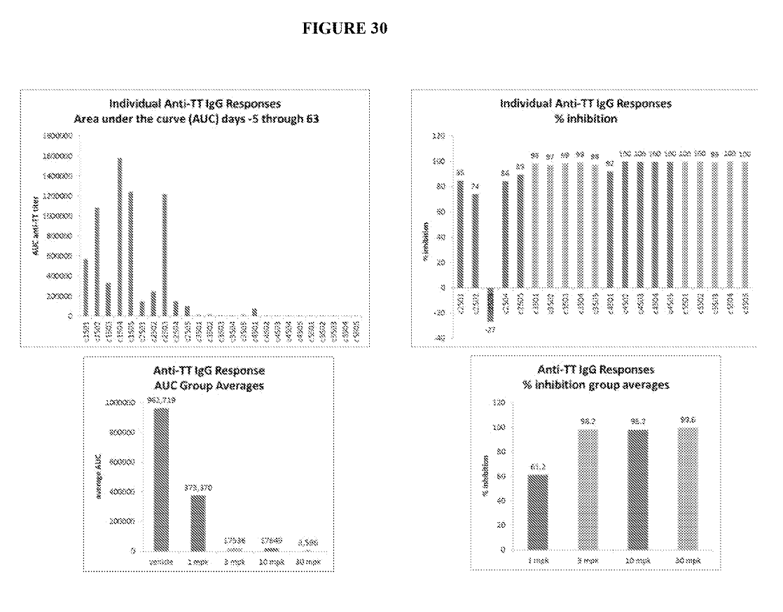

Cynomolgus monkeys received a single intravenous (iv) injection of vehicle or Exemplary Anti-CD40 Antibody 1 at 4 dose levels: 1, 3, 10, and 30 mg/kg, with n=5 cynomolgus monkeys/dose group. Exemplary Anti-CD40 Antibody 1 was injected on day 0, and TT was administered by intramuscular (IM) route 4 hours post-dose. Anti-TT antibody titers were measured in a standard ELISA format using immobilized TT (Reagent Proteins #PFE-103) to capture the Ag-specific antibodies followed by detection with anti-monkey IgG HRP (Rockland #617-103-012) and development with TMB substrate. The plates were read and data analyzed using a Spectramax plate Reader and SoftMax Pro software from Molecular Devices (Sunnydale, Calif.). The cynomolgus monkey serum was serially diluted from 1:50 to 1: 109350 and the resulting optical densities (OD) at 450 nm were plotted against the dilution factor. The titer was determined by interpolating the reciprocal dilution that resulted in 0.25 OD units over the plate background value. The resulting titers are graphically represented in FIG. 29. The area under the curve (AUC) was calculated using GraphPad Prism and this data was utilized to calculate the percent inhibition as compared to the average AUC for the vehicle treated group (FIG. 30). There was a dose dependent inhibition of anti-TT titers observed in the Exemplary Anti-CD40 Antibody 1 treated groups as compared to the vehicle treated group. Partial inhibition of 61% was observed at a dose of 1 mg/kg and nearly complete (>98%) inhibition of anti-TT was observed at doses >3 mg/kg. Of note, there was a single animal treated with 1 mg/kg (#2503) in which the anti-TT response was not inhibited and this animal is included in the group average shown in FIG. 30. The percent inhibition in the remaining four animals in Group 2 ranged from 74-89%. Based on historical experience with this in vivo TT model, Exemplary Anti-CD40 Antibody 1 is more efficacious than molecules that target CD40L.

### Example 25. Exemplary Anti-CD40 Antibody 1 Exposure in Cynomolgus Monkeys

Exemplary Anti-CD40 Antibody 1 exhibited dose-dependent clearance and half-life of Exemplary Anti-CD40 Antibody 1 over the 1-30 mg/kg dose range. As dose increased, clearance decreased and half-life increased consistent with a target-mediated drug disposition (TMDD) profile. The clearance mechanism of Exemplary Anti-CD40 Antibody 1 consists of both first order and target mediated pathways. Clearance ranged from 7.4 to 39 mL/day/kg, and half-life ranging from 2.2 to 7.8 days over the 1-30 mg/kg dose range. The volume of distribution was consistent across four dose levels (83-100 mL/kg). The small volume of distribution suggests that Exemplary Anti-CD40 Antibody 1 was primarily restricted to the extracellular space.

### Example 26. CD40 Receptor Occupancy

A flow cytometric assay was developed to evaluate total and unoccupied CD40 on the surface of cynomolgus monkey B cells in whole blood. For this assay, 100 μl of whole blood was collected in sodium heparin tubes and incubated with a multicolor immunofluorescent staining cocktail, including CD45 and CD20 antibodies, used to gate on B cells. CD40 target engagement in cynomolgus monkey whole blood by Exemplary Anti-CD40 Antibody 1 was measured using Alexa647-conjugated Exemplary Anti-CD40 Antibody 1. Total CD40 cell surface levels in cynomolgus monkey whole blood was measured using Alexa488-conjugated anti-CD40 mAb, which binds to a CD40 epitope distinct from that of Exemplary Anti-CD40 Antibody 1. Background staining on B cells was measured using a human IgG4-Alexa647 labeled isotype control antibody instead of Alexa647-Exemplary Anti-CD40 Antibody 1. All immunofluorescent staining was done in the dark, on ice. All data was acquired using a BD FACS Canto II machine, and analyzed using FlowJo and GraphPad Prism software.

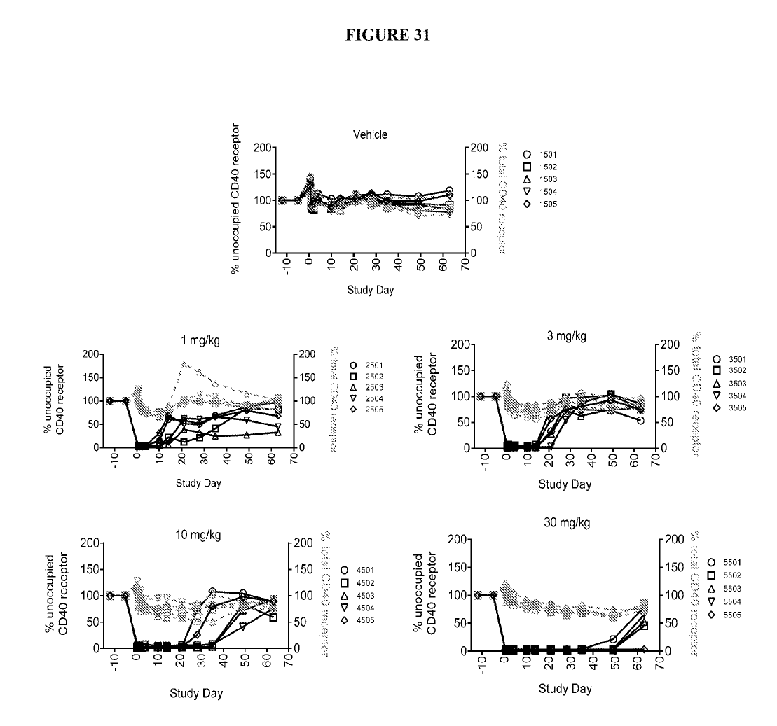

For each cynomolgus monkey in the PK/PD study, maximal CD40 density on the B cell surface was established in 2 pre-bleed samples. The average geometric mean fluorescence intensity of these time points was considered the baseline (or 100% available CD40). As shown in FIG. 31, Exemplary Anti-CD40 Antibody 1 administration resulted in saturation of the B lymphocyte CD40 receptor in all dose groups. Whereas Alexa647-Exemplary Anti-CD40 Antibody 1 staining was maintained in the vehicle-treated group, it was >95% reduced post-administration of Exemplary Anti-CD40 Antibody 1 for all dose levels for at least 4 days. Exemplary Anti-CD40 Antibody 1 levels in whole blood declined over time, in a dose-dependent manner, as indicated by recovery of Alexa647-Exemplary Anti-CD40 Antibody 1 staining Rather unexpectedly, the unoccupied CD40 levels in the 1 mg/kg group did not return to baseline. It is hypothesized that this is due to the existence of anti-drug antibodies (ADA) that neutralized the ability of the Exemplary Anti-CD40 Antibody 1-conjugate to bind to CD40 in the whole blood.

Total CD40 receptor levels, as determined by the binding of the Alexa488-labeled antibody to the CD40 receptor on an epitope distinct from Exemplary Anti-CD40 Antibody 1, remained relatively stable throughout the 63 day study, with only ˜25% decline from baseline levels (FIG. 31). It is hypothesized that decline is due to either in vivo internalization, shedding of the CD40 receptor, or steric hindrance with the unlabeled drug.

### Example 27. B Cell Frequency

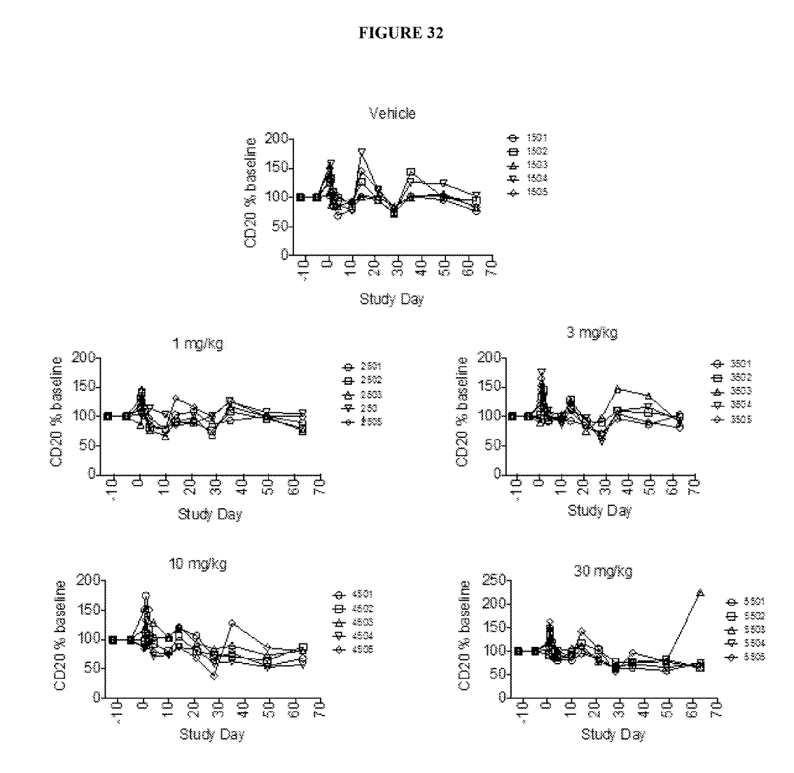

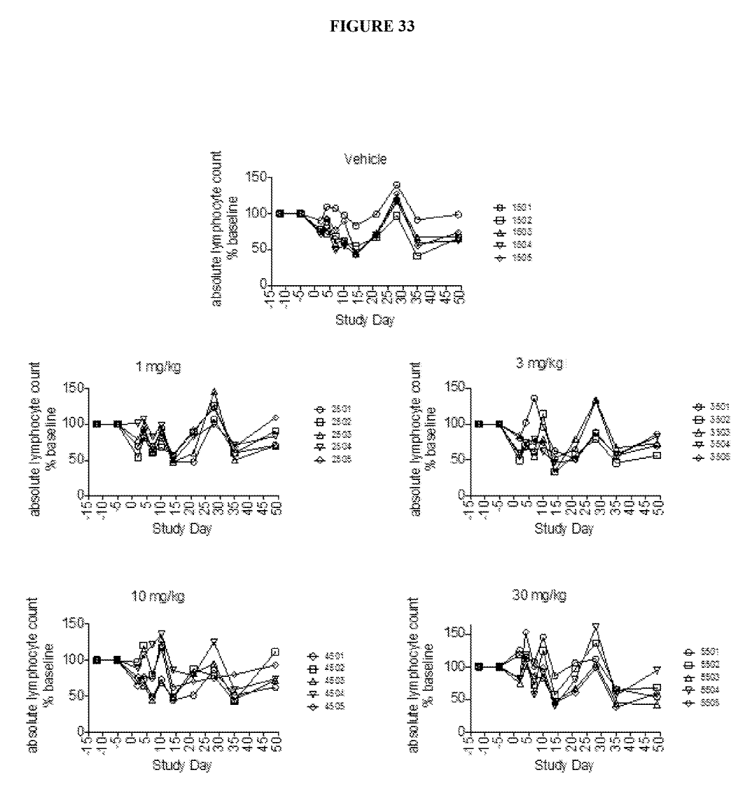

To evaluate potential cell depleting activity in vivo, B cell frequency was assessed by flow cytometry. There were transient changes in the percentage of circulating B cells in all Exemplary Anti-CD40 Antibody 1 cohorts, comparable to those in the vehicle-treated group. However, there was a sustained downward trend in the total B cell percentage in the 2 highest dose groups (FIG. 32). Likewise, fluctuations in total lymphocyte counts relative to baseline were observed. These fluctuations were similar between the Exemplary Anti-CD40 Antibody 1 and vehicle-treated groups (FIG. 33).

### Example 28. B Cell Activation Markers

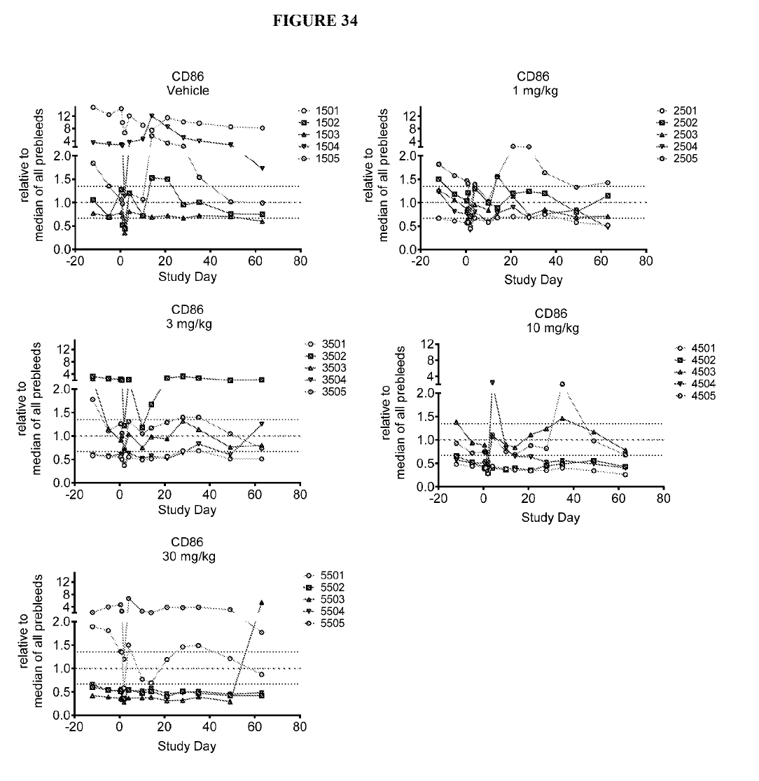

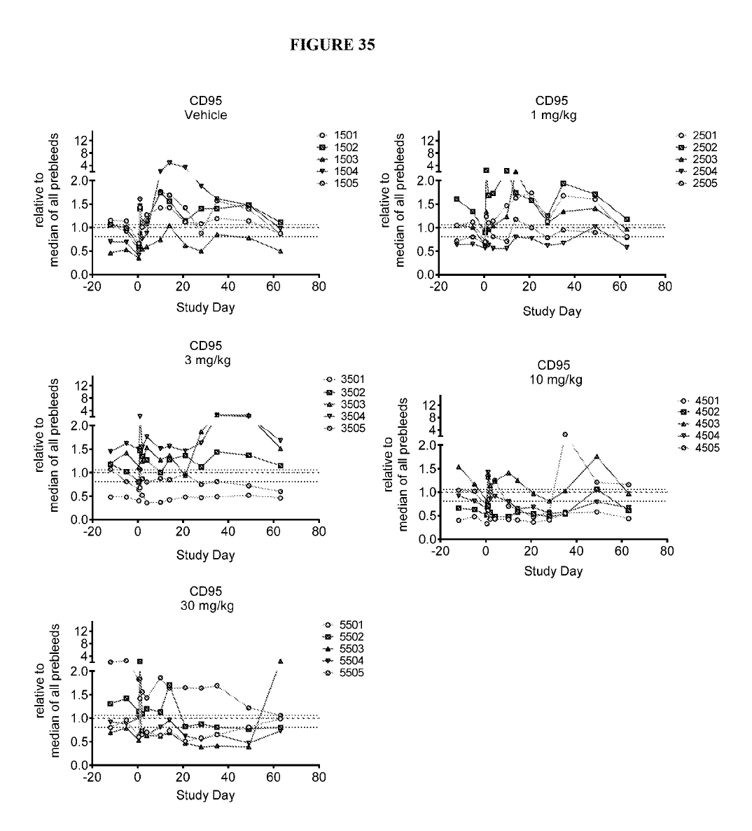

To evaluate potential agonist activity of Exemplary Anti-CD40 Antibody 1 in vivo, the levels of the B cell activation markers CD86 and CD95 were measured by flow cytometry using fluorochrome conjugated antibodies specific for these markers (PE-Cy7-CD86 and PE-CD95). CD40 mediated upregulation of these molecules on the surface of B lymphocytes has previously been reported. (Khalil and Vonderheide, Update Cancer Ther., 2:61-65 (2007)). In all dose groups, fluctuations in CD86 (FIG. 34) or CD95 (FIG. 35) expression on B cells were transient, and comparable to changes seen in the vehicle-dosed group, suggesting an absence of agonist activity in vivo. For these analyses, the median value and 95% confidence interval (for the median) for the levels of CD86 and CD95 on the B cell surface (geo mean) were calculated using all the pre-dosing timepoints (2 timepoints/monkey for 25 monkeys). On the graphs, the level of CD86 or CD95 is expressed relative to the median value (median value is set to 1).

### Example 29. Serum Cytokines

Signaling through CD40 induces the production of inflammatory cytokines, such as IL-6, TNF, and IL-12 (Vonderheide et al., J. Clin. Oncol., 25:876-83 (2007)). To assess Exemplary Anti-CD40 Antibody 1-induced changes in the serum levels of these and other cytokines, a custom Luminex magnetic bead multiplex panel (Life Technologies) was used to analyze 16 cytokines and chemokines, namely IL-1β, IL-IRA, IL-2, IL-4, IL-6, IL-8, IL-12, IL-17, TNFα, IFNγ, MIP-1α, MIP-1β, MCP-1, VEGF, Eotaxin, and RANTES. Frozen serum from all cynomolgus monkeys was stored at −80° C. For each individual monkey, the serum from various time points was assayed on a single 96-well plate, in addition to a 10-point standard curve and serum-specific positive and negative controls. Serum was run undiluted and the assay performed according to manufacturer's protocol.

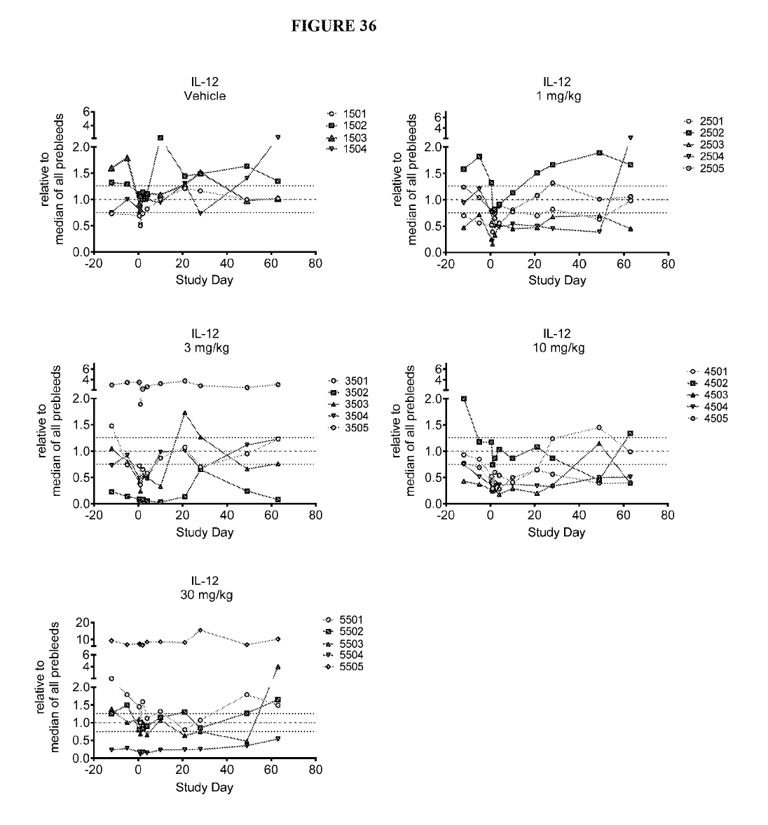

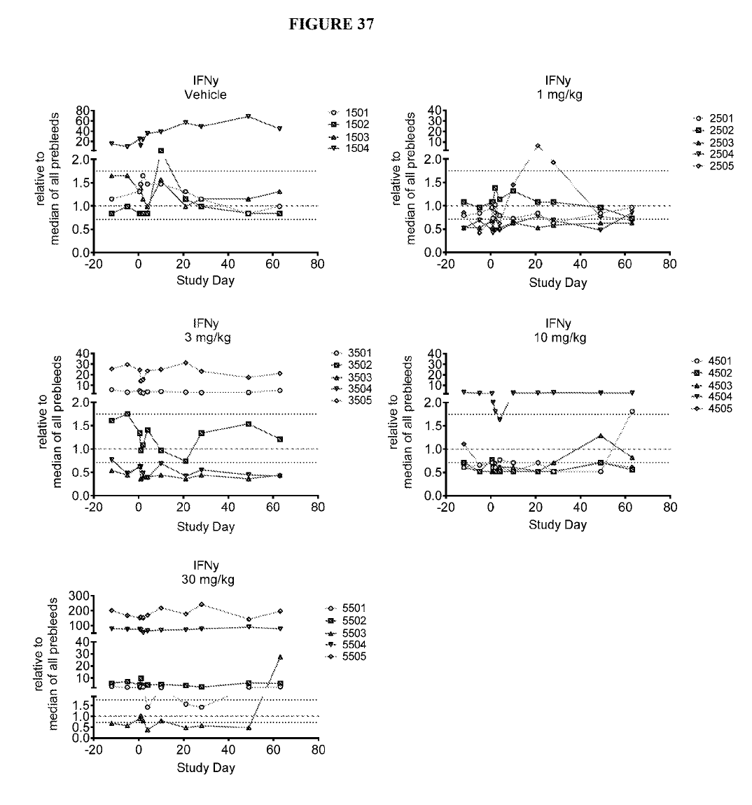

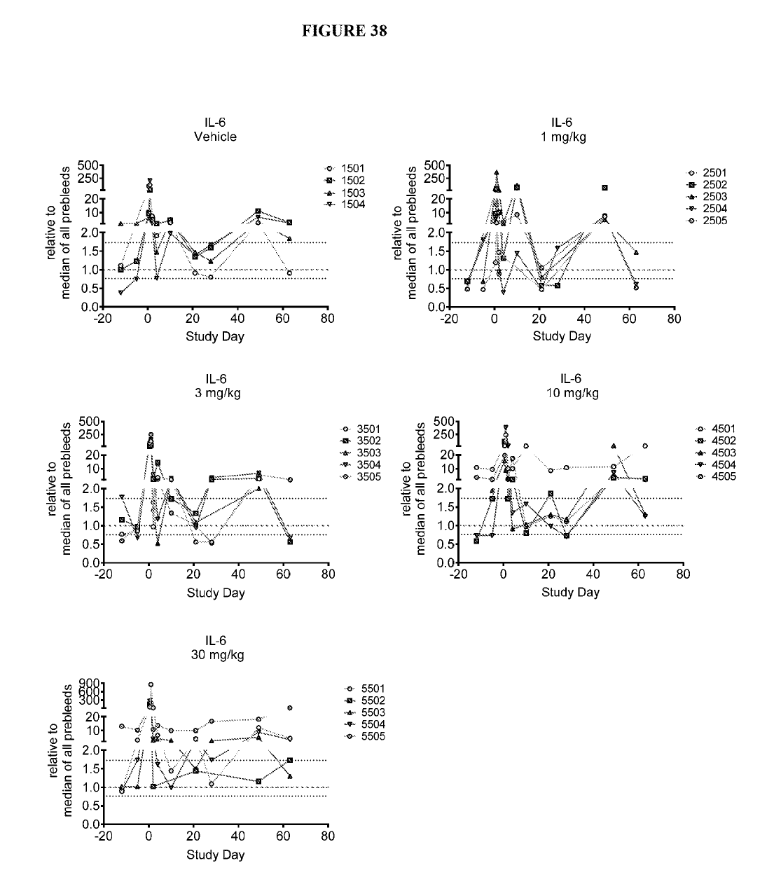

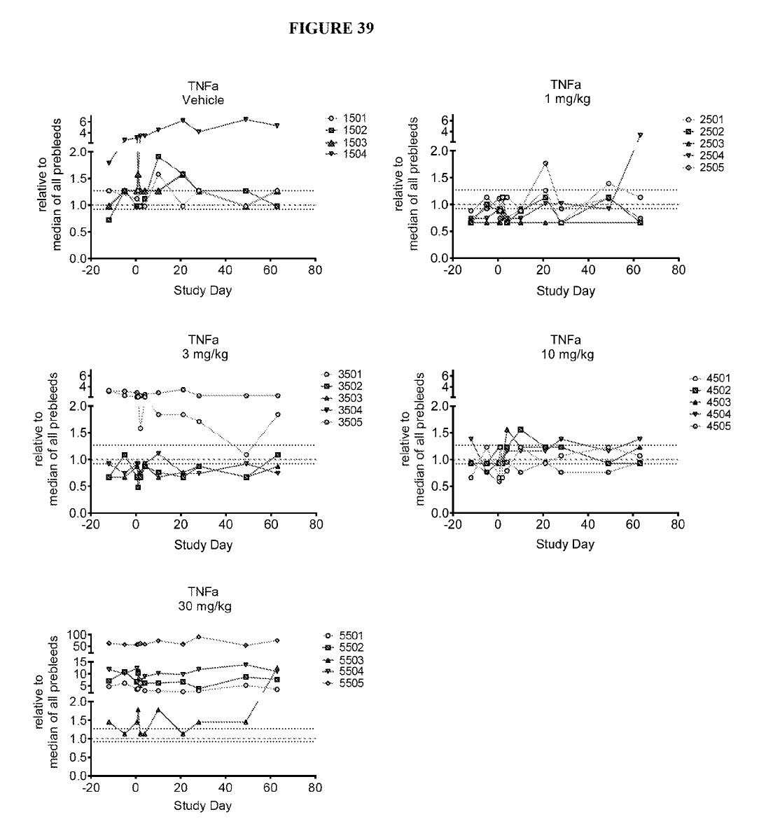

The results for IL-12 (FIG. 36), IFNγ (FIG. 37), IL-6 (FIG. 38) and TNFα (FIG. 39) are shown. For these analyses, the median value and 95% confidence interval were calculated using all the pre-dosing timepoints (2 timepoints/monkey for 25 monkeys). On the graphs, the level of analyte is expressed relative to the median (median value is set to 1). For all these cytokines, there were transient changes observed, including in the vehicle control group, with no apparent dose-dependent effect of Exemplary Anti-CD40 Antibody 1. IL-17, VEGF and IL-4 were below limits of detection (not shown).

### Example 30. Clinical Pathology Readouts

Standard clinical pathology panels were evaluated (hematology, clinical chemistry and coagulation), as well as additional parameters to interrogate potential changes in platelets and other readouts (C-reactive protein, amylase and lipase, and D-dimer analysis) that could indicate Exemplary Anti-CD40 Antibody 1 agonist signaling through CD40. There were no apparent changes in these readouts as a result of Exemplary Anti-CD40 Antibody 1 administration.

### Example 31. Exposure-Efficacy Relationships

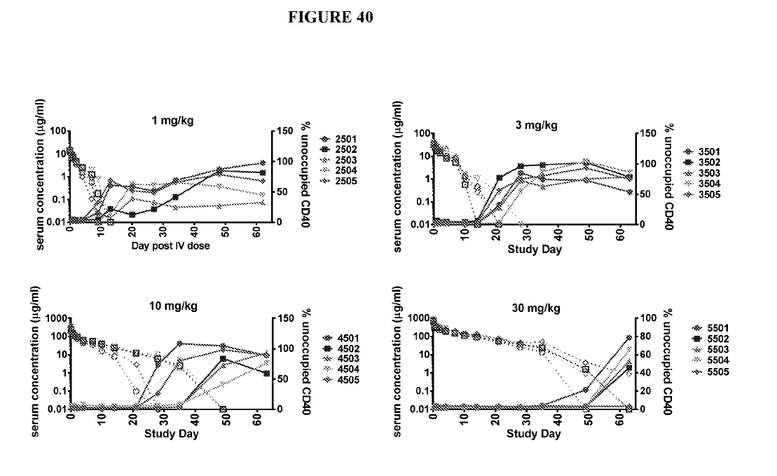

Exemplary Anti-CD40 Antibody 1 occupancy of the CD40 receptor correlated with exposure to and Exemplary Anti-CD40 Antibody 1. The Exemplary Anti-CD40 Antibody 1 serum concentration that resulted in 50% CD40 receptor occupancy (EC₅₀) was 0.28±0.27 μg/mL. The EC₅₀ for individual cynomolgus monkeys ranged from 0.05 to 0.89 μg/mL (FIG. 40).

### Example 32. Target Selectivity

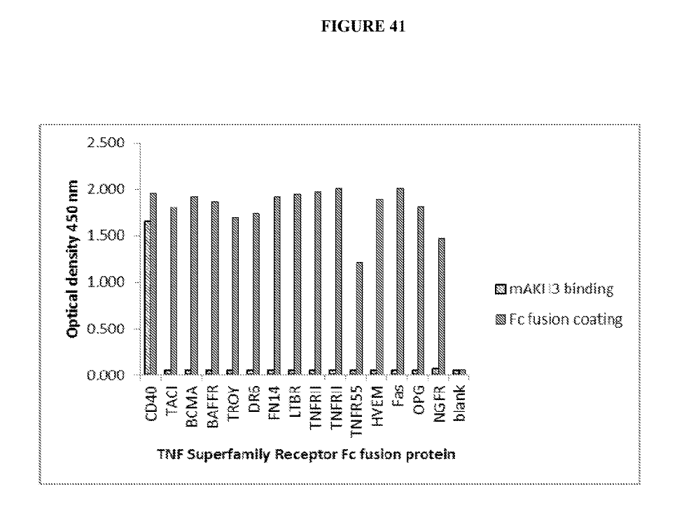

Reactivity of mAKH3 was assayed against an array of other TNF superfamily receptors by standard ELISA method. As shown in FIG. 41, mAKH3 specifically bound to human CD40 and showed no detectable interaction with the other fourteen human TNF superfamily receptors tested.

### Example 33. Species Cross-Reactivity

The sequence identity among CD40 proteins of different species is shown in Table 5 below. Percent identity among pair-wise comparisons is indicated.

Lack of cross-reactivity of Exemplary Anti-CD40 Antibody 1 to rodent CD40 was shown by lack of binding to murine or rat CD40 expressed on the surface of 293E transfected cells. Weak cross-reactivity of Exemplary Anti-CD40 Antibody 1 to rhesus CD40 was shown by BIAcore and flow cytometry measurements. The cross-reactivity of Exemplary Anti-CD40 Antibody 1 to human and cynomolgus monkey CD40 was shown by BIAcore and cell surface binding measurements and by inhibition of rsCD40L-induced B cell activation in whole blood.

The sequence alignments of human, cynomolgus monkey and rhesus monkey CD40 extracellular domains (ECDs)—the four CD40 cysteine rich domains (CRD1 (cyno: SEQ ID NO:48; rhesus: SEQ ID NO:48; human: SEQ ID NO:48); CRD2; (cyno: SEQ ID NO:49; rhesus: SEQ ID NO:50; human: SEQ ID NO:51); CRD 3 (cyno: SEQ ID NO:52; rhesus: SEQ ID NO:53; human: SEQ ID NO:54); and CRD 4 (cyno: SEQ ID NO:55; rhesus: SEQ ID NO:55; human: SEQ ID NO:56))—are shown below with the amino acid differences from human italicized and the AKH3 contact residues on human CD40 are underlined.

### CD40 Cysteine Rich Domains (CRDs)

CRD1

| (SEQ ID NO: 48) | |
|---|---|
| CYNO | ACREKQYLINSQCCSLCQPGQKLVSDCTEFTETECL |
| (SEQ ID NO: 48) | |
| RHESUS | ACREKQYLINSQCCSLCQPGQKLVSDCTEFTETECL |
| (SEQ ID NO: 48) | |
| HUMAN | ACREKQYLINSQCCSLCQPGQKLVSDCTEFTETECL |

CRD2

(SEQ ID NO: 49)

| | |
|---|---|
| CYNO | PCGESEFLDTWNRETRCHQHKYCDPNLGLRVQQKGTSETDTIC |

(SEQ ID NO: 50)

| | |
|---|---|
| RHESUS | PCSESEFLDTWNRETRCHQHKYCDPNLGLRVQQKGTSETDTIC |

(SEQ ID NO: 51)

| | |
|---|---|
| HUMAN | PCGESEFLDTWNRETHCHQHKYCDPNLGLRVQQKGTSETDTIC |

CRD3

(SEQ ID NO: 52)

| | |
|---|---|
| CYNO | TCEEGLHCTSESCESCVPHRSCLPGFGVKQIATGVSDTICE |

(SEQ ID NO: 53)

| | |
|---|---|
| RHESUS | TCEEGLHCMSESCESCVPHRSCLPGFGVKQIATGVSDTICE |

(SEQ ID NO: 54)

| | |
|---|---|
| HUMAN | TCEEGWHCTSEACESCVLHRSCSPGFGVKQIATGVSDTICE |

CRD4

(SEQ ID NO: 55)

| | |
|---|---|
| CYNO | PCPVGFFSNVSSAFEKCRPWTSCETKDLVVQQAGTNKTDVVCG |

(SEQ ID NO: 55)

| | |
|---|---|
| RHESUS | PCPVGFFSNVSSAFEKCRPWTSCETKDLVVQQAGTNKTDVVCG |

(SEQ ID NO: 56)

| | |
|---|---|
| HUMAN | PCPVGFFSNVSSAFEKCHPWTSCETKDLVVQQAGTNKTDVVCG |

Differences from Human AKH3-CD40 Crystal Contact Resides

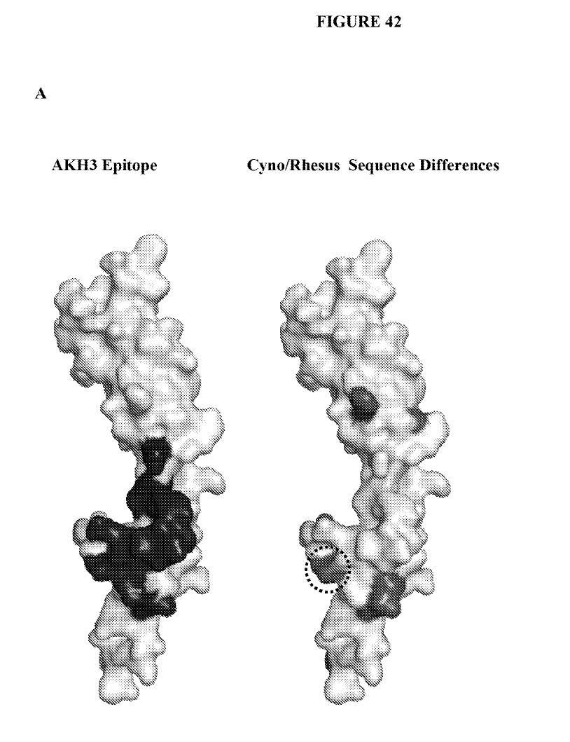

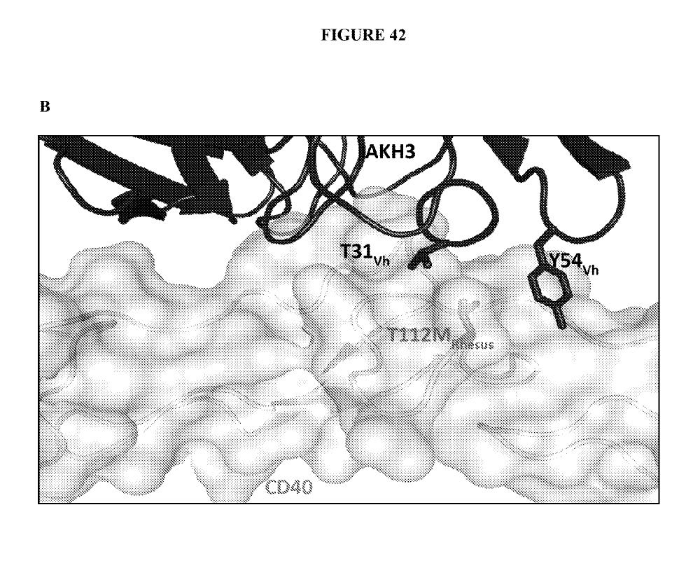

The comparable species cross-reactivity between human and cynomolgus monkey CD40, but only weak cross-reactivity to rhesus CD40 is explained by the mapping of the AKH3 epitope on the CD40 ECD. FIG. 42A shows the AKH3 epitope on the human CD40 ECD structure (underlined residues) side-by-side with a structural model highlighting the amino acid differences between human and nonhuman primate CD40 ECD (italicized residues). The designated residues show that the AKH3 epitope is highly conserved between human and cynomolgus monkey CD40, with a difference of only 6 amino acids and 5 are outside the AKH3 epitope. The same 6 residues distinguish human and rhesus CD40, however one additional key residue distinguishes rhesus from human and cynomolgus monkey CD40 and this residue clashes with the complementarity determining residues (CDR) of AKH3. (FIG. 42B).

### Example 34. Drug Target Polymorphisms

The reported single nucleotide polymorphisms (SNPs) in the human CD40 gene were compiled from the NCBI SNP database (www.ncbi.nlm.nih.gov/snp). A total of 405 SNPs were mapped onto the 18,479 base-pair reference CD40 gene sequence (NG_007279). Most of the SNPs were in the 5′ untranslated region, introns, and 3′ untranslated regions. Of the SNPs in the coding sequence, both synonymous and non-synonymous types were identified. The synonymous SNPs produce no change in amino acid sequence and these were omitted from subsequent consideration. The non-synonymous SNPs produced missense or frameshift changes. The collection of SNPs that affect the CD40 peptide sequence as well as the location of the change, and whether the sequences have already been cloned are included in Table 6. In addition, SNPs within introns that are predicted to affect splicing are also included in Table 6. The NCBI GeneView database (ncbi.nlm.nih.gov/projects/SNP/snp_ref.cgi?geneId=958) confirms most of the non-synonymous SNPs presented in Table 6.

TABLE 6
SNPs predicted to affect human CD40 protein sequence

| | | | | Plasmid Name |
|---|---|---|---|---|
| | | | Plasmid Name | (soluble |
| | | | (full length | extracellular |
| Amino acid | | | constructs for cell | domain |
| change | SNP identifier | Domain | surface expression) | constructs) |
| C8G | Rs113207193 | Signal peptide | | |
| A25S | Rs147677886 | Extracellular | CN914 | YL1143 |
| S65R | Rs202208745 | Extracellular | | YL1144 |
| D69E | Rs371950759 | Extracellular | | YL1145 |
| W71 shift-1 | Rs11478618 | Extracellular | | |
| C77F | Rs17855908 | Extracellular | CN915 | YL1146 |
| H78Q | Rs17177493 | Extracellular | CN916 | YL1147 |
| H80R | Rs376829285 | Extracellular | | YL1148 |
| C83R | Rs28931586 | Extracellular | | YL1149 |
| R90W | Rs144542285 | Extracellular | CN920 | YL1150 |
| S124L | Rs11569321 | Extracellular | pYL880 | YL1151 |
| I134V | Rs61760052 | Extracellular | pYL881 | YL1152 |
| I134L | | Extracellular | pYL882 | YL1153 |
| I134T | Rs371997367 | Extracellular | | YL1154 |
| V138F | Rs368921167 | Extracellular | | YL1155 |
| I142V | | Extracellular | | |
| F158L | Rs79661585 | Extracellular | pYL883 | YL1156 |
| S166R | Rs144600981 | Extracellular | pYL884 | YL1157 |
| I204V | Rs143037975 | Transmembrane | | |
| I208V | Rs199581355 | Transmembrane | | |
| Exon 8 skip/ | Rs371691887 | Intracellular | | |
| shift + 1 | | | | |
| A219T | Rs368619894 | Intracellular | | |
| K220R | Rs371799172 | Intracellular | | |
| P227A | Rs11086998 | Intracellular | | |
| Q252term | Rs199980487 | Intracellular | | |
| R270H | Rs139300926 | Intracellular | | |

The CD40 numbering begins with the initial methionine. The domain determination is based on a combination of software for identifying signal peptides and transmembrane domains.

Nine CD40 DNA sequences containing SNPs were cloned and expressed on 293E cells to evaluate binding of Exemplary Anti-CD40 Antibody 1. The prevalence of these SNPs is quite rare, believed to be at or below the 3% range. Exemplary Anti-CD40 Antibody 1 bound to 6 of the CD40 proteins encoded by DNA sequences containing SNPs comparably to WT CD40, but more weakly to H78Q and R90W (reduced plateau) and not quantifiably to C77F (data not shown). Since it was not known if the diminished binding was due to poor or absent surface expression on the 293E cells, subsequent experiments utilized 15 CD40 ECD SNPs expressed as soluble Fc fusion proteins and binding of Exemplary Anti-CD40 Antibody 1 and the Reference Anti-CD40 Antibody was evaluated by Octet Exemplary Anti-CD40 Antibody 1 bound to 13 of the CD40 proteins encoded by DNA sequences containing SNPs comparably to WT CD40, but more weakly to C77F (reduced plateau) and not quantifiably to C83R In contrast, the Reference Anti-CD40 Antibody did not bind quantifiably to C77F, H78Q or C83R indicating epitope differences from Exemplary Anti-CD40 Antibody 1 (Table 7).

TABLE 7
Binding to Soluble CD40 SNPs by Octet

| | | Reference anti- | Exemplary anti- |
|---|---|---|---|
| Amino acid change | EC Domain | CD40 Antibody | CD40 Antibody 1 |
| A25S | CRD1 | =WT | =WT |
| S65R | CRD2 | =WT | =WT |
| D69E | CRD2 | =WT | =WT |
| C77F | CRD2 | No binding | 50% reduced |
| | | | plateau |
| H78Q | CRD2 | No binding | =WT |
| H80R | CRD2 | =WT | =WT |
| C83R | CRD2 | No binding | No binding |
| R90W | CRD2 | =WT | =WT |
| S124L | CRD3 | =WT | =WT |
| I134V | CRD3 | =WT | =WT |
| I134L | CRD3 | =WT | =WT |
| I134T | CRD3 | =WT | =WT |
| V138F | CRD3 | =WT | =WT |
| F158L | CRD4 | =WT | =WT |
| S166R | CRD4 | =WT | =WT |

The location of these SNPs in the human CD40 ECD is shown below (CRD1: SEQ ID NO: 48; CRD2: SEQ ID NO: 51; CRD3: SEQ ID NO: 54; CRD4: SEQ ID NO: 56).

### CD40 Cysteine Rich Domains

CRD1:

(SEQ ID NO: 48)
ACREKQYLINSQCCSLCQPGQKLVSDCTEFTETECL

CRD2:

(SEQ ID NO: 51)
PCGESEFLDTWNRETHCH [symbol] H [symbol] C [symbol] GLRVQQKGTSETDTIC

CRD3:

(SEQ ID NO: 54)
TCEEGWHC [symbol] EAC [symbol] HRSCSPGFGVKQIATGVSDTICE

CRD4:

(SEQ ID NO: 56)
PCPVGFFSNVSSAFEKCHPWTSCETKDLVVQQAGTNKTDVVCG

### AKH3-CD40 Crystal Contact Residues (Italicized Above)

### Human CD40 SNPS (Underlined Above):

### A25S, S65R, D69E, C77F, H78G, H80R, C83R, R90W, S124L, I134V/L/T, V138F, I142V, F158L, S166R

### Example 35. Pharmacokinetics of Single Dose of Exemplary Anti-CD40 Antibody 1 in Cynomolgus Monkey

Exemplary Anti-CD40 Antibody 1 was formulated in 20 mM citrate, 150 mM NaCl (pH 6.0) and dosed intravenously to female cynomolgus monkeys at the dose levels of 1, 3 10, and 30 mg/kg, at dosing volume of 2.5 mL/kg. Blood samples were collected at multiple time points, 5 min and 12 hr post-dose on the same day and on Day 1 2, 4, 7, 10, 14, 21, 28, 35, 49 and 63 post-dose (n=5/time point, serial bleeds). Blood samples were kept undisturbed at room temperature for 30 min, processed to obtain serum, and stored at −70° C. until analysis using ELISA.

Serum concentrations versus time profiles at doses of 1, 3, 10, and 30 mg/kg were plotted. Exemplary Anti-CD40 Antibody 1 exhibited a bi-exponential elimination profile. The corresponding Exemplary Anti-CD40 Antibody 1 PK parameters are summarized in Tables 8, 9, 10, and 11.

TABLE 8
Exemplary Anti-CD40 Antibody 1 Serum PK Parameters Following
a Single IV Administration of 1 mg/kg to Cynomolgus Monkeys

| | AUCₗₐₛₜ | AUC_{inf} | *AUC_% | t_{1/2} | Cl | Vₛₛ |
|---|---|---|---|---|---|---|
| ID | (μg*day/mL) | (μg*day/mL) | (%) | (day) | (mL/day/kg) | (L/kg) |
| c2051 | 22.9 | 24.4 | 6.3 | 1.8 | 40.9 | 0.096 |
| c2052 | 29.3 | 29.8 | 1.8 | 1.9 | 33.5 | 0.091 |
| c2053 | 23.5 | 26.7 | 11.9 | 2.2 | 37.4 | 0.111 |
| c2054 | 33.5 | 37.1 | 9.7 | 3.1 | 26.9 | 0.11 |
| c2055 | 17.7 | 17.9 | 0.8 | 1.0 | 56.0 | 0.088 |
| Mean | 25.4 | 27.2 | 6.1 | 2.00 | 39.0 | 0.10 |
| STDEV | 6.1 | 7.1 | 4.8 | 0.76 | 10.8 | 0.01 |

*AUC percent of extrapolation

TABLE 9
Exemplary Anti-CD40 Antibody 1Serum PK Parameters Following
a Single IV Administration of 3 mg/kg to Cynomolgus Monkeys

| | AUCₗₐₛₜ | AUC_{inf} | *AUC_% | t_{1/2} | Cl | Vₛₛ |
|---|---|---|---|---|---|---|
| ID | (μg*day/mL) | (μg*day/mL) | (%) | (day) | (mL/day/kg) | (L/kg) |
| c3501 | 113.7 | 115.1 | 1.2 | 2.3 | 26.1 | 0.094 |
| c3502 | 88.0 | 89.7 | 1.8 | 2.0 | 33.5 | 0.104 |
| c3503 | 149.2 | 150.9 | 1.1 | 2.3 | 19.9 | 0.077 |
| c3504 | 128.2 | 132.2 | 3.1 | 2.7 | 22.7 | 0.09 |
| c3505 | 109.4 | 110.2 | 0.7 | 2.1 | 27.2 | 0.092 |
| Mean | 117.7 | 119.6 | 1.6 | 2.25 | 25.9 | 0.091 |
| STDEV | 22.7 | 23.2 | 0.9 | 0.27 | 5.1 | 0.01 |

*AUC percent of extrapolation

TABLE 10
Exemplary Anti-CD40 Antibody 1Serum PK Parameters Following
a Single IV Administration of 10 mg/kg to Cynomolgus Monkeys

| | AUCₗₐₛₜ | AUC_{inf} | *AUC_% | t_{1/2} | Cl | Vₛₛ |
|---|---|---|---|---|---|---|
| ID | (μg*day/mL) | (μg*day/mL) | (%) | (day) | (mL/day/kg) | (L/kg) |
| c4501 | 621.1 | 661.4 | 6.1 | 3.6 | 15.1 | 0.072 |
| c4502 | 1067.0 | 1090.3 | 2.1 | 6.5 | 9.2 | 0.082 |
| c4503 | 974.4 | 992.8 | 1.8 | 6.4 | 10.1 | 0.090 |
| c4504 | 1163.2 | 1219.8 | 4.6 | 7.8 | 8.2 | 0.086 |
| c4505 | 795.4 | 812.4 | 2.1 | 4.1 | 12.3 | 0.072 |
| Mean | 974.2 | 955.3 | 3.4 | 5.66 | 11.0 | 0.080 |
| STDEV | 217.1 | 221.6 | 1.9 | 1.79 | 2.8 | 0.01 |

*AUC percent of extrapolation

TABLE 11
Exemplary Anti-CD40 Antibody 1Serum PK Parameters Following
a Single IV Administration of 30 mg/kg to Cynomolgus Monkeys

| | AUCₗₐₛₜ | AUC_{inf} | *AUC_% | t_{1/2} | Cl | Vₛₛ |
|---|---|---|---|---|---|---|
| ID | (μg*day/mL) | (μg*day/mL) | (%) | (day) | (mL/day/kg) | (L/kg) |
| c5501 | 3522.8 | 3673.7 | 4.1 | 7.7 | 8.2 | 0.086 |
| c5502 | 3588.3 | 3605.4 | 0.5 | 7.4 | 8.3 | 0.096 |
| c5503 | 4262.6 | 4277.9 | 0.4 | 7.1 | 7.0 | 0.081 |
| c5504 | 4090.9 | 4098.1 | 0.2 | 6.2 | 7.3 | 0.076 |
| c5505 | 4817.3 | 4862.7 | 0.9 | 8.5 | 6.2 | 0.079 |
| Mean | 4056.4 | 4103.5 | 1.2 | 7.39 | 7.4 | 0.083 |
| STDEV | 530.7 | 509.8 | 1.6 | 0.84 | 0.9 | 0.01 |

*AUC percent of extrapolation

### Example 36. Dose Linearity in Cynomolgus Monkeys

A multiple dose PK study was conducted at the dose levels of 1, 3 10, and 30 mg/kg via IV administration to cynomolgus monkeys. The mean serum concentration versus time profiles of Exemplary Anti-CD40 Antibody 1 at the four dose levels were plotted. The corresponding Exemplary Anti-CD40 Antibody 1 PK parameters are summarized in Table 12 below.

TABLE 12
Exemplary Anti-CD40 Antibody 1 Mean Serum PK Parameters
Following a Single IV Administration of 1, 3, 10, 30 mg/kg to
Cynomolgus Monkeys

| | AUC_{inf} | | | | |
|---|---|---|---|---|---|
| Dose | (μg * day/ | AUC_{inf}/dose | t_{1/2} | Cl | Vₛₛ |
| (mg/kg) | mL) | (μg * day/mL/kg) | (day) | (mLday/kg) | (L/kg) |
| 1 | 27.2 | 27.2 | 2.0 | 39.1 | 0.100 |
| 3 | 119.6 | 39.9 | 7.3 | 25.9 | 0.091 |
| 10 | 955.3 | 95.5 | 5.7 | 11.0 | 0.080 |
| 30 | 4103.0 | 136.8 | 7.4 | 7.4 | 0.083 |

As Table 12 indicates, both clearance and half-life of Exemplary Anti-CD40 Antibody 1 are dose-dependent over the 1-30 mg/kg dose range. As dose increased, clearance decreased and half-life increased, consistent with a target mediated drug disposition (TMDD) profile. The clearance mechanism of Exemplary Anti-CD40 Antibody 1 consisted of both first order and target mediated pathways. Clearance ranged from 7.4 to 39 mL/day/kg, and half-life ranged from 2.2 to 7.8 days over the 1-30 mg/kg dose range. The volume of distribution was small (83-100 ml/kg), consistent across four dose levels, suggesting that Exemplary Anti-CD40 Antibody 1 is primarily restricted to the extracellular space.

### Example 37. Assessing Binding of Anti-CD40 Antibodies to B Cells in Whole Blood

Binding to cell surface CD40 on primary B cells was measured by immunofluorescent staining of human whole blood from four donors with various concentrations of fluorochrome A647-conjugated anti-CD40 antibodies and flow cytometry analysis. The staining cocktail included FITC-conjugated-anti-CD20 which was used to gate on the B lymphocytes, a key CD40-expressing cell type. EC50 values were derived from graphs of the A647 geometric mean fluorescence intensity versus mAb concentration.

TABLE 13
Alexa 647 conjugated mAbs binding to B cells in whole blood
(EC50 values in nM)

| Anti-CD40 Antibody | Donor 1 | Donor 2 | Donor 3 | Donor 4 |
|---|---|---|---|---|
| Exemplary Ab 1 | 0.39 | 0.30 | 0.28 | 0.32 |
| Reference Ab 1 (IgG4PE) | 1.12 | 0.85 | 0.51 | 0.59 |
| Reference Ab 2 (IgG1 ala ala)* | ~10.15 | ~8.81 | ~6.57 | ~10.15 |
| Reference Ab 3 (IgG4) | 1.01 | 0.74 | 0.58 | 0.52 |

*EC50 was estimated from incomplete curves where saturation was not achieved.

Exemplary Anti-CD40 Antibody 1 exhibits the best binding, with the lowest EC50 values of the four antibodies tested in this experiment. Reference anti-CD40 Ab 2 binds much more weakly and saturation of CD40 on B cells was not achieved even at the highest concentration used (5 μg/mL).

### Example 38. Inhibition of Soluble CD40L-Stimulated B Cell Activation in Whole Blood

Evaluation of CD40L-dependent B cell activation in whole blood was performed using recombinant soluble CD40 ligand (rsCD40L) to stimulate CD40 signaling. T helper cell activation of B cells by CD40 signaling is enhanced by co-engagement of antigen (B cell receptor signaling) or T cell-derived cytokines, notably IL-4. Thus the functional potency of anti-CD40 antibodies in blocking CD40L-induced B cell activation was evaluated in assays of human whole blood stimulated with rsCD40L and IL-4, with B cell activation measured by FACS analysis. A concentration of rsCD40L was used that stimulated nearly maximal induction of the B cell activation markers CD69 and CD54 with similar results obtained for each marker.

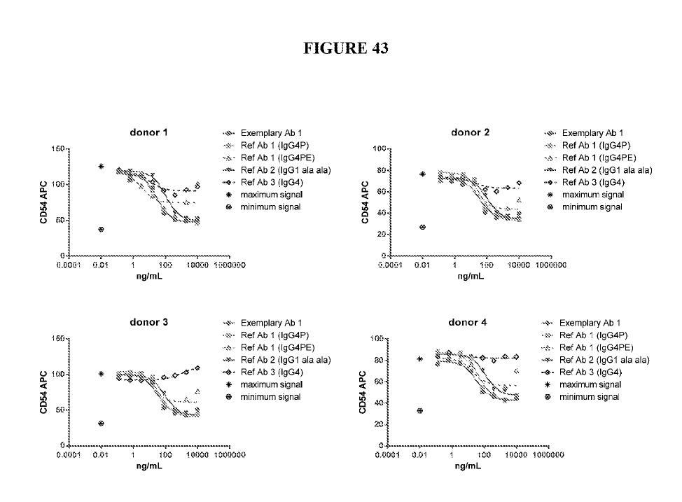

FIG. 43 shows representative inhibition curves for rsCD40L-induced expression of the activation marker CD54 on B cells in whole blood from four normal healthy donors. This data is also presented in terms of the IC50 values in Table 14. The staining cocktail included FITC-conjugated-anti-CD20 which was used to gate on the B lymphocytes, a key CD40-expressing cell type and APC-conjugated CD54, for detection of this rsCD40L-induced activation marker on the B cells. IC₅₀ values were derived from graphs of the CD54 APC geometric mean fluorescence intensity versus mAb concentration.

TABLE 14
Potency in whole blood (IC50 values in ng/mL)

| Anti-CD40 Antibody | Donor 1 | Donor 2 | Donor 3 | Donor 4 |
|---|---|---|---|---|
| Exemplary Ab 1 | 28.43 | 32.87 | 31.40 | 33.39 |
| Reference Ab 1 (IgG4P) | 28.54 | 44.47 | 43.09 | 48.92 |
| Reference Ab 1 (IgG4PE) | ICI | ICI | ICI | ICI |
| Reference Ab 2 (IgG1 ala ala) | 106.20 | 108.90 | 95.08 | 118.60 |
| Reference Ab 3 (IgG4) | ICI | ICI | ICI | ICI |

ICI = incomplete inhibition;
Curve fit R squared value <0.950

Exemplary Anti-CD40 Antibody 1, Reference anti-CD40 Antibody 1 (IgG4P), and Reference anti-CD40 Antibody 2 (IgG1 ala ala) fully inhibited B cell activation. The functional potency of Exemplary anti-CD40 Antibody 1 was comparable with that of Reference anti-CD40 Antibody 1 (IgG4P), whereas the Reference anti-CD40 Antibody 2 (IgG1 ala ala) was less potent. In addition, Reference anti-CD40 Antibody 1 (IgG4PE) and Reference anti-CD40 Antibody 3 (IgG4) exhibited incomplete inhibition due to anti-CD40 antibody induced activation at higher concentrations that negates any inhibitory activity.

### Example 39. Assessment of Agonistic Activity in Whole Blood

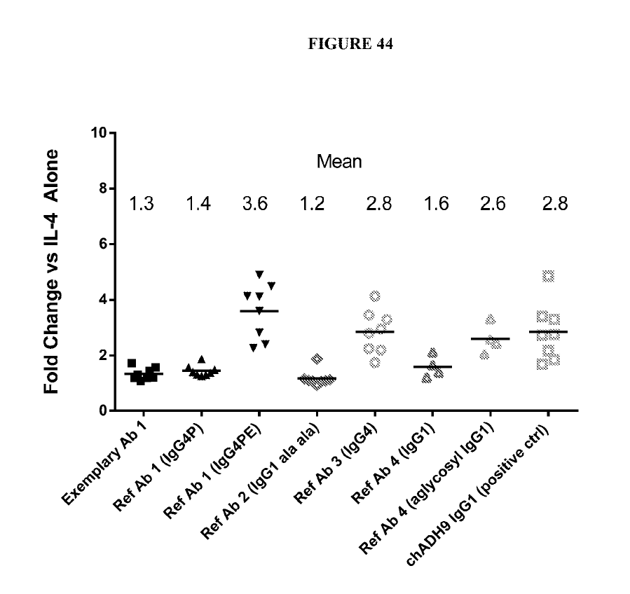

Agonistic activity of the anti-CD40 antibodies was evaluated after overnight culturing of whole blood in the presence of IL-4 and various concentrations of soluble anti-CD40 mAbs as measured by immunofluorescent staining for the induction of B cell activation markers, CD69 and CD95. A fluorochrome conjugated-anti-CD19 antibody was included in the staining cocktail to enable gating on the B cell population.

Whereas an Exemplary anti-CD40 Antibody 1 was only minimally agonistic for B cell activation in human whole blood cultures, several other anti-CD40 antibodies (Ref Ab 1 IgG4PE, Ref Ab 3, and Ref Ab 4) had agonistic profiles similar to the chADH9 positive control (FIG. 44).

### Example 40. Further Experiments to Assess Effect of Removal of N-Linked Glycosylation Site

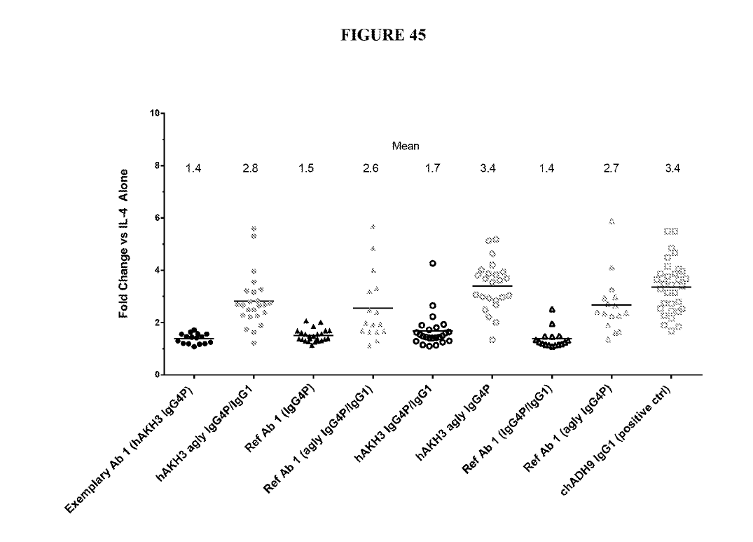

This example furthers the studies described in Example 23 above. Additional matched sets of antibody constructs were produced to evaluate the agonistic potential of hAKH3 and Reference anti-CD40 Antibody 1 on aglycosyl IgG4P versus glycosylated IgG4P/IgG1 scaffolds in an attempt to dissect if the agonistic activity observed with the aglycosyl IgG4P/IgG1 constructs was caused by removal of the N-linked glycans or the addition of the IgG1 CH3 domain. A fully Fc-competent form of ADH9 (chADH9 IgG1) was included as a positive control. The presence of the IgG1 CH3 domain does not alter the agonistic profile whereas aglycosyl forms of hAKH3 and Reference anti-CD40 Antibody 1 are more agonistic than their glycosylated counterparts (FIG. 45).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antibody or antigen-binding fragment thereof that selectively binds to human CD40 and (i) binds to the same epitope on human CD40 as an antibody that has a heavy chain comprising amino acids 21-463 of SEQ ID NO:46 and a light chain comprising amino acids 23-236 of SEQ ID NO:38, and (ii) inhibits the interaction between human CD40 and human CD40 ligand.

2. An isolated antibody or antigen-binding fragment thereof that selectively binds to human CD40 at an epitope within cysteine-rich domain 2 (CRD2) and cysteine-rich domain 3 (CRD3); inhibits the humoral response to tetanus toxoid immunization in a primate without B cell depletion compared to vehicle; and/or does not elevate IL-12 serum levels in a primate compared to vehicle; and/or binds to a CD40 protein encoded by a DNA molecule containing the CD40 SNP C77F about 50% as well as to wild type human CD40 (SEQ ID NO:58); and/or binds to a CD40 protein encoded by a DNA molecule containing the CD40 SNP H78Q comparably as to wild type human CD40 (SEQ ID NO:58) and optionally has one or more of the following functions/activities:
(i) inhibits the interaction between human CD40 and human CD40 ligand;
(ii) has a K_{D}≦3 nM for cysteine-rich domains 2-3 of the extracellular domain of human CD40;
(iii) has an EC₅₀ value between 20 and 200 ng/mL for binding to B cells in human whole blood;
(iv) inhibits primary B cell activation by CD40L on Jurkat cells with an IC₅₀ of between 5 and 100 ng/mL;
(v) inhibits primary B cell activation in whole blood by soluble CD40L with an IC₅₀ of between 10 and 200 ng/mL;
(vi) does not agonize platelets stimulated by soluble CD40L compared with the anti-CD40 antibody, G28.5 antibody;
(vii) has less agonistic activity in a RAMOS B cell line compared to the anti-CD40 antibody, ADH9;
(viii) has less agonistic activity in whole blood cultures compared to the anti-CD40 antibody, ADH9;
(ix) has reduced binding as compared to a wild type IgG1 to CD16a of about 200 fold, to CD32a and CD32b of about 5 fold, and CD64 of about 150 fold; and/or
(x) binds to a CD40 protein encoded by a DNA sequence that contains at least one of the following human CD40 SNPs: A25S; S124L; I134V; F158L; S166R; S65R; D69E; H78Q; H80R; R90W; I134L; I134T; and V138F with an EC₅₀ of 100-650 ng/mL.

3. The isolated antibody or antigen-binding fragment thereof of claim 1, wherein the antibody or antigen-binding fragment thereof binds to cynomolgus CD40 but binds to rhesus CD40, murine CD40, and rat CD40 with a lower binding affinity than to human or cynomolgus CD40.

4. The isolated antibody or antigen-binding fragment thereof of claim 2, wherein the antibody or antigen-binding fragment thereof:
(i) binds to human CD40 at an epitope within amino acids 70 to 130 of SEQ ID NO:58;
(ii) inhibits the interaction between human CD40 and human CD40 ligand;
(iii) has a KD of 0.1 nM to 3 nM for CRDs 2-3 of the extracellular domain of human CD40;
(iv) binds to a CD40 protein encoded by a DNA molecule containing the CD40 SNP C77F about 50% as well as to wild type human CD40 (SEQ ID NO:58); and
(v) binds to a CD40 protein encoded by a DNA molecule containing the CD40 SNP H78Q comparably as to wild type human CD40 (SEQ ID NO:58).

5. An isolated antibody or antigen-binding fragment thereof that (i) selectively binds to human CD40, and (ii) comprises a variable heavy (VH) domain comprising a heavy chain complementarity determining region 1 (CDR1), a heavy chain CDR2, and a heavy chain CDR3, wherein:
the heavy chain CDR1 consists of the amino acid sequence TFPIE (SEQ ID NO: 61) or the amino acid sequence set forth in SEQ ID NO: 61 with a substitution at one or two amino acid positions;
the heavy chain CDR2 consists of the amino acid sequence NFHPYNDDTKYNEKFKG (SEQ ID NO:62) or the amino acid sequence set forth in SEQ ID NO:62 with a substitution at one, two, three, or four amino acid positions; and
the heavy chain CDR3 consists of the amino acid sequence RGKLPFDS (SEQ ID NO:63) or the amino acid sequence set forth in SEQ ID NO:63 with a substitution at one, two, or three amino acid positions.

6-7. (canceled)

8. The isolated antibody or antigen-binding fragment thereof of claim 5, wherein the antibody or antigen-binding fragment thereof comprises a variable light (VL) domain comprising a light chain CDR1, a light chain CDR2, and a light chain CDR3, wherein:
the light chain CDR1 consists of the amino acid sequence RASQDISNYLN (SEQ ID NO:64) or the amino acid sequence set forth in SEQ ID NO:64 with a substitution at one, two, three, or four amino acid positions;
the light chain CDR2 consists of the amino acid sequence FTSRLRS (SEQ ID NO:65) or the amino acid sequence set forth in SEQ ID NO:65 with a substitution at one or two amino acid positions; and
the light chain CDR3 consists of the amino acid sequence QQDRKLPWT (SEQ ID NO:66) or the amino acid sequence set forth in SEQ ID NO:66 with a substitution at one, two, or three amino acid positions.

9-10. (canceled)

11. The isolated antibody or antigen-binding fragment thereof of claim 8, wherein:
the heavy chain CDR1 consists of the amino acid sequence TFPIE (SEQ ID NO: 61);
the heavy chain CDR2 consists of the amino acid sequence NFHPYNDDTKYNEKFKG (SEQ ID NO:62);
the heavy chain CDR3 consists of the amino acid sequence RGKLPFDS (SEQ ID NO:63);
the light chain CDR1 consists of the amino acid sequence RASQDISNYLN (SEQ ID NO:64);
the light chain CDR2 consists of the amino acid sequence FTSRLRS (SEQ ID NO:65); and
the light chain CDR3 consists of the amino acid sequence QQDRKLPWT (SEQ ID NO:66).

12. An isolated antibody or antigen-binding fragment thereof that (i) selectively binds to human CD40, and (ii) comprises a variable heavy (VH) domain that is at least 80% identical to the amino acid sequence of SEQ ID NO:33.

13-14. (canceled)

15. The isolated antibody or antigen-binding fragment thereof of claim 12, wherein the VH domain is identical to the amino acid sequence of SEQ ID NO:33.

16. The antibody or antigen-binding fragment thereof of claim 12, wherein the antibody comprises a heavy chain comprising amino acids 21-463 of SEQ ID NO:46.

17. The isolated antibody or antigen-binding fragment thereof of claim 12, wherein the antibody or antigen-binding fragment thereof comprises a variable light (VL) domain that is at least 80% identical to the amino acid sequence of SEQ ID NO:34.

18-19. (canceled)

20. The isolated antibody or antigen-binding fragment thereof of claim 17, wherein the VH domain is identical to the amino acid sequence of SEQ ID NO:33 and the VL domain is identical to the amino acid sequence of SEQ ID NO:34.

21. The antibody or antigen-binding fragment thereof of claim 17, wherein the heavy chain comprises amino acids 21-463 of SEQ ID NO:46 and the light chain comprises amino acids 23-236 of SEQ ID NO:38.

22. The antibody or antigen-binding fragment thereof of claim 1, wherein the antibody is a humanized antibody.

23. (canceled)

24. The antibody or antigen-binding fragment thereof of any of claim 1, wherein the antibody is a single chain antibody.

25. The antibody or antigen-binding fragment thereof of claim 1, wherein the antibody is a polyclonal antibody, a chimeric antibody, an F_{ab} fragment, an F_{(ab′)2} fragment, an F_{ab′} fragment, an F_{sc} fragment, an Fᵥ fragment, an scFv, an sc(Fv)2, or a diabody.

26-27. (canceled)

28. A nucleic acid encoding the antibody or antigen-binding fragment thereof of claim 1.

29. An isolated cell that produces the antibody or antigen-binding fragment thereof of claim 1.

30. A pharmaceutical composition comprising the antibody or antigen-binding fragment thereof of claim 1 and a pharmaceutically acceptable carrier.

31-33. (canceled)

34. A method of inhibiting hyperactivation of B or T cells in a human subject in need thereof, the method comprising administering to the subject in need thereof an effective amount of the antibody or antigen-binding fragment thereof of claim 1.

35. A method of treating or preventing an autoimmune disease in a human subject in need thereof, comprising administering to the subject in need thereof an effective amount of the antibody or antigen-binding fragment thereof of claim 1.

36. The method of claim 35, wherein the autoimmune disease is selected from the group consisting of Sjogren's syndrome, systemic lupus erythematosus, lupus nephritis, discoid lupus, acquired hemophilia, systemic sclerosis (scleroderma), Crohn's disease, ulcerative colitis, Graves disease, immune thrombocytopenic purpura, rheumatoid arthritis, asthma, vasculitis, pemphigoid, atopic dermatitis, and hemolytic anemia.

37-40. (canceled)

41. A method of treating or preventing transplant rejection in a human subject in need thereof, comprising administering to the subject in need thereof an effective amount of the antibody or antigen-binding fragment thereof of claim 1.

42. (canceled)

43. A method of treating or preventing graft versus host disease in a human subject in need thereof, comprising administering to the subject in need thereof an effective amount of the antibody or antigen-binding fragment thereof of claim 1.

44. A method of treating or preventing Alzheimer's disease in a human subject in need thereof, comprising administering to the subject in need thereof an effective amount of the antibody or antigen-binding fragment thereof of claim 1.

45. A method of treating or preventing neuromyelitis optica in a human subject in need thereof, comprising administering to the subject in need thereof an effective amount of the antibody or antigen-binding fragment thereof of claim 1.

46. A method of treating or preventing myasthenia gravis in a human subject in need thereof, comprising administering to the subject in need thereof an effective amount of the antibody or antigen-binding fragment thereof of claim 1.

47. A method of treating or preventing amyotrophic lateral sclerosis in a human subject in need thereof, comprising administering to the subject in need thereof an effective amount of the antibody or antigen-binding fragment thereof of claim 1.

48. A method of treating or preventing hemophilia with inhibitors in a human subject in need thereof, comprising administering to the subject in need thereof an effective amount of the antibody or antigen-binding fragment thereof of claim 1.
